(19) United States
(12) Patent Application Publication
(10) Pub. No.: US 2023/0312639 A1
(43) Pub. Date: Oct. 5, 2023

(54) GPER PROTEOLYTIC TARGETING CHIMERAS

(71) Applicant: University of Iowa Research Foundation, Iowa City, IA (US)

(72) Inventors: Aliasger K. Salem, Coralville, IA (US); Edward J. Filardo, Coralville, IA (US); Anh S. Lu, Middleton, WI (US); Milad Rouhimoghadam, Iowa City, IA (US)

(21) Appl. No.: 17/920,645

(22) PCT Filed: Apr. 23, 2021
(86) PCT No.: PCT/US2021/028900

§ 371 Date: Oct. 21, 2022

Related U.S. Application Data
(60) Provisional application No. 63/014,410, filed on Apr. 23, 2020.
(60) Provisional application No. 63/144,783, filed on Feb. 2, 2021.

(51) Int. Cl. C07J 43/00; A61K 47/60; A61P 35/00

(52) U.S. Cl. CPC C07J 43/00; A61K 47/60; A61P 35/00

(57) ABSTRACT

A molecule comprising a G-protein coupled estrogen receptor (GPER) ligand coupled to a linker coupled to an E3 ubiquitin ligase ligand and methods of using the molecule are provided. In one embodiment, the GPER ligand is estradiol and the E3 ubiquitin ligase ligand is (S,R,S)- AHPC.

### CROSS-REFERENCE TO RELATED APPLICATIONS

This application claims the benefit of the filing date of U.S. application No. 63/014,410, filed on Apr. 23, 2020 and U.S. application No. 63/144,783, filed on Feb. 2, 2021, the disclosures of which are incorporated by reference herein.

### BACKGROUND

Epidemiological, clinical and preclinical evidence suggests that breast cancer is an estrogen-driven malignancy (Germain et al., 2011; Parl et al., 2018; Rothenberger et al., 2018; Rugo et al., 2016; Yue et al., 2016). This explains the broad success of anti-estrogens as an effective adjuvant treatment for early stage ER (+) breast cancer (DePlacido et al., 2018; Tremont et al., 2017). Two classes of pharmacological agents are used to antagonize estrogen action: 1) compounds that inhibit aromatase (aromatase inhibitors, Als), the enzyme that introconverts androgens to estrogens and 2) estrogen mimetics (ER antagonists) that competitively block the interaction of estrogen with the ER. In order to be effective, Als or ER antagonists must be administered over a long-term drug schedule that lasts 3-5 years or longer. Ultimately, prolonged use of anti-estrogen therapy is associated with undesirable and sometimes intolerable side effects, including menopausal symptoms, osteoporosis, ostealgia and arthralgia (Yang et al., 2013) In addition, long-term use of ER antagonists is linked with an increased risk of endometrial cancer (Hu et al., 2015; Jones et al., 2012) and thrombosis (Cosman et al., 2005).

De novo or acquired drug resistance further limits the use of anti-estrogen therapy, with resistance occurring in more than 20% of patients that are treated (Augusto et al., 2018; Clarke et al., 2001; Haque et al., 2019; Lei et al., 2019). While de novo resistance is attributed to intratumoral heterogeneity in ER expression at the time of diagnosis (Reinhardt et al., 2017), acquired resistance reflects tumor heterogeneity that evolves due to the selective pressure applied by anti-estrogens during treatment. Examples include: a) the selection of mutations which result in a loss of drug-receptor interaction (Fan et al., 2015) or drive estrogen-independent ER-mediated gene transactivation (Barone et al., 2010; GRCIA-Becerra et al., 2012), b) epigenetic silencing of the ER promoter (Achinger-Kaecke et al., 2020) and c) transcriptional upregulation of compensatory genes that drive estrogen-independent growth by regulating cell cycle activity (Thngavel et al., 2011) or signaling activity downstream of epidermal growth factor receptors (EGFRs) (Guilano et al., 2011). Yet another mechanism of anti-estrogen resistance is provided by the G-protein coupled estrogen receptor (GPER). This more recently appreciated estrogen receptor has clear importance for breast cancer biology and treatment (Rouhimoghadm et al., 2020). Unlike nuclear estrogen receptors that primarily reside intracellularly and function as ligand-induced transcription factors, GPER is a Gs-coupled heptahelical transmembrane receptor that is located in the plasma membrane and intracellular membranes and promotes rapid pre-genomic actions, including activation of adenylyl cyclase (Filardo et al., 20002) and transactivation of EGFRs (Filardo et al., 2000). Stimulation of GPER contributes to the activation of signaling effectors downstream of EGFRs such as Ras, PI3K, AKT, and Erk½ and are involved in cell proliferation, survival, invasion, and resistance to endocrine therapy (Peperman et al., 2019) Analysis of GPER expression in primary breast tumors demonstrates that its presence is linked with disease progression (Filardo et al., 2006), survival of breast cancer stem cells (Chan et I., 2020), and tamoxifen resistance (Ignatov et al., 2011; Rouhimoghadam et al., 2018; Yin et al., 2017). Furthermore, its expression is commonly retained in triple-negative breast cancers which lack ER, PR, and her-2/neu (Steiman et al., 2013) Thus, GPER broadens an ER-centric view of estrogen responsiveness (Filardo et al., 2018) and disrupts the binary rubric that guides the rational assignment of adjuvant therapy for breast cancer. This is particularly important for patients receiving endocrine therapy because “partial” (tamoxifen) and “pure” (fulvestrant and raloxifene) ER antagonists function as GPER agonists (Filardo et al., 2000; Petrie et al., 2013).

Current clinical guidelines suggest the use of fulvestrant as a second-line therapy for endocrine-resistant breast cancer (Nathan et al., 2017; Sammons et al., 2019). Fulvestrant is an estrogen mimetic that functions as a competitive inhibitor for ER but also acts as a selective ER degrader (SERD) (Pike et al., 2001). Fulvestrant is the only FDA-approved drug in this class and upon binding ER, destabilizes its interaction with associated heat shock chaperone proteins resulting in the degradation of ER at the 26S proteasome (Callige et al., 2006). In this manner, fulvestrant acts to reduce the bioavailability of its drug target, thus reducing the risk of secondary drug resistance. However, a major limitation of fulvestrant is its poor bioavailability following oral delivery; necessitating painful monthly intramuscular injections (Robertson et al., 2007).

A method for selective degradation of the ER has been achieved by the use of PROteolytic TArgeting Chimeras (PROTACs) that utilize the ubiquitin-proteasome pathway (Cyrus et al., 2011; Huang et al., 2016). In general, PROTACs are hetero-bifunctional compounds consisting of two functional binding domains that act to link a protein of interest directly to the ubiquitination machinery. They are comprised of a targeting domain that is coupled via a chemical spacer to an E3 ubiquitin ligase recognition motif. Upon binding, the PROTAC polyubiquitinylates its target and directs it towards the 26S proteasome for degradation. PROTACs offer the advantage that they have been used to target a broad spectrum of cytoplasmic, nuclear, and plasma membrane proteins (Sun et al., 2019), albeit efficient PROTAC degradation, requires a systematic approach to optimize the spatial positioning of the targeting and E3 ligase recruitment domains (Bondeson et al., 2018). PROTACs provide the added benefit that they offer high specificity and processivity for their intended target protein; thus, effectively reducing the concentration of target molecules and decreasing the likelihood that drug-resistant mutants may evolve. This strategy has been used successfully to down-modulate ER and ER-PROTACs have been developed with targeting domains consisting of various estrogen derivatives including 17β-estradiol (17b-E2) (Rodriguez-Gonzales et al., 2008), endoxifen (Dragovich et al., 2020), raloxifene (Hu et al., 2019), and tamoxifen (Fan, 2020); each connected with different chemical spacers to protein or small molecule E3 ligase recruitment motifs. While ERα, ERβ and GPER differ in their binding affinities for natural and synthetic estrogens (Prissnitz et al., 2015), they each show a high binding affinity for 17β-estradiol (E2) with dissociation constants measured in the low nanomolar range.

### SUMMARY

The disclosure provides for PROTACs capable of inactivating G-protein coupled estrogen receptor (GPER). In one embodiment, the disclosure provides for an orally-delivered, small-molecule PROTAC capable of inactivating, or not activating, GPER, e.g., useful to treat cancers, such as triple negative breast cancer (TNBC), one of the most difficult-to-treat breast cancers. TNBC is a type of breast cancer which has no effective therapeutic targets and in which GPER is expressed commonly (>80% of TNBC). The GPER-PROTACs disclosed herein enhance the proteolytic degradation of GPER, e.g., in TNBC and so delay or inhibit cancer progression, thus evidencing that endocrine therapy can be employed for TNBC. GPER-PROTACs may also be useful in other cancers, such as other types of breast cancers or gynecological cancers, e.g., in patients currently judged to be ineligible for endocrine therapy. GPER-PROTACs may also be employed in conjunction with existing therapies (e.g., aromatase inhibitors). In one embodiment, the GPER-PROTACs are orally-delivered. In one embodiment, the GPER-PROTAC is an E2 based PROTAC, e.g., UI-EP001 or UI-EP002), that interacts with and degrades ERα, ERβ and GPER, e.g., it is a pan-estrogen receptor inhibitor. In one embodiment, dual-specificity PROTACs interact with the plasma membrane and intracellular estrogen receptors and promote ubiquitin-proteasome dependent degradation.

PROTACs for inactivating GPER are evaluated using, for instance, a competitive radioreceptor binding assay with [3H]-17β-estradiol (17β-E2) as tracer, to determine GPER specific binding in plasma membranes from target cells, e.g., breast cancer cells. PROTACs for inactivating GPER are also evaluated for their ability to promote proteasomal degradation of GPER in cancer cell lines, for example, breast cancer cell lines, and their anti-oncogenic efficacy in animal models such as a spontaneous cancer model using small primary TNBC tumors. As disclosed herein, an exemplary PROTAC for inactivating GPER, UIEP001, was shown to decrease native and recombinant GPER plasma membrane protein, and to reduce steady state expression of HA-GPER but not HA-β1AR.

In one embodiment, the disclosure provides a G-protein coupled estrogen receptor (GPER) ligand coupled to a ligand for an E3 ligase. In one embodiment, the disclosure provides a G-protein coupled estrogen receptor (GPER) ligand coupled, e.g., chemically linked via a covalent bond, to a linker which in turn is coupled, e.g., chemically linked via a covalent bond, to a ligand for an E3 ligase. In one embodiment, the GPER ligand comprises 17β-estradiol, estrone, a phytoestrogen, a xenoestrogen, estriol, estriol 3-sulfate, estriol 17-sulfate, G-1, G-15, G-36, genistein or quercetin. In one embodiment, the phytoestrogen comprises a flavone, isoflavone, lignin saponin, coumestin, or stilbene. In one embodiment, the GPER ligand comprises a bisphenol, alkylphenol, methoxyphenol, polychlorinated biphenyl, or dioxin. In one embodiment, the GPER ligand is a GPER antagonist. In one embodiment, the GPER ligand is a GPER agonist. In one embodiment, the GPER ligand is not 2-cyclohexyl-4-isopropyl-N-(4-methoxybenzyl)aniline. In one embodiment, the GPER PROTAC does not have formula (II) or (III)). In one embodiment the GPER ligand comprises 2-cyclohexyl-4-isopropyl-N-(4-methoxybenzyl)aniline. In one embodiment, the GPER ligand does not bind ER. In one embodiment, the E3 ligase ligand is a Von Hippel ligase (VHL) ligand. In one embodiment, the E3 ligase ligand comprises lenalidomide, pomalidomide, iberdomide, (S,R,S)-AHPC, thalidomide, VH-298, CC-885, E3ligase ligand 8, TD-106, VL285, VH032, VH101, VH298, VHL ligand 4, VHL ligand 7 (see Scheepstra et al., Comput. Struct. Biotech. J., 17:160 (2019), the disclosure of which is incorporated by reference herein)), VHL-2 ligand 3, E3 ligase ligand 3, E3 ligase ligand 2, cereblon, CC-122, or BC-1215. In one embodiment, the linker has a chain having 2 to 200 atoms. In one embodiment, the linker has a chain having 5 to 25 atoms. In one embodiment, the linker has a chain having 25 to 50 atoms. In one embodiment, the linker has a chain having 30 to 50 atoms. In one embodiment, the linker has a chain having 50 to 100 atoms. In one embodiment, the linker is an alkyl linker. In one embodiment, the linker is a heteroalkyl linker. In one embodiment, the linker comprises polyethylene glycol (PEG), e.g., 2, 3, 4, or 5 PEG units, and optionally one or more other groups such as an amido group. In one embodiment, the linker comprises 6,7,8,9,10, 11, 12, 13, 14 or 15 PEG units, and optionally one or more other groups such as an amido group. In one embodiment, the linker comprises a cleavable PEG linker, e.g., one having a disulfide linkage. In one embodiment, the linker is of a length that can span the membrane of a vertebrate cell, e.g., it is at least 5 to 10 nm in length. In one embodiment, the linker comprises at least 3 PEG units, e.g., (OCC)₃. In one embodiment, the linker comprises at least 4 PEG units, e.g., (OCC)₄. In one embodiment, the linker has at least 5 PEG units. In one embodiment, the linker comprises C₁-C₁₀alkyl(PEG)n. at least 4 PEG units, e.g., (OCC)_{4,} In one embodiment, the linker comprises (PEG)ₙNH(CO)(PEG)ₘ, wherein n and m are independently 1 to 15, In one embodiment, n and m are independently 3 to 10. In one embodiment, n is 5 to 10 and m is 3 to 6.

In one embodiment, the linker has a backbone having a chain length of 5 to 200 atoms as counted in a linear path between the GPER ligand and the E3 ubiquitin ligase ligand. In one embodiment, the linker has a backbone having a chain length of 15 to 50 atoms as counted in a linear path between the GPER ligand and the E3 ubiquitin ligase ligand. In one embodiment, the linker has a backbone having a chain length of 20 to 50 atoms as counted in a linear path between the GPER ligand and the E3 ubiquitin ligase ligand. In one embodiment, the linker has a backbone having a calculated length of 8 to 300 angstroms as determined from summing bond lengths in a linear path between the GPER ligand and the E3 ubiquitin ligase ligand.

In one embodiment, the linker has a backbone having a calculated length of 25 to 75 angstroms as determined from summing bond lengths in a linear path between the GPER ligand and the E3 ubiquitin ligase ligand. In one embodiment, the linker has the structure:

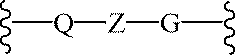

wherein Q is a bond or a divalent group that forms a covalent linkage to the GPER ligand; Z is a linear chain comprising one or more alkyl, aryl, heteroalkyl, heteroaryl, alkyloxy, alkylamino, alkylglycol, carbonyl, thiocarbonyl, acyl, carbamate, urea, thiocarbamate, thiourea, dithiocarbamate, aminocarbonyl, amide, ester, thioester, thioamide, amine, oxygen, sulfur, sulfone, or sulfoxide, in divalent form; G is a bond or a divalent group that forms a covalent linkage to the E3 ubiquitin ligase ligand. In one embodiment, the linker has the structure:

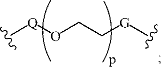

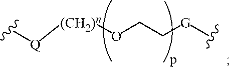

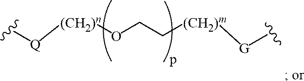

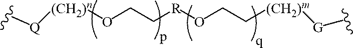

Wherein Q is a bond or a divalent group that forms a bond to the GPER ligand;
R is an alkyl, aryl, heteroalkyl, heteroalkyl, heteroaryl, alkyloxy, alkylamino, alkylglycol, carbonyl, thiocarbonyl, acyl, carbamate, urea, thiocarbamate, thiourea, dithiocarbamate, aminocarbonyl, amide, ester, thioester, thioamide, amine, oxygen, sulfur, sulfone, or sulfoxide, in divalent form; G is a bond or a divalent group that forms a bond to the E3 ubiquitin ligase ligand; and m, n, p, and q, if present, are each independently an integer from 0 to 50 provided that at least one of m, n, p, and q is an integer greater than 0. In one embodiment, Q and G are, independently, carbonyl, thiocarbonyl, acyl, carbamate, urea, thiocarbamate, thiourea, dithiocarbamate, aminocarbonyl, amide, ester, thioester, thioamide, sulfone, or sulfoxide, in divalent form. In one embodiment, Q and G are, independently, a carbonyl, or an acyl selected from the group consisting of acetyl, 2-hydroxyacetyl, 2-aminoacetyl, propionyl, 3-hydroxypropanoyl, 3-aminiopropanoyl, butanoyl, 4-hydroxybutanoyl, and 4-aminobutanoyl, each of which is in divalent form.

In one embodiment, m and n, if present, are each independently an integer from 0 to 2, and Q and G are each independently carbonyl, thiocarbonyl, acetyl, 2-hydroxyacetyl, 2-aminoacetyl, propionyl, 3-hydroxypropanoyl, 3-aminiopiopanoyl, butanoyl, 4-hydroxybutanoyl, 4-aminobutanoyl, carbamate, urea, thiocarbamate, thiourea, dithiocarbamate, aminocarbonyl, amide, ester, thioester, thioamide, sulfone, or sulfoxide, each of which is in divalent form. In one embodiment, Z is hydrophilic. In one embodiment, the linker has the structure

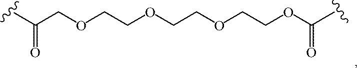

which provides a backbone having a length of 13 atoms as counted in a linear path. In one embodiment, the linker has the structure

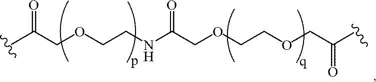

wherein p and q are each independently an integer from 0 to 50. In one embodiment, the linker comprises one or more of a divalent alkyl group, a divalent heteroalkyl group, or a divalent polyethylene glycol (PEG), or one or more of each.

In one embodiment, the linker comprises a 5 or more divalent ethoxy (—CH₂CH₂O—) groups. In one embodiment, the linker comprises a 1 or more heteroatoms for every 2 carbons and no alkyl chain longer than butyl. In one embodiment, the linker attaches to the GPER ligand via an oxygen, nitrogen, sulfur, carbonyl, or ethynyl of the GPER ligand, and the linker attaches to the E3 ubiquitin ligase ligand via an oxygen, nitrogen, sulfur, carbonyl, or ethynyl of the E3 ubiquitin ligase ligand. In one embodiment, the GPER ligand is an estrogen steroid that includes a divalent group selected from oxygen, amine, sulfur, vinyl, ethyne, and carbonyl, which is positioned at C6, or C17, of the estrogen steroid, and the divalent group at C6 or C17 is attached to the linker. In one embodiment, the E3 ubiquitin ligase ligand is (S,R,S)-AHPC, which is attached to the linker via an amine of the (S,R,S)-AHPC.

Also provided are methods of using the molecule. In one embodiment, a method to prevent, inhibit or treat an endocrine resistant cancer or hormonal therapy resistant cancer in a mammal is provided. The method includes administering to the mammal a composition having an effective amount of a molecule comprising a G-protein coupled estrogen receptor (GPER) ligand coupled to a linker coupled to a ligand for an E3 ligase. In one embodiment, the mammal is a human. In one embodiment, the administration is systemic. In one embodiment, the administration is oral. In one embodiment, the composition is a tablet. In one embodiment, the composition is a liquid. In one embodiment, the composition is injected. In one embodiment, the mammal is a human who is resistant to aromatase inhibitor therapy.

Also provided is a method to prevent, inhibit or treat triple negative breast cancer in a mammal, comprising: administering to the mammal a composition having an effective amount of a molecule comprising a G-protein coupled estrogen receptor (GPER) ligand coupled to a linker coupled to a ligand for an E3 ligase. In one embodiment, the mammal is a human. In one embodiment, the administration is systemic. In one embodiment, the administration is oral. In one embodiment, the GPER ligand is a GPER antagonist.

Further provided is a method to prevent, inhibit or treat a gynecological cancer, e.g., cervical, ovarian or endometrial cancer, in a female mammal, comprising: administering to the mammal a composition having an effective amount of a molecule comprising a G-protein coupled estrogen receptor (GPER) ligand coupled to a linker coupled to a ligand for an E3 ligase. In one embodiment, the mammal is a human. In one embodiment, the administration is systemic. In one embodiment, the administration is oral.

Also provided is a method to prevent, inhibit or treat cancers including prostate cancer or colon cancer, or any cancer that is not hormone dependent, in a mammal, comprising: administering to the mammal a composition having an effective amount of a molecule comprising a G-protein coupled estrogen receptor (GPER) ligand coupled to a linker coupled to a ligand for an E3 ligase. In one embodiment, the mammal is a human. In one embodiment, the administration is systemic. In one embodiment, the administration is oral. In one embodiment, the GPER ligand is a GPER antagonist.

### BRIEF DESCRIPTION OF FIGURES

FIG. 1. Proteolytic targeting of GPER with exemplary PROTAC.

FIG. 2. Structural confirmation of E2-PROTAC, UI-EP001.

FIG. 3. PROTAC prototype UI-EP001 reduces surface GPER. UI-EP001 (100 uM, 16 hr) causes downmodulation of surface recombinant GPER. Results quantitated at right by corrected total red fluorescence measured from 3 microscopic fields.

FIG. 4. PROTAC prototype Ul-EP001 reduces surface GPER. Similar results were obtained for native GPER in her2+ and TNBC breast cancer cells.

FIG. 5. GRER PROTAC for triple negative breast cancer therapy.

FIG. 6. E2β-PROTAC (100 uM) can reduce surface GPER.

FIG. 7. E2β-PROTAC (100 uM) can reduce surface GPER.

FIG. 8. E2β-PROTAC (100 uM) can reduce surface GPER.

FIG. 9. E2β-PROTAC (100 uM) can reduce surface GPER.

FIG. 10. Assessment of the specificity of UI-EP001 downmodulation.

FIG. 11. Restoblue assay.

FIG. 12. Comparing the survivability of HCC 1806 and SKBR3 treated by E₂-PROTAC and partial PROTAC (after 12 hours of treatment).

FIG. 13. Comparing the survivability of HCC 1806 and SKBR3 treated by E₂-PROTAC and partial PROTAC (after 24 hours of treatment).

FIG. 14. Comparing the survivability of HCC 1806 cell lines pre-treated with Estradiol saturation (24 h of treatment).

FIG. 15. Comparing the survivability of SKBR3 cell lines pre-treated with Estradiol saturation (24 h of treatment).

FIG. 16. IC 50 values.

FIG. 17. Mechanism of PROTAC action. The PROTAC links a target protein to E3 ligase causing its polyubiquitinylation and proteasomal degradation.

FIG. 18. E2-PROTAC (UI-EP001) downmodulates GPER in human breast cancer cells. Structure (A) and NMR spectrum (B) of UI-EP001. Human SKBR3 (Her2+) and HCC-1806 (TNBC) breast cancer cells were exposed to vehicle (untreated) or 100 µM of partial-PROTAC or E2p-PROTAC, Ul-EP001 for 16 hours. Cells were immunostained with GPER antibodies for 30 minutes and surface GPER was detected using goat anti-rabbit Alexa 594 IgG (red). Nuclei were countertained with DAPI (blue). (C).HA-GPER or HA-β1AR cells were treated with Ul-EP001 for the indicated times and immunoblotted with HA antibody (D) or immunostained for surface receptor (E). Total corrected red fluorescence (red) (TCRF) of treated and untreated HA-GPER or HA-β1AR cells was measured by subtracting cell background. Significant degradation of GPER was observed in the presence of E2-PROTAC, UI-EP001 n HA-GPER but not HA-β1AR cells (F). Data not shown, no difference in surface GPER expression is measured in cells similarly treated with free 17β-E2.

FIG. 19. Design of a small molecule GPER-PROTAC. 17β-estradiol will be attached via C6- or C-17 of its estrane ring to chemical linkers of varying subunit length to the VHL-derived ubiquitin E3 ligase ligand, (S,R,S) AHPC.

FIG. 20. Synthesis of C-17 linked GPER-PROTAC. Partial PROTACs consisting of (S,R,S)-AHPC-PEG2-COOH; (S,R,S)-AHPCPEG4-COOH or (S,R,S)-AHPC-PEG6-COOH will be coupled via the 17β-OH group of 17β-estradiol by EDC coupling.

FIG. 21. Synthesis of C-6 linked GPER-PROTAC. Partial PROTACs consisting of (S.R.S)-AHPC-PEG2-COOH; (S,R,S)-AHPC-PEG4-COOH or (S,R,S)-AHPC-PEG6-COOH will be coupled to the C-6 carbon atom in several steps diagrammed above.

FIG. 22. Detection of plasma membrane and intracellular GPER by NanoBiT luminescence assay. Intact or detergent-permeabilized. HEK293 cells or HiBiT-GPER- HEK293 cells were labelled with soluble LgBiT protein plus luminescent substrate for 4 minutes at ambient temperature. Luminescence was measured from quadruplicate samples. Background in empty wells containing no cells was subtracted and reported as relative luminescent units (RLU).

FIG. 23. Modeling of PROTACs in GPER and ERα. A) UI-EP001 (blue) and UI-EP002 (green) bound within a GPER homology model, which were designed to have the linker exiting the binding pocket with two varying lengths. B) GPER binding pocket (grey surface) showing the exit of UI-EP001 (blue) and Ul-EP002 (green) from between TM1 and TM7. C) UI-EP001 (blue) and UI-EP002 (green) bound within the ERα ligand domain of an ERα homodimer (PDB:1A52). D) ERα binding pocket (grey surface) showing the exit of UI-EP001 (blue) and UI-EP002 (green).

FIG. 24. Synthesis of UI-EP001 and UI-EP002. Scheme 1: Synthesis of partial PROTAC (compound 7), reagents and conditions: (i) NaH, dioxane, rt, overnight; (ii) TFA/DCM, rt, 2h; (iii) Pd(OAc)_{2,} KOA_{C}, DMAc, 120° C., 24 h; (iv) CoCl₂, NaBH₄, anhydrous methanol, 0° C. to rt, 2 h; (v) HBTU, DIPEA, anhydrous DMF, rt, overnight; (vi) 1. TFA/DCM, rt, 30 min; 2. Boc-Tle-OH, HBTU, DIPEA, anhydrous DMF, rt, overnight; (vii) compound 2, HBTU, DIPEA, rt, overnight. Scheme 2: Synthesis of UI-EP001 (compound 8). Reagents and conditions: (viii) DMAP, EDC, anhydrous DMF, rt, 48 h. Scheme 3: Synthesis of UI-EP002 (compound 10). Reagents and conditions: (ix) Fmoc-NH-PEG₈-CH₂CH₂COOH, DMAP, EDC, anhydrous DMF, rt, 72h; (x) 1. Et₃N, anhydrous DMF; 2. compound 7, HATU, DIPEA, anhydrous DMF, rt, 48 h.

FIG. 25. E2-PROTACs act via high-affinity binding to GPER and ERs. A) Intact HEK-293 (ER⁻, GPER⁻), HA-GPER (ER-, GPER⁺), SKBR3 (ER⁻, GPER⁺) and MCF-7 (ER⁺, GPER⁺) cells were labeled with rabbit GPER antibody at 4° C. Surface GPER was then visualized with Alexa 594-conjugated goat anti-rabbit IgG (red). B) Calculated specific binding as a function of E2-FITC concentration in intact SKBR3 cells. C) Specific binding of 17b-E2, UI-EP001, UI-EP002, and partial PROTAC in intact SKBR3 cells measured by a competitive binding assay using E2-FITC as a fluoro-tracer. D) Determination of E2-FITC total binding using 10 nM E2-FITC in the presence of increasing concentrations of cytosolic protein prepared from MCF-7 cells. E) Fluorescence polarization assay using E2-FITC to assess specific binding of E2, UI-EP001, UI-EP002, and partial PROTAC in cytosolic fractions prepared from MCF-7 cells. Mean values and SDs were derived from three independent experiments.

FIG. 26. E2-PROTACs promote rapid loss of recombinant membrane and intracellular estrogen receptor. HEK-293 cells (ERα⁻, GPER⁻) were transiently transfected with 50 ng HiBiT-GPER or HiBiT-ER plus pcDNA3.1(+) zeo carrier plasmid. Concentration-response curves of (A) HiBiT-GPER and (B) HiBiT-ERα were measured using extracellular LgBiT and luminescence substrate in detergent-permeabilized cells after treatment with increasing doses of either UI-EP001 or UI-EP002 for 1 hour. (C) Histogram depicting total HiBiT-GPER and HiBiT-ER following a 1-hour treatment at 100 mM drug or control. Kinetics of (D) HiBiT-GPER and (E) HiBiT-ERα after incubation of cells with 100 µM of UI-EP001, UI-EP002, and partial PROTAC for various incubation periods from 1 to 8 hours. Results shown represent the mean ± S.E. of three independent experiments. (***, P <0.0004; ****, P <0.0001; one-way ANOVA).

FIG. 27. E2-PROTACs selectively reduce the expression of native and recombinant GPER. (A) HEK-293 cells stably expressing HA-GPER, HA-β1AR, or HA-CXCR4 were treated with 100 µM of Ul-EP001, UI-EP002, and partial PROTAC for 1 hour. Fixed cells were permeabilized and then labeled with rabbit HA antibody and total receptors were then visualized using Alexa Fluor 594 anti-rabbit secondary antibody (red). (B) Corrected Total Red Fluorescence (CTRF) from images of HA-GPER, HA-β1AR, and HA-CXCR4 cells treated with either vehicle, UI-EP001, UI-EP002, and partial PROTAC was measured using Image J software from three different microscopic fields (***, P <0.0004; ****, P <0.0001; one-way ANOVA). (C) MCF-7 (ERa⁺, ERb⁺, PR⁺, GPER⁺) cells were incubated at 37° C. with vehicle, UI-EP001, UI-EP002, or partial PROTAC for 1 hour and then immunostained with mouse ERα, rabbit ERβ, mouse PR, and rabbit GPER antibodies and detected with either Alexa 488-conjugated anti-mouse IgG or Alexa 488-conjugated anti-rabbit IgG (green). (D) Quantification of results from images in (C) measured as CTRF. (E) SKBR3 (ERa⁺, ERb, GPER⁺) cells were incubated with vehicle (1% DMSO), 100 uM of UI-EP001, UI-EP002, or partial PROTAC for 1 hour. Surface and intracellular GPER were visualized in intact or detergent permeabilized cells, respectively using rabbit GPER antibodies and Alexa 594-conjugated goat anti-rabbit IgG (red).

FIG. 28. E2-PROTACs induce proteasome-dependent degradation of GPER and ERα. HEK-293 cells transiently transfected with (A) HiBiT-GPER and (B) HiBiT-ERα were treated with 100 µM UI-EP001 in the presence of increasing concentrations of E2β or aldosterone. Total receptor was then measured by binary luminescence complementation in detergent-permeabilized cells. (C) Effects of 100 µM UI-EP001 alone or in combination with either 100 µM E2 or aldosterone in transiently transfected cells. **** Denotes statistical significance, P < 0.0001 or less. (D) Cells were treated with either (D) 10 µM MG132 or (E) 100 µM chloroquine in the presence or absence of 100 µM of UI-EP001 or UI-EP002 for 1 hour. Total receptor was then quantified by binary luminescence complementation. Representative luminescence data and mean values and SDs derived from three independent experiments are shown. (***, P <0.0004; **, P <0.001; ns, not significant; one-way ANOVA).

FIG. 29. E2-PROTACs induce estrogen receptor-specific cell cytotoxicity in human breast cancer cells. Cell viability of SKBR3, HCC-1806, MCF-7 and MDA-MB-231 breast cancer cells were measured after 24 hour treatment with increasing concentrations of either UI-EP001 or UI-EP002 or partial PROTAC. Results shown represent the mean ± S.E. of three independent experiments.

FIG. 30. E2-PROTACs promote estrogen receptor-dependent G2/M arrest. E2-PROTAC induced G2/M arrest in human breast cancer cell lines expressing membrane and/or intracellular estrogen receptors. SKBR3, HCC-1806, MCF-7 and MDA-MB-231 cells growing in 10% FBS were treated with 10 µM of UI-EP001 or UI-EP002 or partial PROTAC for 24 h. Fixed cells were permeabilized and dyed with propidium iodide (PI). Cell cycle data were acquired on a flow cytometer and analyzed using Flow-Jo software. Data are expressed as mean ± SEM (n=3). The percentage of cells in each phase were plotted using Prism 8.0.

FIG. 31. E2-FITC synthetic scheme.

FIG. 32. Exemplary structures.

FIG. 33. NMR data.

FIG. 34. Gating method for cell cycle analysis. First gate was applied on a scatter plot (A) to gate out debris. As the cells were identified, a second gate was applied to the single cell population using pulse processing (pulse width vs. pulse area) (B) to exclude the cell doublets from analysis. From this cell population, the percentage of each subpopulation in each phase was determined by a histogram plot (C).

FIG. 35. Nude mice challenged with MCF7 (treatment 100 µl IT every 2 days (7x).

FIG. 36. Nude mice challenged with MCF7 (treatment 100 µl IT every 2 days (7x)

FIG. 37. Nude mice challenged with SKB3 (treatment 100 µl IT every 2 days (7x).

FIG. 38. CIMBA-PROTAC degrades recombinant GPER. HEK293 cells stably expressing recombinant HA-tagged GPER were treated with various concentrations of CIMBA-PROTAC-001 or partial PROTAC for 16 hr and then lysed in detergent. Equivalent amounts of protein lysate were resolved in SDS-polyacrylamide gels and then electrotransferred onto PVDF nylon membranes. Membranes were probed with rabbit HA antibodies, top. HA- antibodies were stripped from the membrane at low pH and the membrane was then probed with was stripped with transferrin receptor (TFNR) antibodies as a specificity and loading control.

FIG. 39. Exemplary structures (formula (II) or (III)) of GPER-PROTACs with 2-cyclohexyl-4-isopropyl-N-(4-methoxybenzyl)aniline as a GPER binding agent.

FIG. 40. Exemplary ligands for an E3 ligase.

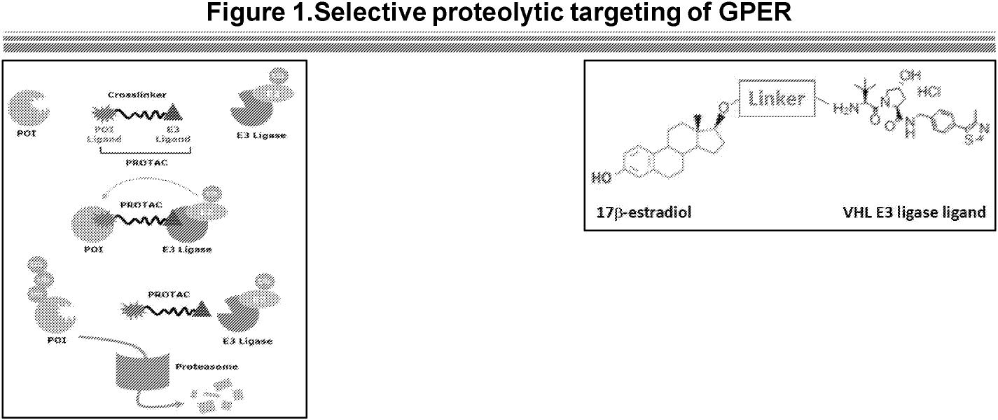

### DETAILED DESCRIPTION

Breast cancer is an estrogen-driven malignancy, and blockade of estrogen action is highly effective for breast cancer and much less toxic than other forms of cancer therapy. However, strategies that inhibit ER function and estrogen biosynthesis are suitable for use only in postmenopausal women with early ER-positive (ER+) breast cancer. Moreover, drug resistance occurs and side effects can be serious (Rugo et al., 2016; Miller et al., 2013). Selective estrogen receptor degraders (SERDs), such as fulvestrant, are effective drugs for the treatment of estrogen receptor (ER)-positive breast cancer, particularly for patients that develop endocrine-resistant disease. SERDs work by binding the ER and inducing allosteric changes that lead to its destruction at the 26S-proteasome. The ER degrader, fulvestrant, is used as second-line therapy for endocrine resistant breast cancer but its bioavailability is poor and thus painful monthly intramuscular injections are required (Nathan et al., 2017).

Estrogen acts via the orphan GPCR homologue, GPR30 (aka GPER), to trigger a Gbg-subunit protein dependent HB-EGF autocrine loop. This finding provided the first mechanistic explanation for the EGF-like effects of estrogen. GPR30 is a Gs-coupled receptor that possesses specific estrogen binding and is linked to adenylyl cyclase. Studies on GPER trafficking demonstrated that it was ubquitinylated at the plasma membrane and employed a clathrin-mediated, b-arrestin- independent retrograde transport to the transGolgi network prior to proteasomal degradation.

Integrin a5b1 is a transmembrane signaling intermediary in GPER-mediated EGFR transactivation. Estrogen action via GPER coordinately promotes fibronectin matrix assembly and EGF-release, cellular actions associated with cell survival. See, e.g., Filardo et al. (2000); Filardo et al. (2002); Pang et al. (2005).

GPER is expressed independently from ER in breast tumor specimens. Moreover, GPER is directly associated with markers of advanced breast cancer, a relationship diametrically opposed to the ER with these same prognostic variables. GPER is also associated with poor prognosis in female reproductive cancer. GPER is upregulated on cortical epithelia during estrus and have collaborated with several groups regarding the role of GPER in neural function within the hypothalamus and luteinizing hormone-releasing. See, e.g., Filardo et al. (2006); Noel et al. (2009); Cheng et al. (2014); and Waters et al. (2015).

Thus, GPER provides an alternative mechanism by which breast cancers respond to estrogen (Ignatov et al., 2011), as the expression of GPER is not limited to ER+ breast cancer (Prissnitz et al., 2015; Neklesa et al., 2017).

A recent survey of 121 patients with TNBC suggests that this receptor is expressed in > 80% of these tumors. Expression of GPER in a majority of TNBC is supported by other smaller studies and is in contrast to data from a rather limited studies of tumors and cell lines suggesting that GPER is a tumor suppressor in TNBC. Unlike the expression of ER, which is inversely associated with clinical predictors of advanced breast cancer, that of GPER associates directly with these same variables, suggesting that it plays a role in metastasis. Thus, the development of therapeutic agents of this type that selectively degrade GPER holds promise for the treatment of TNBC, and may also hold utility for endocrine-resistant breast cancer. Finally, GPER is linked to advanced disease and poor outcome in gynecological cancers, suggesting GPER- PROTACs may also benefit these patients.

### Exemplary GPER-PROTACs

Treatment algorithms for breast cancer are ER-centric and do not yet consider GPER for assignment of estrogen-targeted therapy, despite the fact that GPER is recognized by the International Union of Clinical and Basic Pharmacologists (IUPHAR) (Ignatov et al., 2011). PROTACs are an emerging technique in the development of therapeutic drugs as they eliminate their target, and thereby avoid the selection of drug resistance (Neklesa et al., 2017; Gu et al. 2018). ER-PROTACs show promise in ER-targeted therapy as they show good efficacy and improved bioavailability (Flanagan et al., 2018). ER-PROTACs have been developed for the purpose of targeting ER and consist of synthetic ER alpha-specific ligands (Flanagan et al., 2018; Tria et al., 2018). Thus, they do not effectively target GPER. Synthetic antagonists for GPER exist (Prissnitz et al., 2015) and while they show anti-tumor activity in animal models (Petrie et al., 2013), they are delivered intraperitoneally and chronically and do not overcome the potential development of resistance. A PROTAC approach for targeting GPER is particularly appealing since downmodulation of GPER from the plasma membrane occurs via a ubiquitin-proteasome degradation pathway (Cheng et al., 2011).

ER and GPER bind a diverse group of endogenous, dietary and environmental estrogens (Prissnitz et al., 2015). The relative affinities of these receptors for individual estrogens are distinct, and these affinities cannot be compared because each receptor exists in a different physicochemical environment (Prissnitz et al., 2015; Filardo et al., 2012). Both ER and GPER bind 17alpha-E2 with high affinity. Thus, a GPER-PROTACs having 17alpha-E2 as the GPER-targeting ligand was prepared. The GPER-PROTAC links a target (GPER ligand) to E3 ligase causing GPER polyubiquitinylation and proteasomal degradation.

G-protein coupled estrogen receptor (GPER) is a member of the GPCR superfamily. Following stimulation, GPER undergoes adaptive changes that lead to its desensitization and endocytosis. The 26S proteasome is the natural destination for endocytosed GPER making it well suited for PROTAC-mediated targeting. GPER is directly associated with aggressive disease and poor outcome for breast, endometrial, and ovarian cancer. GPER is expressed in 97 of 121 (> 80%) triple negative breast cancers (TNBCs), making this breast cancer subtype, which has no known therapeutic target, a cancer that is ideally suited for GPER-PROTAC therapy. Available ER-PROTACs do not target GPER as they have been optimized for binding to ERalpha-specific ligands. As described herein, a GPER- PROTAC UI-EP001 and UI-EP00 promoted downmodulation of GPER in human breast cancer cells. GPER-PROTACs with modifications in the composition and length of its chemical spacer can be tested using a competitive radioreceptor binding assay with [³H]-17β-estradiol (17- β-E2) for GPER specific binding.

PROTACs with the highest relative binding affinity (RBA) for 17 β-E2 were assayed for their capacity to promote GPER degradation in breast cancer cells using quantitative immunofluorescent and immunochemical assays. Nanobit™, an exquisitively sensitive binary bioluminescent complementation assay, may be used to confirm GPER elimination. Polyubiquitinylation of GPER is confirmed by Tandem Ubiquitin Binding Element (TUBE) assay, and proteasome-specific degradation is determined using the proteasomal inhibitor, MG132. The capacity of GPER to transactivate the erbB1-to-erk signaling axis is also assessed.

The anti-oncogenic efficacy, biodistribution and toxicity of GPER-PROTACs are tested based on the capacity to delay TNBC recurrence and metastasis using a BRCA-1 mutant mouse that has been widely used as a model of TNBC oncogenesis. Orally-delivered, small molecule PROTACs capable of inactivating GPER in breast cancer are identified. Those small molecules re useful to treat other cancers where inactivation of GPER is indicated, e.g., ovarian cancer, cervical cancer, endometrial cancer, prostate cancer or colon cancer.

### Exemplary Formulations

The disclosed GPER-PROTAC may be delivered in a biodegradable particle that may include or may be formed from biodegradable polymeric molecules which may include, but are not limited to polylactic acid (PLA), polyglycolic acid (PGA), co-polymers of PLA and PGA (i.e., polylactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, polyalkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The particles may be prepared by methods known in the art. (See, e.g., Nagavarma et al., Asian J. of Pharma. And Clin. Res., Vol 5, Suppl 3, 2012, pages 16-23; Cismaru et al., Rev. Room. Chim., 2010, 55(8), 433-442: and International Application Publication Nos. WO 2012/115806; and WO 2012/054425; the contents of which are incorporated herein by reference in their entireties). Suitable methods for preparing the particles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the particles may be prepared by forming a double emulsion (e.g., water-in-oil-in-water) and subsequently performing solvent-evaporation. The particles obtained by the methods may be subjected to further processing steps such as washing and lyophilization, as desired. Optionally, the particles may be combined with a preservative (e.g., trehalose).

Typically, the particles have a mean effective diameter of less than 1 micron, e.g., the particles have a mean effective diameter of between about 25 nm and about 500 nm, e.g., between about 50 nm and about 250 nm, about 100 nm to about 150 nm, or about 450 nm to 650 nm, or a mean effective diameter of between about 25 µm and about 500 µm, e.g., between about 50 µm and about 250 µm, about 100 µm to about 150 µm, or about 450 µm to 650 µm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, a delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to liposomes, emulsions, DOTAP, DOTMA, DMRIE, DOSPA, distearoylphosphatidylcholine (DSPC), DOPE, or DC-cholesterol; peptide based vectors including but not limited to Poly-L-lysine or protamine: or poly(β-amino ester), chitosan, PEI-polyethylene glycol, PEI-mannose-dextrose, or DOTAP-cholesterol.

In one embodiment, the delivery vehicle is a glycopolymer-based delivery vehicle, poly(glycoamidoamine)s (PGAAs). These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units maybe employed.

In one embodiment, the delivery vehicle for the GPER-PROTAC comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The polymer may include, but is not limited to, polyamidoamine (PAMAM) dendrimers. Polyamidoamine dendrimers suitable for preparing the presently disclosed nanoparticles may include 3rd-, 4th-, 5th-, or at least 6th-generation dendrimers.

In one embodiment, the delivery vehicle comprises a lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N-(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N- dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEl-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other materials, for example, PEI, DOTMA, DC-Chol, or CTAB, may be used.

In one embodiment, GPER-PROTACs are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

Numerous liposome delivery systems may be used in formuations having the GPER-PROTAC. Virtually any lipid may be used to form a liposome. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid bi-layer of protocells according to an embodiment Often cholesterol is incorporated to enhance structural integrity of the bi-layer. These lipids are all readily available commercially from Avanti Polar Lipids, Inc. (Alabaster, Alabama, USA).

### Pharmaceutical Compositions

The disclosure provides a composition comprising, consisting essentially of, or consisting of at least one GPER-PROTAC and a pharmaceutically acceptable (e.g., physiologically acceptable) carrier. In one embodiment, when the composition consists essentially of at least one GPER-PROTAC and optionally a pharmaceutically acceptable carrier, additional components can be included that do not materially affect the composition (e.g., adjuvants, buffers, stabilizers, anti-inflammatory agents, solubilizers, preservatives, etc.). In one embodiment, when the composition consists of at least one GPER-PROTAC encapsulated in particles, and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the GPER-PROTAC is administered in a composition formulated to protect the GPER-PROTAC from damage prior to administration. For example, the composition can be formulated to reduce loss of the GPER-PROTAC on devices used to prepare, store, or administer the GPER-PROTAC, such as glassware, syringes, or needles. The composition can be formulated to decrease the light sensitivity and/or temperature sensitivity of the GPER-PROTAC. To this end, the composition may comprise a pharmaceutically acceptable liquid carrier, such as, for example, those described above, and a stabilizing agent selected from the group consisting of polysorbate 80, L-arginine, polyvinylpyrrolidone, trehalose, and combinations thereof.

The composition also can be formulated to enhance transduction efficiency. In addition, one of ordinary skill in the art will appreciate that the at least one GPER-PROTAC can be present in a composition with other therapeutic or biologically-active agents. For example, factors that control inflammation, such as ibuprofen or steroids, can be part of the composition to reduce swelling and inflammation. Immune system stimulators or adjuvants, e.g., interleukins, lipopolysaccharide, and doublestranded RNA may also be administered. Antibiotics, i.e., microbicides and fungicides, can be present to treat existing infection and/or reduce the risk of future infection.

Injectable depot forms are made by forming at least one GPER-PROTAC in biodegradable polymers such as polylactide-polyglycolide. Depending on the ratio of GPER-PROTAC to polymer, and the nature of the particular polymer employed, the rate of GPER-PROTAC release can be controlled. Examples of other biodegradable polymers include poly(orthoesters) and poly(anhydrides). Depot injectable formulations are also prepared by entrapping the GPER-PROTAC optionally in a complex with a polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the GPER-PROTAC. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the GPER-PROTAC in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the GPER-PROTAC described herein. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the GPER-PROTAC to elicit a desired response in the individual.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of at least one GPER-PROTAC.

### Routes of Administration, Dosages and Dosage Forms

Administration of the GPER-PROTAC may be continuous or intermittent, depending, for example, upon the recipient’s physiological condition, and other factors known to skilled practitioners. The administration of the GPER-PROTAC may be essentially continuous over a preselected period of time or may be in a series of spaced doses. Both local administration, e.g., intranasal or intrathecal, and systemic administration are contemplated. Any route of administration may be employed, e.g., intravenous, intranasal or oral, or local administration.

One or more suitable unit dosage forms comprising the GPER-PROTAC, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of GPER-PROTAC administered to achieve a particular outcome will vary depending on various factors including, but not limited to, the condition, patient specific parameters, e.g., height, weight and age, and whether prevention or treatment, is to be achieved.

GPER-PROTACs may conveniently be provided in the form of formulations suitable for administration. A suitable administration format may best be determined by a medical practitioner for each patient individually, according to standard procedures. Suitable pharmaceutical acceptable carriers and their formulation are described in standard formulations treatises, e.g., Remington’s Pharmaceuticals Sciences. By “pharmaceutically acceptable” it is meant a carrier, diluent, excipient, and/or salt that is compatible with the other ingredients of the formulation, and not deleterious to the recipient thereof.

GPER-PROTACs may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The GPER-PROTAC can be provided in a dosage form containing an amount effective in one or multiple doses. The GPER-PROTAC may be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of GPER-PROTAC can be administered.

By way of illustration, liposomes and other lipid-containing complexes can be used to deliver one or more GPER-PROTACs.

Pharmaceutical formulations containing the GPER-PROTAC can be prepared by procedures known in the art using well known and readily available ingredients. For example, the agent can be formulated with common excipients, diluents, or carriers, and formed into tablets, capsules, suspensions, powders, and the like. The GPER-PROTAC can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the vectors may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the GPER-PROTAC may be administered via nose drops, a liquid spray, such as via a plastic bottle atomizer or metered-dose inhaler. Typical of atomizers are the Mistometer (Wintrop) and the Medihaler (Riker).

The local delivery can also be by a variety of techniques which administer the GPER-PROTAC at or near the site of disease, e.g., using a catheter or needle Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, including non-human primates, sheep, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### Exemplary Linkers

Here the terms “linking group,” “linker molecule,” “linker,” and the like refer to any molecular group useful for linking at least two distinct chemical entities. In order to perform the linkage between the chemical entities, it is necessary that each of the reactants contain a chemically complementary reactive group. Examples of complementary reactive groups are amino and carboxyl groups to form amide linkages, carboxy and hydroxy groups to form ester linkages, amino and alkyl halides to form alkylamino linkages, thiols and thiols to form disulfides, or thiols and maleimides or alkylhalides to form thioethers. Hydroxyl, carboxyl, amino, and other functionalities may be introduced by known methods when not already present. If desired, one or more of reactive complementary groups can be “protected”, in which event the protected reactive group must be “deprotected” prior to performing the chemistry needed to effect the particular linkage chemistry. Any suitable protection/deprotection scheme can be employed in a particular circumstance. As those in the art will appreciate, any suitable molecular group can be used as a linker, which molecular group is suitable for a particular situation may vary, although it is easily within the skill of those in the art to select or prepare an appropriate molecular group with suitable chemically complementary reactive groups to perform the desired linkage. Regardless of the molecular group selected in a particular circumstance, it may provides for stable covalent linkage between the different chemical entities to form a conjugate according to the invention. Specifically, a covalent linkage should be stable relative to the solution conditions under which the linker and linking groups are subjected. Generally, linkers of any suitable length or arrangement can be employed, although linkers that contain about 4 to 80 carbons, preferably from about 10 to 70 carbons, about 10 to 50 carbons, or from about 10 to 30 carbons or about 10 to 20 carbons, are envisioned. Linkers may also contain one or more heteroatoms (e.g., N, O, S, and P) in the molecular linking groups, particularly from 0 to 10 heteroatoms. In various embodiments, the linker can contain 3, 4, 5, 6, 7, 8, 9, 10, 11, 12, 13, 14, 15, 16, 17, 18, 19, or 20 heteroatoms. In yet further embodiments, the linker contains 3, 4, 5, 6, 7, 8, 9, 10, 11, 12, 13, 14, 15, 16, 17, 18, 19, or 20 divalent oxygen (—O—), divalent nitrogen (—NH—), or both. The molecular linking group may be branched or straight chain. It will also be appreciated that in some cases, conjugates may be formed directly between a purine analog and targeting moiety or specific binding molecule, in which case a linker is not employed. In such cases, a substituent of the purine analog and a substituent of the specific binding molecule are typically derivatized to provide the complementary reactive groups (one or more which may, if appropriate, be protected) necessary to perform a suitable chemistry to link the different chemical entities.

The length of linker chain, in terms of atoms, can be determined based on counting the number of atoms along a linear backbone of the linker, from the G-protein coupled estrogen receptor (GPER) ligand to the E3 ubiquitin ligase ligand, but not including an atom that corresponds to an atom in an unlinked GPER ligand or E3 ubiquitin ligase ligand. For example, if beta-estradiol is the GPER ligand, then when counting the number of atoms in the linker chain, neither oxygen of beta-estradiol would be counted. In yet further embodiments, the linker has a chain length of having a number of atoms in the backbone about, equal to, or greater than, 5, 6, 7, 8, 9, 19, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, up to 200 atoms, as counted in a linear path between, but not including, the GPER ligand and the E3 ubiquitin ligase ligand. In further embodiments, the chain length in terms of atoms of less than or equal to 6, 7, 8, 9, 19, 11, 12, 13, 14, 15, 20, 30, 40, 50, 60, 70, 80, 90, or 100 atoms. The length of the linker can also be described in terms of angstroms based on calculating bond lengths in a linear path between the GPER ligand and the E3 ubiquitin ligase ligand, including the bonds connecting the linker to each such ligand. In various further embodiments, the linker can have such a calculated length of about, equal to, or greater than, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 angstroms, up to 300 angstroms, as calculated based on adding bond lengths in a linear path between the GPER ligand and the E3 ubiquitin ligase ligand. In further embodiments, the linker has a calculated length of less than or equal to 10, 15, 20, 25, 30, 35, 40, 45, 50, 60, 70, 80, 90, or 100 angstroms.

Non-limiting examples of linkers useful in GPER-PROTACs include an oxygen atom, a sulfur atom, a nitrogen atom and/or a carbon atom (and appropriately appended hydrogen atoms when necessary to fill valences) but also linkers with side chains that increase solubility, such as, for example, groups containing morpholino, piperidino, pyrrolidino, or piperazino rings and the like; amino acids, polymers of amino acids (proteins or peptides), e.g., dipeptides or tripeptides, and the like; carbohydrates (polysaccharides), nucleotides such as, for example, PNA, RNA and DNA, and the like; polymers of organic materials, such as, for example, polyethylene glycol, polylactide and the like. In one embodiment, the linker can be a divalent aryl or heteroaryl, bis-amide aryl, bis-amide heteroaryl, bis-hydrazide aryl, bis-hydrazide heteroaryl, or the like. In one embodiment, the linker has a chain having up to about 24 to 50 atoms; wherein the atoms are selected from the group consisting of carbon, nitrogen, sulfur, non-peroxide oxygen, and phosphorous. In one embodiment, the linker has a chain having from about 4 to about 12 to 20 atoms or about 16 to about 48 atoms.

In one embodiment, the linker is an alkylene linker, phenylene linker, or an alkyl linker. The linker may be C₄-C₄₈ alkyl or a hetero form thereof. These linkers may include a carbonyl group. In certain embodiments, a linker sometimes is a —C(Y′)(Z′)—C(Y″)(Z″)— linker, where each Y′, Y″, Z′ and Z″ independently is hydrogen C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C1-6 alkoxy C₁₋₆ alkenyl, amino, cyano, halogen or aryl. In one embodiment, the linker is a chain wherein the atoms of the chain are selected from the group consisting of carbon, nitrogen, sulfur, and oxygen, wherein any carbon atom can be substituted with, e.g., oxo, and wherein any sulfur atom can be substituted with one or two oxo groups. The chain may be interspersed with one or more cycloalkyl, aryl, heterocyclyl, or heteroaryl rings. In one embodiment, the linker comprises a chain having about 4 to about 50 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups.

In one embodiment, the linker includes —(Y)_{y}—, —(Y)_{y}—C(O)N—(Z)_{z}—, —(CH₂)_{y}—C(O)N—(CH₂)_{z}—, —(Y)_{y}—NC(O)—(Z)_{z}—, —(CH₂)_{y}—NC(O)—(CH₂)_{z}—, where each y (subscript) and z (subscript) independently is 0 to 20 and each Y and Z independently is C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C₁₋₆ alkoxy C₁₋₆ alkenyl, amino, cyano, halogen or aryl. In certain embodiments, a linker sometimes is a —C(Y′)(Z′)—C(Y″)(Z″)— linker, where each Y′, Y″, Z′ and Z″ independently is hydrogen C₁-C₁₀ alkyl, substituted C₁-C₁₀ alkyl, C₁-C₁₀ alkoxy, substituted C₁-C₁₀ alkoxy, C₃-C₉ cycloalkyl, substituted C₃-C₉ cycloalkyl, C₅-C₁₀ aryl, substituted C₅-C₁₀ aryl, C₅-C₉ heterocyclic, substituted C₅-C₉ heterocyclic, C₁-C₆ alkanoyl, Het, Het C₁-C₆ alkyl, or C₁-C₆ alkoxycarbonyl, wherein the substituents on the alkyl, cycloalkyl, alkanoyl, alkcoxycarbonyl, Het, aryl or heterocyclic groups are hydroxyl, C₁-C₁₀ alkyl, hydroxyl C₁-C₁₀ alkylene, C₁-C₆ alkoxy, C₃-C₉ cycloalkyl, C₅-C₉ heterocyclic, C1-6 alkoxy C₁₋₆ alkenyl, amino, cyano, halogen or aryl

In one embodiment, the linker comprises an amido linking group (e.g., —C(O)NH— or—NH(O)C—); alkyl amido linking group (e.g., -C₁-C₆ alkyl—C(O)NH—, -C₁-C₆ alkyl—NH(O)C—, —C(O)NH—C₁-C₆ alkyl-, —NH(O)C—C₁-C₆ alkyl-, -C₁-C₆ alkyl--NH(O)C-C₁-C₆ alkyl-, -C₁-C₆ alkyl-C(O)NH-C₁-C₆ alkyl-, or—C(O)NH—(CH₂)ₜ—, where t is 1, 2, 3, or 4); substituted 5-6 membered ring (e.g., aryl ring, heteroaryl ring (e.g., tetrazole, pyridyl, 2,5-pyrrolidinedione (e.g., 2,5-pyrrolidinedione substituted with a substituted phenyl moiety)), carbocyclic ring, or heterocyclic ring) or oxygen-containing moiety (e.g., —O—, -C₁-C₆ alkoxy).

In one embodiment, the linker comprises a polyethylene glycol (PEG) moiety (R³)ᵣwherein R³ is a PEG unit and r is about 1 to about 10 (e.g., r is about 2 to about 6). In certain embodiment R³ is —O—CH₂—CH₂— or —CH₂—CH₂—O—. In some embodiments R³ is —O—CH₂—CH₂— or —CH₂—CH₂—O— and r is about 1 to about 100 (e.g., about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, or 100. In certain related embodiments, r is about 5 to about 25, about 10 to about 50, about 5 to about 15, about 12 to about 35, about 25 to about 55 or about 65 to about 95. In some embodiments the (R³)ᵣ substituent is linear, and in certain embodiments, the (R³)ᵣ substituent is branched. For linear moieties, s sometimes is less than r (e.g., when R³ is —O—CH₂—CH₂— or —CH₂—CH₂—O—) and at times s is 1. In some embodiments R³ is a linear PEG moiety (e.g., having about 1 to about 30 PEG units).

Other linkers include but are not limited to those in WO19/123367 and Sun et al. (Signal Transd. Targeted Ther., 4:64 (2019)), the disclosures of which are incorporated by reference herein

As used herein, the terms “alkyl,” “alkenyl” and “alkynyl” may include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2 propenyl, 3 butynyl, and the like. The total number of carbon atoms in each such group is sometimes described herein, e.g., when the group can contain up to ten carbon atoms it can be represented as 1-10C or as C₁-C₁₀ or C₁₋₁₀. When heteroatoms (N, O and S typically) are allowed to replace carbon atoms as in heteroalkyl groups, for example, the numbers describing the group, though still written as e.g. C₁-C₆, represent the sum of the number of carbon atoms in the group plus the number of such heteroatoms that are included as replacements for carbon atoms in the backbone of the ring or chain being described.

Typically, the alkyl, alkenyl and alkynyl substituents of the invention contain one 10C (alkyl) or two 10C (alkenyl or alkynyl). For example, they contain one 8C (alkyl) or two 8C (alkenyl or alkynyl). Sometimes they contain one 4C (alkyl) or two 4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often optionally substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C₁-C₈ alkyl, C₂-C₈ heteroalkyl, C₁-C₈ acyl, C₂-C₈ heteroacyl, C₂-C₈ alkenyl, C₂-C₈ heteroalkenyl, C₂-C₈ alkynyl, C₂-C₈ heteroalkynyl, C₆-C₁₀ aryl, or C₅-C₁₀ heteroaryl, and each R is optionally substituted with halo, ═O, ═N—CN, ═N—OR′, ═NR′, OR′, NR′₂, SR’, SO₂R′, SO₂NR′₂, NR′SO₂R′, NR′CONR′₂, NR′COOR′, NR′COR′, CN, COOR’, CONR′₂, OOCR′, COR′, and NO₂, wherein each R′ is independently H, C₁-C₈ alkyl, C₂-C₈ heteroalkyl, C₁-C₈ acyl, C₂-C₈ heteroacyl, C₆-C₁₀ aryl or C₅-C₁₀ heteroaryl. Alkyl, alkenyl and alkynyl groups can also be substituted by C₁-C₈ acyl, C₂-C₈ heteroacyl, C₆-C₁₀ aryl or C₅-C₁₀ heteroaryl, each of which can be substituted by the substituents that are appropriate for the particular group.

“Acetylene” substituents may include 2-10C alkynyl groups that are optionally substituted, and are of the formula —C≡C—Ri, wherein Ri is H or C₁-C₈ alkyl, C₂-C₈ heteroalkyl, C₂-C₈ alkenyl, C₂-C₈ heteroalkenyl, C₂-C₈ alkynyl, C₂-C₈ heteroalkynyl, C₁-C₃ acyl, C₂-C₈ heteroacyl, C₆-C₁₀ aryl, C₅-C₁₀ heteroaryl, C₇-C₁₂ arylalkyl, or C₆-C₁₂ heteroarylalkyl, and each Ri group is optionally substituted with one or more substituents selected from halo, ═O, ═N—CN, ═N—OR′, ═NR′, OR′, NR′2, SR′, SO₂R′, SO₂NR′₂, NR′SO₂R′, NR′CONR′₂, NR′COOR′, NR′COR′, CN, COOR′, CONR′₂, OOCR′, COR, and NO₂, wherein each R′ is independently H, C₁-C₆ alkyl, C₂-C₆ heteroalkyl, C₁-C₆ acyl, C₂-C₆ heteroacyl, C₆-C₁₀ aryl, C₅-C₁₀ heteroaryl, C₇₋₁₂ arylalkyl, or C₆₋₁₂ heteroarylalkyl, each of which is optionally substituted with one or more groups selected from halo, C₁-C₄ alkyl, C₁-C₄ heteroalkyl, C₁-C₆ acyl, C₁-C₆ heteroacyl, hydroxy, amino, and ═O; and wherein two R′ can be linked to form a 3-7 membered ring optionally containing up to three heteroatoms selected from N, O and S. In some embodiments, Ri of —C≡C—Ri is H or Me.

“Heteroalkyl”, “heteroalkenyl”, and “heteroalkynyl” and the like are defined similarly to the corresponding hydrocarbyl (alkyl, alkenyl and alkynyl) groups, but the ‘hetero’ terms refer to groups that contain one to three O, S or N heteroatoms or combinations thereof within the backbone residue; thus at least one carbon atom of a corresponding alkyl, alkenyl, or alkynyl group is replaced by one of the specified heteroatoms to form a heteroalkyl, heteroalkenyl, or heteroalkynyl group. The typical sizes for heteroforms of alkyl, alkenyl and alkynyl groups are generally the same as for the corresponding hydrocarbyl groups, and the substituents that may be present on the heteroforms are the same as those described above for the hydrocarbyl groups. For reasons of chemical stability, it is also understood that, unless otherwise specified, such groups do not include more than two contiguous heteroatoms except where an oxo group is present on N or S as in a nitro or sulfonyl group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)—heteroaryl.

Acyl and heteroacyl groups are bonded to any group or molecule to which they are attached through the open valence of the carbonyl carbon atom. Typically, they are C₁-C₈ acyl groups, which include formyl, acetyl, pivaloyl, and benzoyl, and C₂-C₈ heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5 membered rings as well as 6 membered rings. Typical heteroaromatic systems include monocyclic C₅-C₆ aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C₈-C₁₀ bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. For example, the monocyclic heteroaryls may contain 5-6 ring members, and the bicyclic heteroaryls contain 8-10 ring members.

Aryl and heteroaryl moieties may be substituted with a variety of substituents including C₁-C₈ alkyl, C₂-C₈ alkenyl, C₂-C₈ alkynyl, C₅-C₁₂ aryl, C₁-C₈ acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C₁-C₈ alkyl, C₂-C₈ heteroalkyl, C₂-C₈ alkenyl, C₂-C₈ heteroalkenyl, C₂-C₈ alkynyl, C₂-C₈ heteroalkynyl, C₆-C₁₀ aryl, C₅-C₁₀ heteroaryl, C₇-C₁₂ arylalkyl, or C₆-C₁₂ heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C₁-C₈ alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. For example, an arylalkyl group includes a phenyl ring optionally substituted with the groups defined above for aryl groups and a C₁-C₄ alkylene that is unsubstituted or is substituted with one or two C₁-C₄ alkyl groups or heteroalkyl groups, where the alkyl or heteroalkyl groups can optionally cyclize to form a ring such as cyclopropane, dioxolane, or oxacyclopentane. Similarly, a heteroarylalkyl group may include a C₅-C₆ monocyclic heteroaryl group that is optionally substituted with the groups described above as substituents typical on aryl groups and a C₁-C₄ alkylene that is unsubstituted or is substituted with one or two C₁-C₄ alkyl groups or heteroalkyl groups, or it includes an optionally substituted phenyl ring or C₅-C₆ monocyclic heteroaryl and a C₁-C₄ heteroalkylene that is unsubstituted or is substituted with one or two C₁-C₄ alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus, a benzyl group is a C₇-arylalkyl group, and phenylethyl is a Cs-arylalkyl.

“Heteroarylalkyl” as described above refers to a moiety comprising an aryl group that is attached through a linking group, and differs from “arylalkyl” in that at least one ring atom of the aryl moiety or one atom in the linking group is a heteroatom selected from N, O and S. The heteroarylalkyl groups are described herein according to the total number of atoms in the ring and linker combined, and they include aryl groups linked through a heteroalkyl linker; heteroaryl groups linked through a hydrocarbyl linker such as an alkylene; and heteroaryl groups linked through a heteroalkyl linker. Thus, for example, C₇-heteroarylalkyl would include pyridylmethyl, phenoxy, and N-pyrrolylmethoxy.

“Alkylene” as used herein refers to a divalent hydrocarbyl group; because it is divalent, it can link two other groups together. Typically it refers to —(CH₂)ₙ— where n is 1-8 and for instance n is 1-4, though where specified, an alkylene can also be substituted by other groups, and can be of other lengths, and the open valences need not be at opposite ends of a chain. Thus —CH(Me)— and —C(Me)₂— may also be referred to as alkylenes, as can a cyclic group such as cyclopropan-1,1-diyl. Where an alkylene group is substituted, the substituents include those typically present on alkyl groups as described herein.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of a linker R² is alkyl, this alkyl may optionally be substituted by the remaining substituents listed as embodiments for R² where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, =O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

In one embodiment, the linker comprises (R³),,- (R⁴) or is R³ or is ((R³)ᵣ₋(R⁴)-(R³), In one embodiment, R³ is a PEG moiety or a derivative of a PEG moiety. In certain embodiment R³ is —O—CH₂—CH₂— or —CH₂—CH₂—O—. In one embodiment, a PEG moiety can include one or more PEG units. A PEG moiety can include about 1 to about 1,000 PEG units, including, without limitation, about 1, 2, 3, 4, 5, 6, 7, 8, 9, 10, 11, 12, 13, 14, 15, 16, 17, 18, 19, 20, 21, 22, 23, 24, 25, 30, 40, 50, 60, 70, 80, 90, 100, 200, 300, 400, 500, 600, 700, 800 or 900 units, in some embodiments. In certain embodiments, a PEG moiety can contain about 1 to 5 and up to about 25 PEG units, about 1 to 5 up to about 10 PEG units, about 10 up to about 50 PEG units, about 18 up to about 50 PEG units, about 47 up to about 150 PEG units, about 114 up to about 350 PEG units, about 271 up to about 550 PEG units, about 472 up to about 950 PEG units, about 50 up to about 150 PEG units, about 120 up to about 350 PEG units, about 250 up to about 550 PEG units or about 650 up to about 950 PEG units. A PEG unit is —O—CH₂—CH₂— or —CH₂—CH₂—O— in certain embodiments. In one embodiment, R⁴ is NH(CO), —C₁—C₆, alkyl, -C₁-C₆ alkoxy, —NR^{a}R^{b}, —N₃, —OH, —CN, —COOH, —COOR¹, —C₁—C₆ alkyl-NR^{a}R^{b}, C₁-C_{6,} alkyl-OH, C₁-C₆, alkyl-CN, C₁,—C₆ alkyl-COOH, C₁-C₆ alkyl-COOR¹, 5-6 membered ring, substituted 5-6 membered ring, —C₁—C₆ alkyl- 5-6 membered ring, -C₁-C₆ alkyl- substituted 5-6 membered ring C₂-C₉ heterocyclic, or substituted C₂-C₉ heterocyclic

In some embodiments R³ is —O—CH₂—CH₂— or —CH₂—CH₂—O— and r or t_{;} independently is 1, 2, 3, 4, 5, 6, 7, 8, 9, 10, 11, 12, 13, 14, 15, 16, 17, 18, 19, or 20. In certain embodiments, r or t is about 4 to about 15 and sometimes r is about 4 or 11. In some embodiments, R³ is a PEG unit (PEG)ᵣ and r is about 2 to about 4 and t is about 8 to 14.

In certain embodiments, R₄ is an amido linking group (e.g., —C(O)NH— or —NH(O)C—); alkyl amido linking group (e.g., —C₁—C₆ alkyl—C(O)NH—, —C₁—C₆ alkyl—NH(O)C—, —C(O)NH—C₁-C₅ alkyl-, —NH(O)C—C₁-C₆ alkyl-, —C₁—C₆ alkyl-NH(O)C-C₁-C₆ alkyl-, —C₁—C₆ alkyl-C(O)NH-C₁-C₆ alkyl-, or —C(O)NH—(CH₂)t—, where t is 1, 2. 3, or 4); substituted 5-6 membered ring (e.g., aryl ring, heteroaryl ring (e.g., tetrazole, pyridyl, 2,5-pyrrolidinedione (e.g., 2,5-pyrrolidinedione substituted with a substituted phenyl moiety)), carbocyclic ring, or heterocyclic ring) or oxygen-containing moiety (e.g., —O—, —C₁—C₆ alkoxy).

### Exemplary Embodiments

In one embodiment, a molecule comprising a G-protein coupled estrogen receptor (GPER) ligand coupled to a linker coupled to a ligand for an E3 ligase is provided. In one embodiment, the GPER ligand comprises 17β-estradiol, estrone, a phytoestrogen, a xenoestrogen, estriol, estriol 3-sulfate, estriol 17-sulfate, G-1, G-15, G-36, genistein or quercetin. In one embodiment, the GPER ligand comprises 17β-estradiol. In one embodiment, the GPER ligand is a GPER antagonist. In one embodiment, the E3 ligase ligand is a Von Hippel ligase (VHL) ligand. In one embodiment, the E3 ligase ligand comprises lenalidomide, pomalidomide, iberdomide, (S,R,S)-AHPC, thalidomide, VH-298, CC-885, E3ligase ligand 8, TD-106, VL285, VH032, VH101,VH298, VHL ligand 4, VHL-2 ligand 3, E3 ligase ligand 3, E3 ligase ligand 2, or BC-1215. In one embodiment, the linker has a chain having 5 to 50 atoms, e.g., from about 8 to about 35 atoms. In one embodiment, the linker is an alkyl linker. In one embodiment, the linker is a heteroalkyl linker having one or more O, N or S. In one embodiment, the linker comprises polyethylene glycol (PEG). In one embodiment, the linker comprises 4 to 15 PEG units. In one embodiment, the linker comprises (PEG)ₘNH(CO)(PEG)ₙ where n and m independently are 0, 1, 2, 3, 4, 5, 6, 7, 8, 9, 10, 11, 12, 13, 14 or 15. In one embodiment, n is 3, 4, 5, or 6. In one embodiment, m is 7, 8, 9, or 10. In one embodiment, the GPER ligand, the linker or the E3 ligand comprises an amine group, a carboxyl group, a carbonyl group, a sulfhydryl group, e.g., maleimide, an aldehyde group, e.g., hydrazide, or a hydroxyl group useful for forming a covalent bond with another molecule. In one embodiment, the linker comprises a carbodiimide, e.g., EDC, NHS, ABH, ANB-NOS, APDP, EMCH, EMCS, GMBS, MBS, or SlAB.

In one embodiment, a method to prevent, inhibit or treat an endocrine resistant cancer or a hormonal therapy resistant cancer in a human is provided. In one embodiment, the method includes administering to the human a composition having an effective amount of a molecule comprising a G-protein coupled estrogen receptor (GPER) ligand coupled to a linker coupled to a ligand for an E3 ligase.

In one embodiment, a method to prevent, inhibit or treat triple negative breast cancer in a human is provided comprising administering to the mammal a composition having an effective amount of a molecule comprising a G-protein coupled estrogen receptor (GPER) ligand coupled to a linker coupled to a ligand for an E3 ligase.

In one embodiment, a method to prevent, inhibit or treat cervical, ovarian or endometrial cancer in a human is provided that includes administering to the mammal a composition having an effective amount of a molecule comprising a G-protein coupled estrogen receptor (GPER) ligand coupled to a linker coupled to a ligand for an E3 ligase.

In one embodiment, a method to prevent, inhibit or treat prostate or ovarian cancer in a human is provided that includes administering to the mammal a composition having an effective amount of a molecule comprising a G-protein coupled estrogen receptor (GPER) ligand coupled to a linker coupled to a ligand for an E3 ligase.

### Exemplary Chimeras

A compound is provided having a structure of Formula I, or a salt thereof,

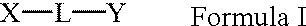

wherein
X is a G-protein coupled estrogen receptor (GPER) ligand;
Y is an E3 ubiquitin ligase ligand; and
L is a linker.

In one example, X is 17β-estradiol, estrone, a phytoestrogen, a xenoestrogen, estriol, estriol 3-sulfate, estriol 17-sulfate, G-1, G-15, G-36, genistein, dazine, quercetin, or a derivative thereof. In one example, X is a GPER antagonist. In one example, Y is a Von Hippel ligase (VHL) ligand. In one example, Y is cereblon, lenalidomide, pomalidomide, iberdomide, (S,R,S)-AHPC, thalidomide, VH-298, CC-122, CC-885, E3ligase ligand 8, TD-106, VL285, VH032, VH101, VH298, VHL ligand 4, VHL ligand 7, VHL-2 ligand 3, E3 ligase ligand 3, E3 ligase ligand 2, BC-1215, or a derivative thereof. In one example, L has a backbone having a chain length of 5 to 200 atoms as counted in a linear path between X and Y. In one example, L has a backbone having a chain length of 15 to 50 atoms as counted in a linear path between X and Y. In one example, L has a backbone having a chain length of 20 to 50 atoms as counted in a linear path between X and Y. In one example, L has a backbone having a calculated length of 8 to 300 angstroms as determined from summing bond lengths in a linear path between X and Y. In one example, L has a backbone having a calculated length of 25 to 75 angstroms as determined from summing bond lengths in a linear path between X and Y. In one example, L has the structure:

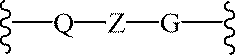

wherein
Q is a bond or a divalent group that forms a covalent linkage to X;
Z is a linear chain comprising one or more alkyl, aryl, heteroalkyl, heteroaryl, alkyloxy, alkylamino, alkylglycol, carbonyl, thiocarbonyl, acyl, carbamate, urea, thiocarbamate, thiourea, dithiocarbamate, aminocarbonyl, amide, ester, thioester, thioamide, amine, oxygen, sulfur, sulfone, or sulfoxide, in divalent form;
G is a bond or a divalent group that forms a covalent linkage to Y.

In one example, L has the structure:

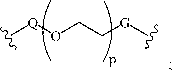

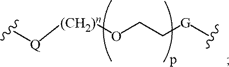

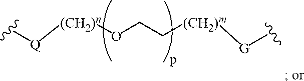

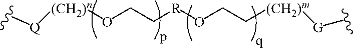

wherein
Q is a bond or a divalent group that forms a bond to X;
R is an alkyl, aryl, heteroalkyl, heteroalkyl, heteroaryl, alkyloxy, alkylamino, alkylglycol, carbonyl, thiocarbonyl, acyl, carbamate, urea, thiocarbamate, thiourea, dithiocarbamate, aminocarbonyl, amide, ester, thioester, thioamide, amine, oxygen, sulfur, sulfone, or sulfoxide, in divalent form;
G is a bond or a divalent group that forms a bond to Y; and
m, n, p, and q, if present, are each independently an integer from 0 to 50 provided that at least one of m, n, p, and q is an integer greater than 0.

In one example, Q and G are, independently, carbonyl, thiocarbonyl, acyl, carbamate, urea, thiocarbamate, thiourea, dithiocarbamate, aminocarbonyl, amide, ester, thioester, thioamide, sulfone, or sulfoxide, in divalent form.

In one example, Q and G are, independently, a carbonyl, or an acyl selected from the group consisting of acetyl, 2-hydroxyacetyl, 2-aminoacetyl, propionyl, 3-hydroxypropanoyl, 3-aminiopropanoyl, butanoyl, 4-hydroxybutanoyl, and 4-aminobutanoyl, each of which is in divalent form.

In one example, m and n, if present, are each independently an integer from 0 to 2, and Q and G are each independently carbonyl, thiocarbonyl, acetyl, 2-hydroxyacetyl, 2-aminoacetyl, propionyl, 3-hydroxypropanoyl, 3-aminiopropanoyl, butanoyl, 4-hydroxybutanoyl, 4-aminobutanoyl, carbamate, urea, thiocarbamate, thiourea, dithiocarbamate, aminocarbonyl, amide, ester, thioester, thioamide, sulfone, or sulfoxide, each of which is in divalent form.

In one example, Z is hydrophilic.

In one example, L has the structure

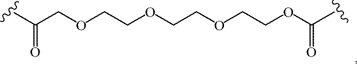

which provides a backbone having a length of 13 atoms as counted in a linear path.

In one example, L has the structure

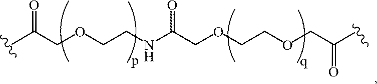

wherein p and q are each independently an integer from 0 to 50.

In one example, L comprises one or more of a divalent alkyl group, a divalent heteroalkyl group, or a divalent polyethylene glycol (PEG), or one or more of each.

In one example, L comprises a 5 or more divalent ethoxy (—CH₂CH₂O—) groups. In one example, L comprises a 1 or more heteroatoms for every 2 carbons and no alkyl chain longer than butyl.

In one example, L attaches to X via an oxygen, nitrogen, sulfur, carbonyl, or ethynyl of X, and L attaches to Y via an oxygen, nitrogen, sulfur, carbonyl, or ethynyl of Y.

In one example, X is an estrogen steroid that includes a divalent group selected from oxygen, amine, sulfur, vinyl, ethyne, and carbonyl, which is positioned at C6, or C17, of the estrogen steroid, and the divalent group at C6 or C17 is attached to L.

In one example, Y is (S,R,S)-AHPC, which is attached to L via an amine of the (S,R,S)-AHPC.

In one example, the compound has the structure:

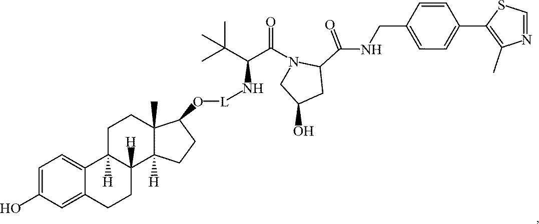

wherein L is the linker.

In one example, the compound has the structure:

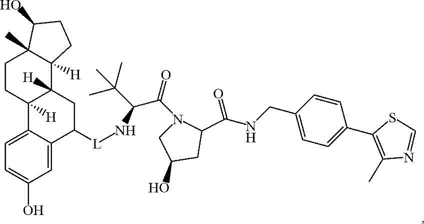

wherein L is the linker.

In one example, the compound has the structure: 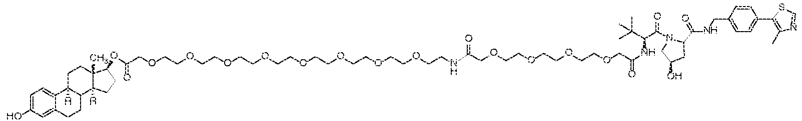 In one example, the compound has the structure:

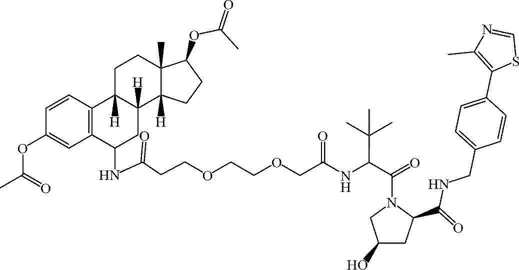

In one example, the compound has the structure:

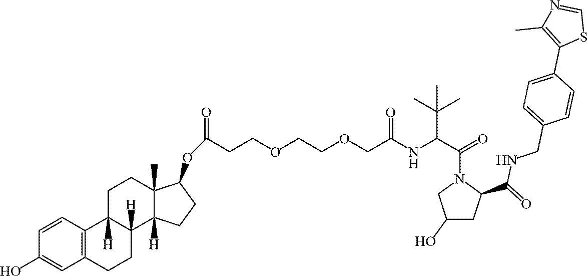 [SMILES: CC(C)(C)OC(=O)COCCOCCOCCOCC(=O)OC(C)(C)C]

In one example, the compound has the structure: 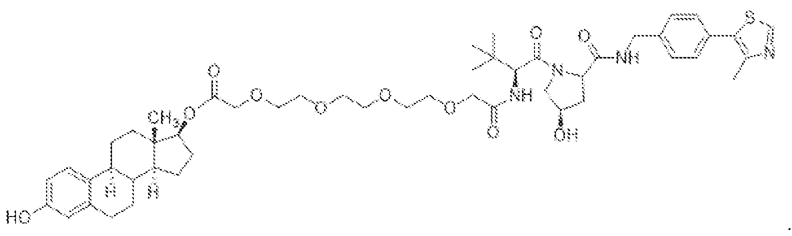 [SMILES: O=C(O)COCCOCCOCCOCC(=O)O] The invention will be described by the following non-limiting examples.

### Example 1

GPER is associated with poor survival and disease progression in breast (Filardo etal., 2000; Filardo et al., 2006; Sjostrom et al., 2014), ovarian (smith et al., 2009) and endometrial (Smith et al., 2007) cancers. Significantly, GPER is expressed in >80% of TNBCs, a breast cancer subtype with poor overall survival due to a lack of expression of the proteins that available drugs target. This estrogen receptor represents an independent measure of estrogen action and is a druggable target. The goal of this project is to take a key step in the development of PROTACs for targeting and degrading GPER

A survey of 121 patients with TNBC suggests that this receptor is expressed in > 80% of these tumors. Expression of GPER in a majority of TNBC is supported by other smaller studies and is in contrast to data from a rather limited study of 6 tumors and 2 cell lines suggesting that GPER is a tumor suppressor in TNBC. Unlike the expression of ER, which is inversely associated with clinical predictors of advanced breast cancer, that of GPER associates directly with these same variables (Filardo et al., 2006; Ignatov et al., 2011), suggesting that it plays a role in metastasis. This observation and the fact that GPCRs are targets of the pharmaceutical industry make GPER a promising druggable target for breast cancer. Thus, therapeutic agents of this type, that selectively degrade GPER, may be useful in the treatment of TNBC, and may also hold utility for other endocrine-resistant breast cancers. Finally, GPER is linked to advanced disease and poor outcome in gynecological cancers, suggesting GPER- PROTACs may benefit these patients.

Estrogen-targeted therapy is effective in postmenopausal women. A spontaneous model of TNBC oncogenesis in which tumor formation occurs postmenopause is used. GPER-PROTAC are delivered to mice with early stage TNBC (tumors ≤ 0.5 cm). Although GPER-PROTACs may function most effectively in the absence of endogenous estrogen, testing of males is also conducted although breast cancer in males accounts for < 1% of all cases (Anderson et al., 2010)

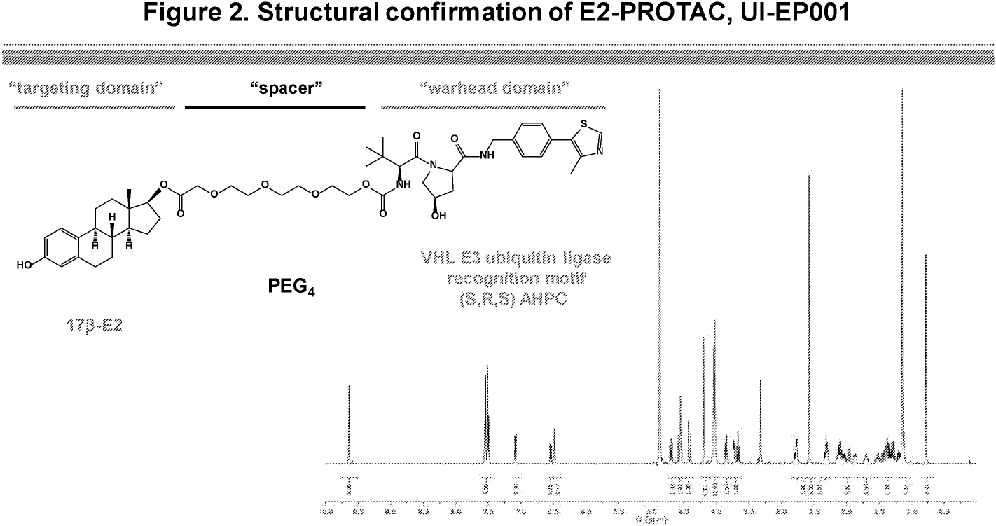

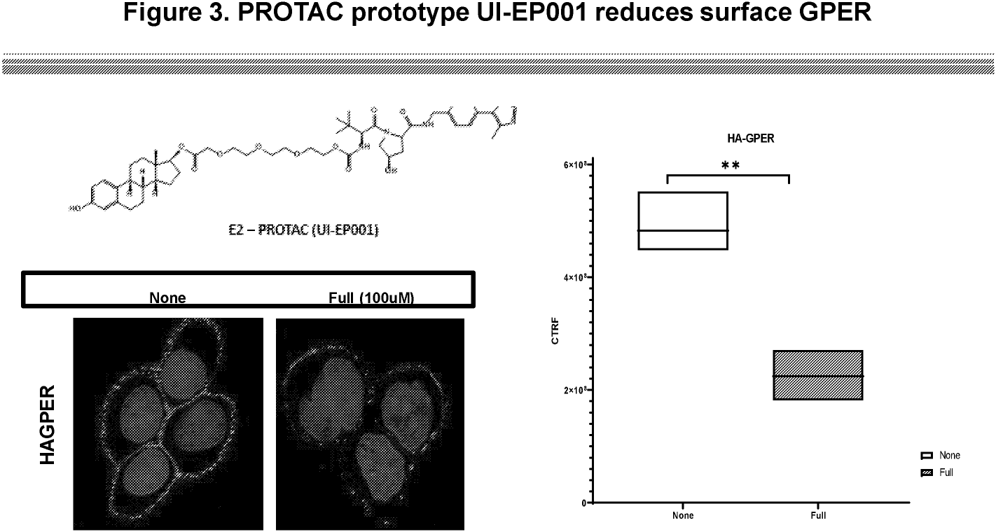

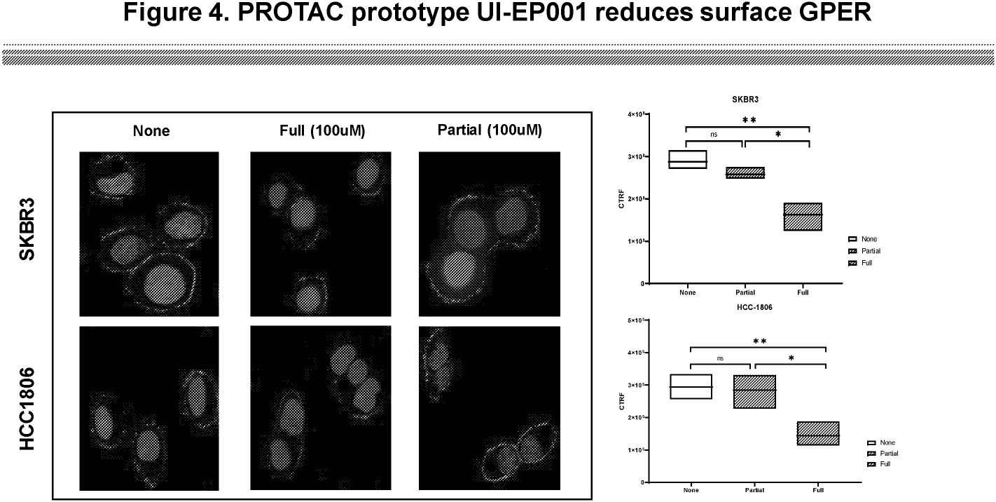

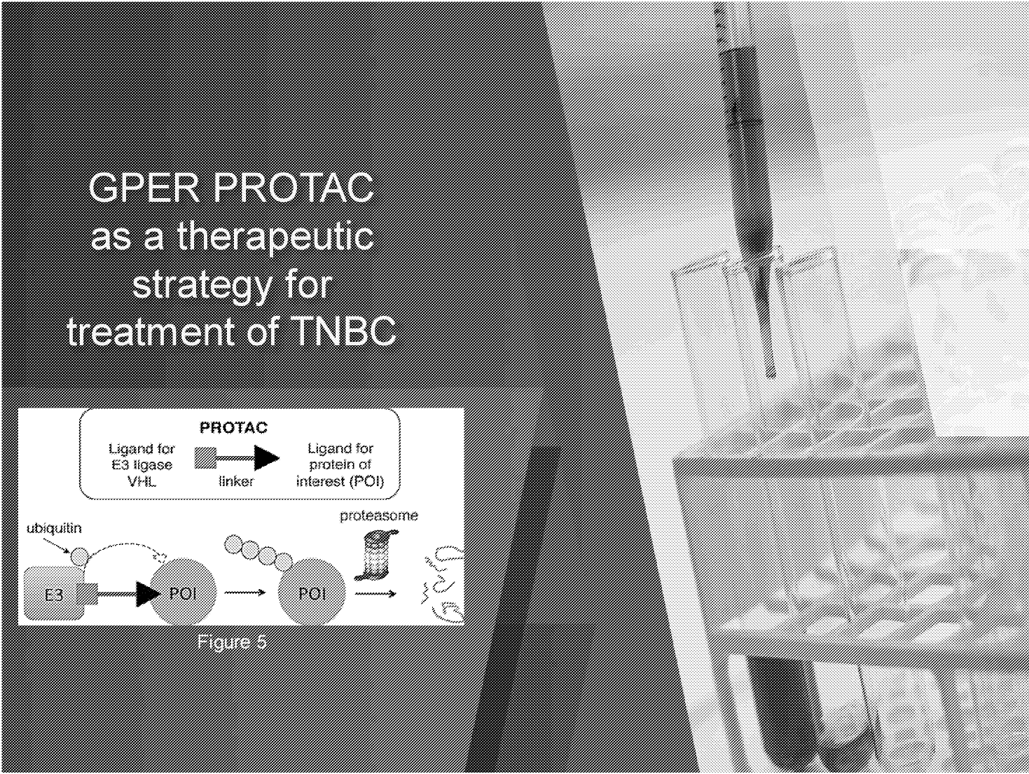

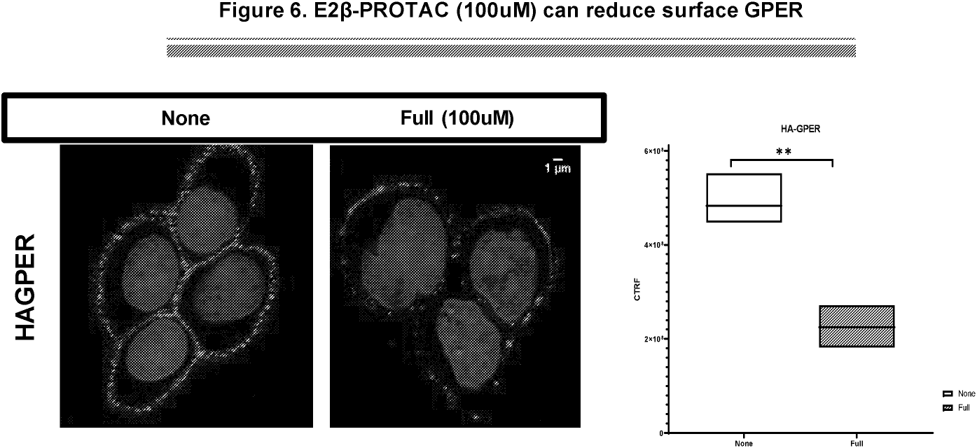

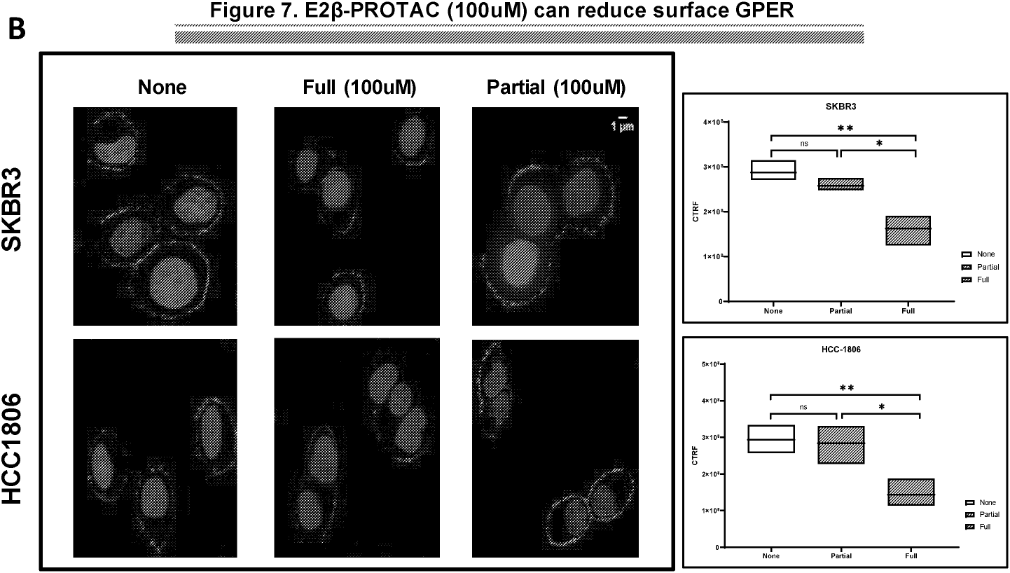

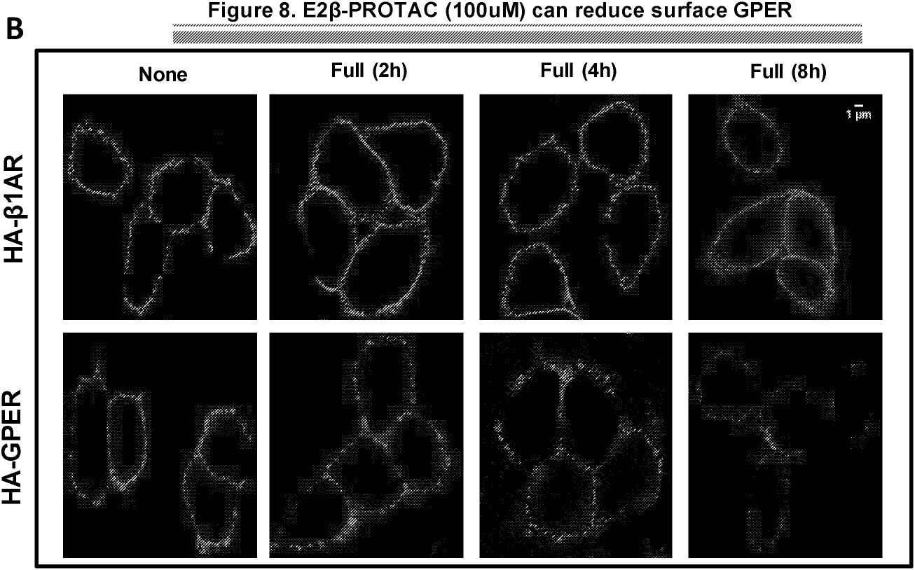

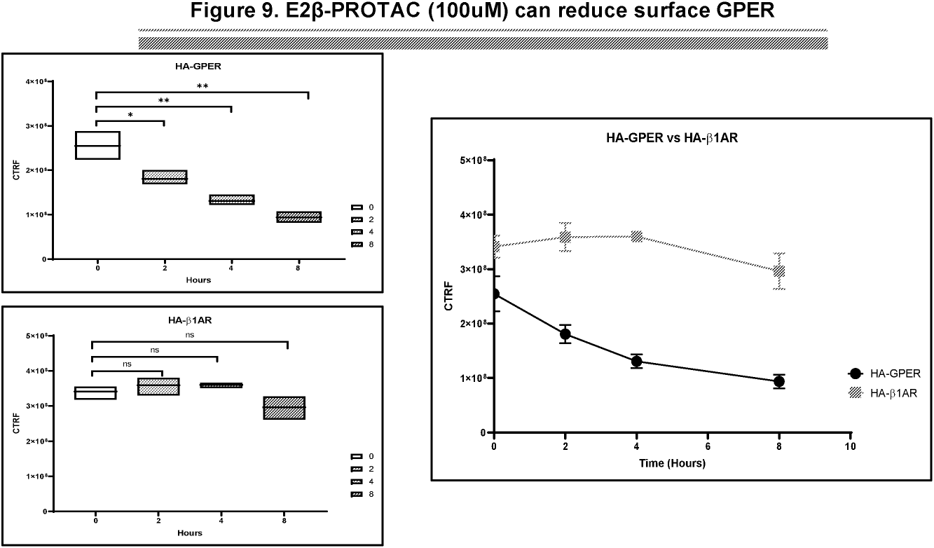

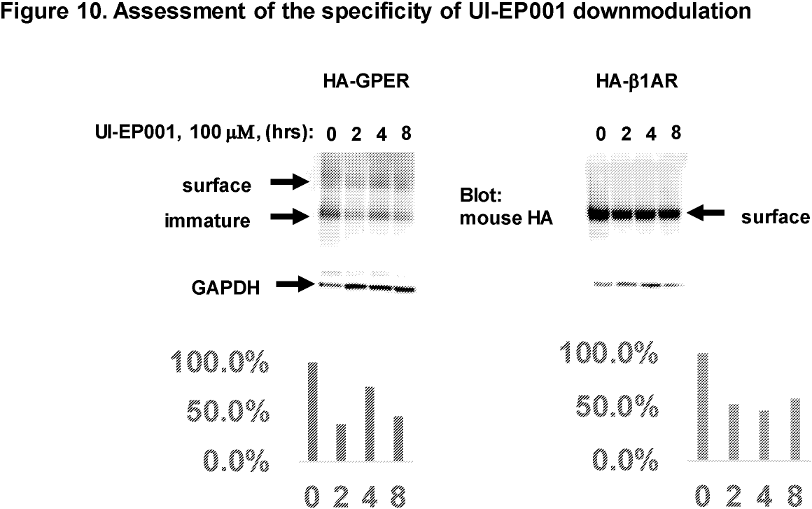

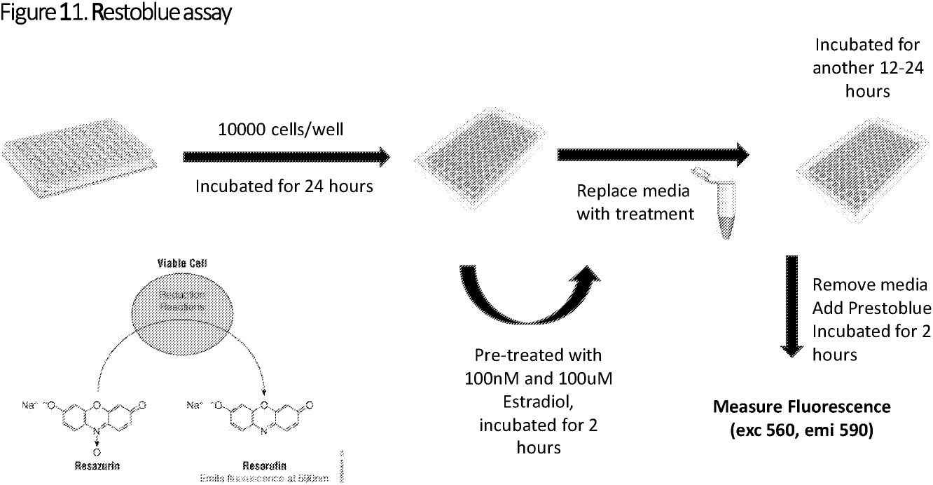

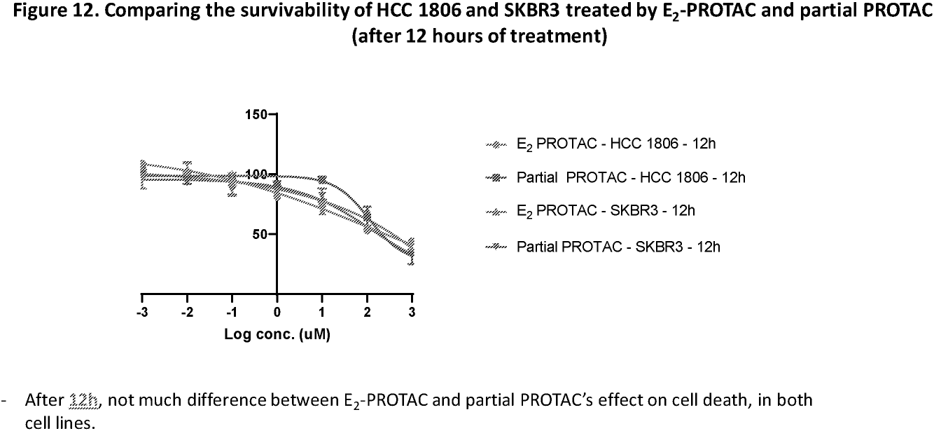

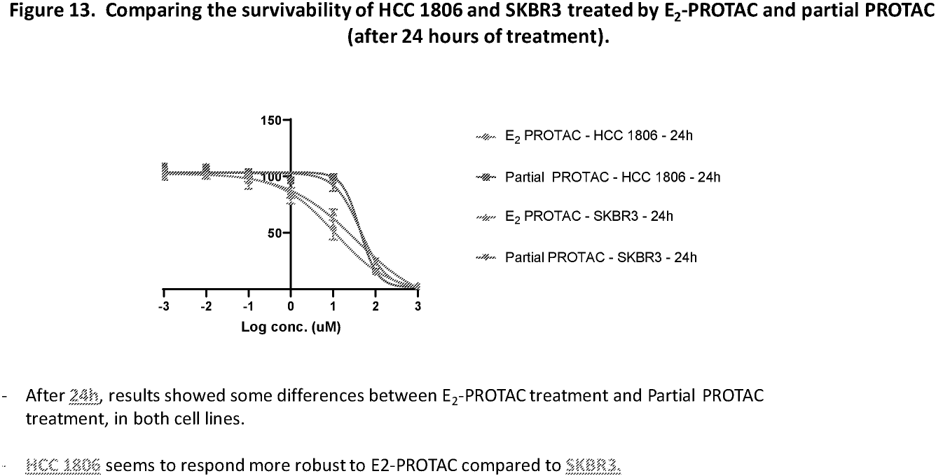

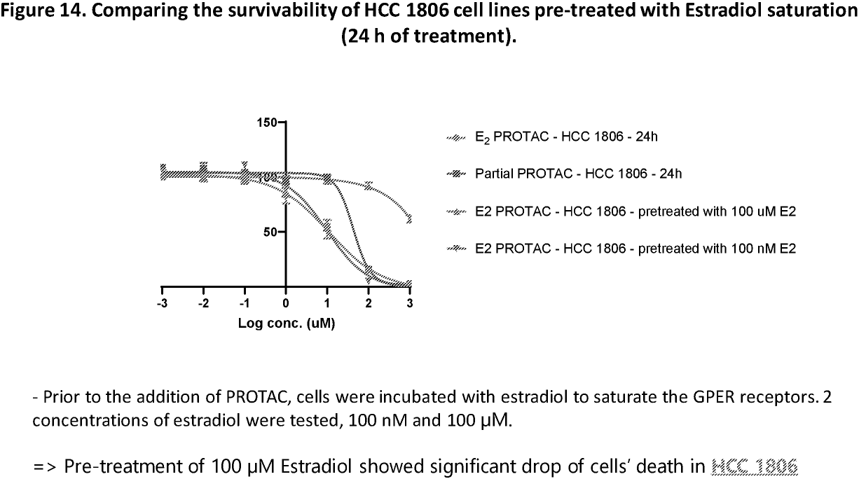

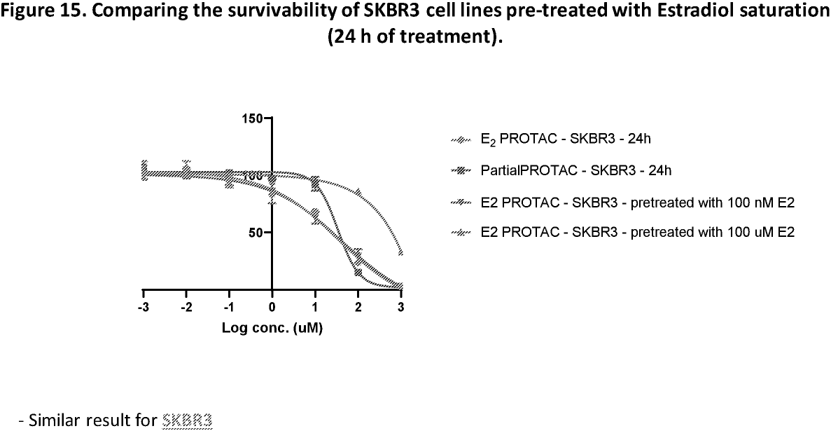

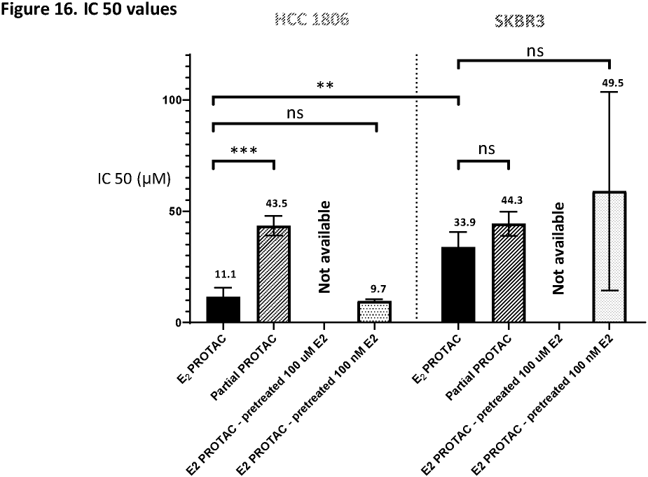

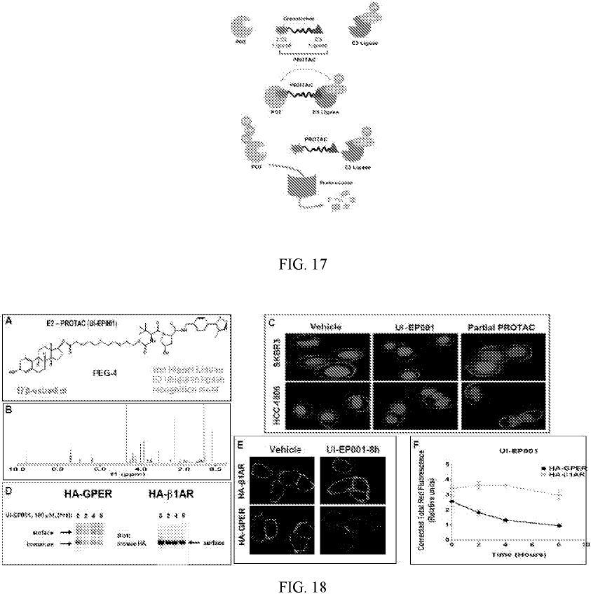

UI-EP001, a GPER-PROTAC prototype, selectively degrades recombinant GPER and downmodulates the expression of native GPER in human breast cancer cells (FIGS. 2 and 3). To screen GPER-PROTACs, a GPER binding assay that measures a high-affinity (Kd= 2.7 nm), limited capacity, displaceable, single binding site specific for estrogens in plasma membranes of human SKBR3 breast cancer cells that express GPER but lack nuclear ER may be used (Thomas et al., 2005). GPER undergoes an uncommon mechanism of endocyosis that requires neither receptor phosphorylation, interaction with β--arrestin nor lysosomal activity. Instead, GPER is polyubiquitinylated at the plasma membrane and trafficks in a retrograde fashion via rab11-positive recycling endosomes to the trans Golgi network (TGN) prior to proteasomal degradation (Cheng et al., 2011). Finally, to quantitate the degree of GPER degradation, a sensitive Nanobit binary luminescence assay for measuring the relative efficacy of GPER-PROTACs to proteolyze GPER is employed (FIG. 11).

Identify GPER-PROTACs with a high binding affinity and specificity for GPER.

Rationale. Proteolytic targeting of ER is an effective means of treating endocrine-resistant breast cancer. ER-PROTACs promote tumor regression in preclinical models of ER+ breast cancer when delivered orally (Flanagan et al., 2019). PROTACs targeting GPER, a distinct estrogen receptor expressed in the majority of ER-negative (Filardo et al., 2006; Ignatov et al., 2011) and triple-negative tumors (97/121= 80.2%;) would complement selective GPER antagonists that have been developed (Prissnitz et al., 2015) but are not yet approved for clinical use, and could potentially be more effective.

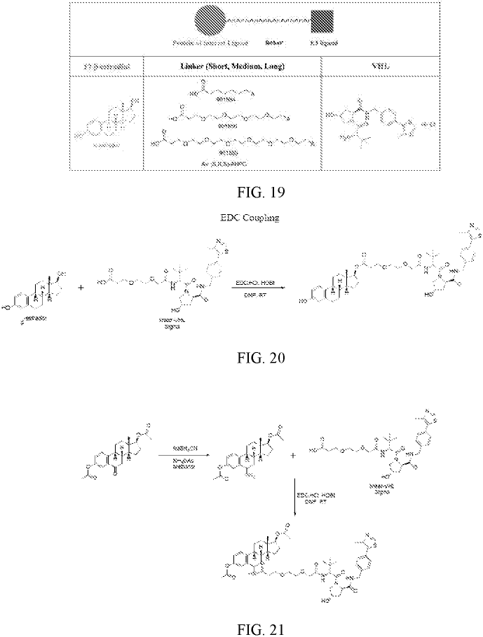

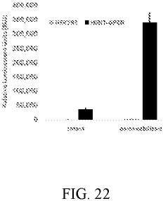

Design of GPER-PROTACs. In one embodiment, PROTAC design is based upon the use of 17 β--E2 as a targeting ligand and the von Hippel-Lindau (VHL)-derived ubiquitin E3 ligase ligand, (2S,4R)-1-((S)-2-Amino-3,3-dimethyl-butanoyl)-4 hydroxy -N-(4-(4-methylthiazol-5-yl) benzyl) pyrrolidine-2-carboxamide, known as (S,R,S) AHPC [24]. GPER-PROTACs are assembled using partial PROTACs consisting of AHPC conjugated to an array of 2, 4, or 6 unit pegylated chemical linkers (FIG. 19). Structure-activity relationship (SAR) data suggests that coupling of the chemical linkers into the C6 and C17 atoms of the estrane ring most likely preserves the estrogen binding function of GPER. Fulvestrant (ICI 182,780), which contains a C6 side chain, has a relative binding activity of 5% and functions as a GPER agonist at 500 nM (Filardo et al., 2007), and the potency of 17 β-E2-hemisuccinate-17-BSA is similar to that of 17 β-E2 with respect to stimulating intracellular cAMP production (Filardo et al., 2007).

Synthesis of GPER-PROTACs_{.} purification and confirmation of structure. C-17 linked GPER-PROTAC is synthesized through EDC coupling of the 17 β-hydroxyl group of 17 β-E2 with (S,R,S)-AHPC-PEG₂-COOH; (S,R,S)-AHPC-PEG₄-COOH or (S,R,S)-AHPC-PEG₆-COOH using EDC.HCI and HOBt in the presence of DMF at room temperature. The synthesis of the desired compounds is confirmed by NMR analysis and high-resolution mass spectrometry. C-6 linked GPER-PROTAC can be synthesized through a multistep reaction. First, reductive amination of 6 carbonyl group of 13-methyl-6-oxo-7,8,9,11,12,13,14,15,16,17-decahydro -6H-cyclopenta[ajpheiianthrene-3,17-diyl diacetate (1) using sodium cyanoborohydride in the presence of ammonium acetate in refluxing methanol. Second, coupling of 6-amino-13-methyl-7,8,9,11,12,13,14,15,16,17-decahydro-6H-cyclopenta[a]phenanthrene-3,17-diyl diacetate (2) with (S,R,S)-AHPC-PEG₂-COOH; (S,R,S)-AHPC-PEG₄-COOH or (S,R,S)-AHPC-PEG₆-COOH using EDC.HCI and HOBt in the presence of DMF. Purification of compounds is achieved by C18 reverse phase preparative HPLC columns. The synthesis and purity of the desired compounds is confirmed using NMR analysis and LC-MS. All of the chemicals to be used in this synthesis are commercially available.

Evaluation of GPER-PROTAC binding. GPER-PROTACs are tested for binding activity using a competitive radioreceptor assay [22]. In brief, GPER-PROTACs are dissolved in ethanol and added to glass reaction tubes for a final concentration of 1 nM to 10 uM; the ethanol is evaporated under nitrogen. Membrane proteins (100 ug) and 4 nM (89 Ci/mmol) ³H-17 β-estradiol (E2) is added to the tubes and incubated at 4° C. for 30 min. Binding is terminated by rapid filtration over presoaked glass fiber filters to separate bound from free ³H-E2. Filters will be washed and radiation is measured using a liquid scintillation counter. Maximum specific binding is calculated as the difference between total binding (absence of competitor) and nonspecific binding in the presence 1,000-fold molar excess of either 17 β-E2 or GPER-PROTAC. The concentration of competitor needed to displace 50% maximum specific binding (EC₅₀) is calculated from one-site competitive binding curves, where the top and bottom of the curves are defined as 100% and 0%, respectively. Relative binding affinity (RBA) is calculated as the ratio of the EC₅₀ of 17 β-E2 to the EC₅₀) of GPER-PROTAC, expressed as a percentage. In one embodiment, the RBA of one or more GPER-PROTAC for 17β-E2 is >10%.

Test GPER-PROTAC candidates for their capacity to degrade GPER and reduce its signaling activity. Rationale. This addresses the capacity of the GPER-PROTACs to recruit E3 ubiquitin ligase and ubiquitinylate GPER, resulting in its destruction by the proteasome. The degradatory capacity of GPER-PROTACs is evaluated first by quantitative immunofluorescent and immunochemical analyses. Drug candidates showing strong degradatory capacity then is assessed further using a high throughput, sensitive luminescent assay. Finally, the efficacy of each tested GPER-PROTAC in reducing active GPER is assessed by measuring GPER-dependent erbB1 tyrosyl phosphorylation and phosphorylation of erk-½ (Prissnitz et al., 2015). Collectively, these measures of GPER downmodulation identify GPER-PROTACs that effectively reduce GPER activity.

In breast cancer cells, the bulk of GPER is in its low affinity state, uncoupled from its heterotrimeric G proteins and retained in the endoplasmic reticulum. Only a small fraction is expressed at the plasma membrane in its high affinity, G-protein coupled state (Prissnitz et al., 2015; Smith et al., 2007). When used at high concentrations in intact cells, GPER-PROTACs may degrade both low and high affinity receptors. The impact of GPER-PROTACs on cell surface-associated and total GPER is measured. As mentioned above, and detailed below, several quantitative modalities are used to measure GPER degradation and its signaling activity in breast cancer cells that have been treated with GPER-PROTAC candidates, e.g., in breast cancer cells of luminal A, luminal B, her2 overexpressing and basal immunophenotypes that have been treated with GPER-PROTAC candidates. Finally, the relative efficacy of the existing GPER antagonists, G15 or G36, in reducing GPER signaling activity, is compared. Evaluation of GPER degradation. GPER-PROTACs with the highest RBA are tested for their ability to ubiquitinylate and degrade GPER. Breast cancer cells expressing GPER are treated with various doses of GPER-PROTAC or vehicle for 5 min to 8 hrs and GPER downmodulation is assessed by both immunofluorescent and immunochemical analyses. The presence of native GPER is detected in fixed intact or detergent-permeabilized breast cancer cells using either N-terminal or C-terminal antibodies. GPER detected by immunofluorescence is quantified by total corrected cell fluorescence (J-Image) and by ELISA. Steady state levels of the immature or mature forms of GPER protein are determined by immunoblotting with GPER specific antibodies. In these experiments, the degree of GPER degradation is determined by measuring band pixel intensities (image J) and normalizing to GAPDH. The specificity of GPER action is confirmed in the presence of an excess of free 17 β-E2 or control steroid (17 β-estradiol, which shows no measurable binding to GPER) (Thomas et al., 2005). Partial PROTACs lacking 17 β-E2 or coupled to 17 β-E2 serve as drug controls. Targeting specificity is evaluated in HEK293 cells stably expressing HA-GPER, HA-CXCR4, or HA- β-LAR (Filardo et al., 2018).

Evaluation of GPER polyubiquitinylation. Ubiquitinylation status is confirmed in Tandem Ubiquitin Binding Element (TUBE) pull down assays, blotting with GPER-specific antibodies. Again, the degree of GPER ubiquitinylation in both its immature and surface form ia quantified by measuring pixel band intensities in lysates from untreated, PROTAC-treated and partial PROTAC-treated cells. Proteasomal degradation is assessed by treating cells with the proteasome inhibitor, MG132, while control cells receive chloroquine which inhibit lysosomal hydrolases. A lysineless versions of GPER is also tested (Lys333Arg, Lys341Arg, Lys351Arg) which can not be ubiquitinylated for their sensitivity to GPER-PROTAC degradation.

Nanobit complementation assay to assess degradation of surface and total GPER. Nanobit™ (Promega) binary luminescence complementation assays are performed to quantitate the removal of GPER from the cell surface as well as its total degradation. This assay records a luminescent signal that has a dynamic range over 6 logs. A luminescent signal is generated in the presence of luciferin substrate upon the interaction of two split components of Sea Shrimp luciferase, termed HiBit and LgBit. For these experiments, soluble LgBit is delivered with luciferin to intact or detergent-permeabilized cells expressing GPER with an N-terminal HiBit tag.

Evaluation of GPER signaling. Following treatment for various lengths of time with GPER-PROTACs, cells are stimulated with 17 β-E2 and GPER activity is measured by monitoring erbB1 tyrosyl phosphorylation or erk-½ phosphorylation. Measurement of EGF-stimulated erbB1 or erk-½ serves as a control for the specificity of GPER-PROTACs. Additional control experiments measuring erbB1 or erk-½ stimulation are conducted with partial PROTACS coupled to 17 β-E2. Data are quantitated by pixel band intensities and normalized to untreated cells

Determine the in vivo efficacy of lead GPER-PROTACs.

Rationale. Early consideration of efficacy, biodistribution, and toxicity is important in the initial assessment of a rationally-designed drug and in subsequent modification of its structure. These biological responses are measured following oral administration of GPER-PROTACs.

GPER acts in cancer cells, promoting their survival, and it also affects cells that define the tumor microenvironment and promote cancer progression (Filardo et al., 2018). Thus, the anti-oncogenic or antitumor activity of GPER-PROTACs is determined using a mouse model that simulates human TNBC oncogenesis, e.g., by measuring inhibition of tumor growth or animal survival. Toxicity and biodistribution studies are conducted in both wild-type and GPER-null mice. Experiments are performed in ovary-intact and ovariectomized (OVX) mice.

Statistical analysis. Twelve (12) mice per group achieves 80% power to detect a mean difference of 1.2 times group-specific standard deviations. Power is conservatively estimated based on using a two-sided, two-sample t-test at one time point with a significance level of 5%. Mixed effects regression will be used to estimate and compare tumor growth rates as a function of time and treatment group. A random effects model will be utilized to account for the longitudinally correlated nature of repeated tumor measurements. This formal analysis is expected to have higher power since all tumor measurements across time will be utilized. For overall survival, curves for each treatment group will be constructed using the Kaplan-Meier method, and they will be compared using the log-rank test.

GPER-PROTACs as cancer drugs_{.} GPER-PROTACs found to have the highest in vitro efficacy are examined for their anti-oncogenic efficacy. Male p53^{fl/fl}Brca1^{fl/fl} mice will be bred with female K14-cre; p53^{fl/fl}Brca1^{fl/fl} mice, which results in normal litter sizes and demonstrates that the resulting transgenic pups due not suffer from embryonic lethality. Approximately 85% of female mice develop mammary tumors with a median survival time of 248 days. Tumor formation is monitored carefully using digital calipers, and when tumors reach 5 mm in one dimension, mice are randomized into control and treatment groups. Initial experiments focus on identifying a dose and treatment schedule for which an impact on tumor growth (regression) is observed, with GPER-PROTAC dosing (100-4,000 mg/kg) guided by toxicity tests. Mice receive a single dose of vehicle or GPER-PROTAC by gavage. Mice are monitored for tumor growth using calipers. Tumor growth and overall survival (OS) are used as measures of responsiveness to therapy.

Biodistribution studies. GPER-PROTACs are administered to wild-type and GPER-null mice by gavage. The dose schedule and dose concentration may vary. Tumors and tissues from the breast, ileum, duodenum, spleen, heart, lungs and liver are extracted, and drug concentration are measured by LC-MS.

Tumors and tissues are harvested at 1 hr, 4 hrs, 24 hrs, 7 days, 14 days, 21 days and 28 days post inoculation.

Toxicity studies. To identify any toxicities associated with therapy using GPER-PROTACs, liver function (ALT, AST, LDH, bilirubin), kidney function (creatinine, BUN), and inflammatory bowel disease (IBD) (diarrhea, bloody stools) are monitored at weekly intervals, in both treated and untreated mice (5 per group), in conjunction with the experiments outlined above. Objective criteria indicative of toxicity include a 3-fold or greater elevation of liver and kidney function values, and unabated diarrhea and/or bloody stools.

As depicted in FIG. 11, after 24 hours of treatment, HCC 1806 and SKBR3 cells showed lower survivability when treated with E₂-PROTAC compared to partial PROTAC. HCC 1806 showed higher response to E2-PROTAC than SKBR3. The IC 50 ranged from about 10 to about 50 uM. Pre-treatment of cells with estradiol showed a significant drop in cell death, in both HCC 1806 and SKBR3. The degradation of GPER may result in cell death or a decrease in cell proliferation. Or the delocalization of GPER from the membrane to the nucleus may result in recruitment of a ligase to degrade protein inside cytoplasm/nucleus.

### Example 2

Decisions regarding the assignment of hormonal therapy for breast cancer are based solely upon the presence of nuclear estrogen receptors (ERs) in biopsied tumor tissue. This is despite the fact that the G-protein-coupled estrogen receptor (GPER) is linked to advanced breast cancer and is required for breast cancer stem cell survival; an observation which suggests that effective endocrine therapy should also target this receptor. Here, two ER/GPER-targeting proteolytic chimeras (UI-EP001 and UI-EP002) are described that effectively degrade ERa, ERb and GPER. These chimeras form high affinity interactions with GPER and ER with binding dissociation constants of about 30 nM and 10-20 nM, respectively. Plasma membrane and intracellular GPERs and nuclear ERs were degraded by UI-EP001 and UI-EP002, but not by a partial PROTAC lacking its estrogen-targeting domain. Pretreatment of cells with the proteasomal inhibitor, MG132, blocked UI-EP001 and UI-EP002 proteolysis, while the lysosomotrophic inhibitor, chloroquine, had no effect. Off-target activity was not observed against recombinant b1-adrenergic receptor or CXCR4. Target specificity was further demonstrated in human MCF-7 cells where both drugs effectively degraded ERa, ERβ, and GPER yet spared the progesterone receptor (PR). UI-EP001 and UI-EP002 induced cytotoxicity and G2/M cell cycle arrest in MCF-7 breast cancer and human SKBR3 (ERa-ERb-GPER+) breast cancer cells but not human MDA-MB-231 breast cancer cells which do not express functional GPERs/ERs. These results provide for a receptor-based strategy of anti-estrogen treatment for breast cancer that targets both plasma membrane and intracellular estrogen receptors.

### Materials and Methods

Molecular docking of E2-PROTAC to ERa or GPER. Previously established GPER homology models were used in the GLIDE (Schrödinger, Cambridge, MA) docking studies (Amatt et al., 2012; Amatt et al., 2013). In these studies, a grid was first built around the previously established binding site of E2 using Receptor Grid Generation within GLIDE. UI-EP001 and Ul-EP002 were drawn into Maestro 11.3 (Schrödinger, Cambridge, MA) and were prepared for docking using the LigPrep function within Schrödinger. The OPLS3 force field was used and possible ionization states at pH 7 ± 2 were generated using Epik; all other settings were kept at their default settings. GLIDE docking of UI-EP001 and Ul-EP002 was then performed. Van der Waals radii were set to a scaling factor of 0.8 with a partial charge cutoff of 0.15. XP (extra precision) docking was performed with flexible ligand sampling with both sample nitrogen inversions and sample ring conformations turned on. Bias sampling for amides was set to penalize nonplanar conformations. Epik state penalties were added to the docking scores. For docking, 10,000 poses per ligand were kept for the initial phase of docking and the best 1,000 poses per ligand were kept for energy minimization using the OPLS3 force field. Post-docking minimization was performed and 100 poses per ligand were kept. All other settings were kept at their default settings. Both UI-EP001 and UI-EP002 were also docked within ERα in an analogous method. An ERα homodimer crystal structure was used (PDB: 1A52, Tanenbaum et al., 1998) and prepared for docking by removing the bound E2 molecules and then using Receptor Grid Generation within GLIDE before docking studies with UI-EP001 and UI-EP002.

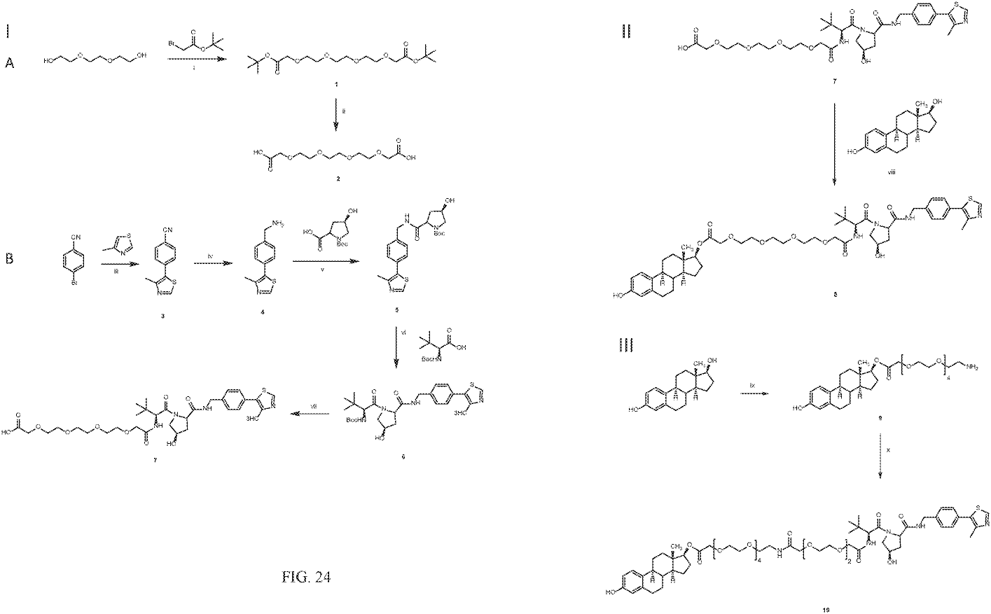

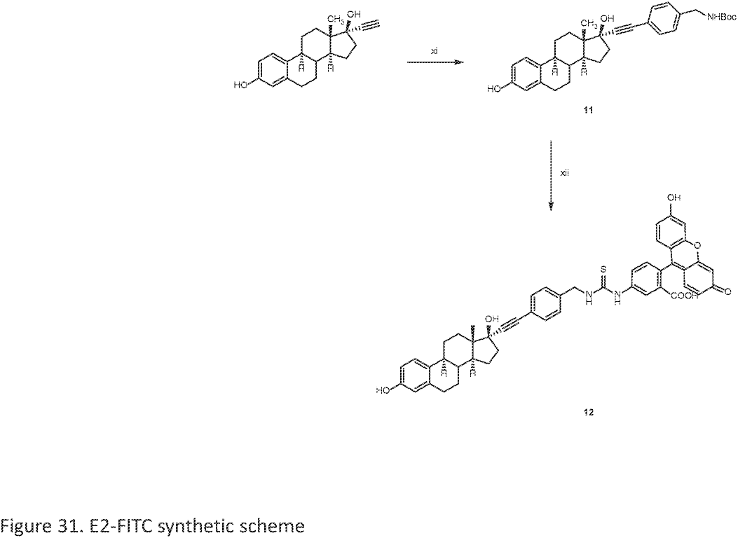

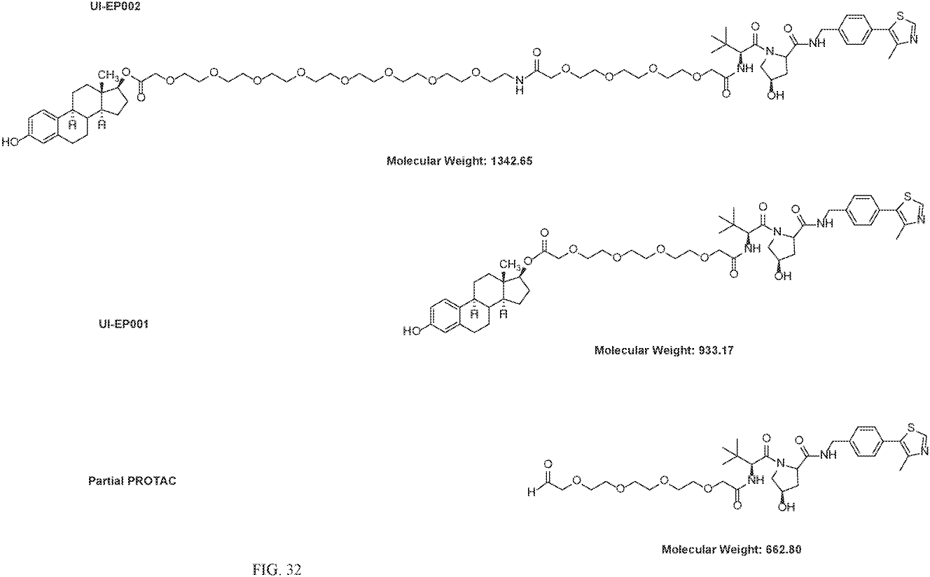

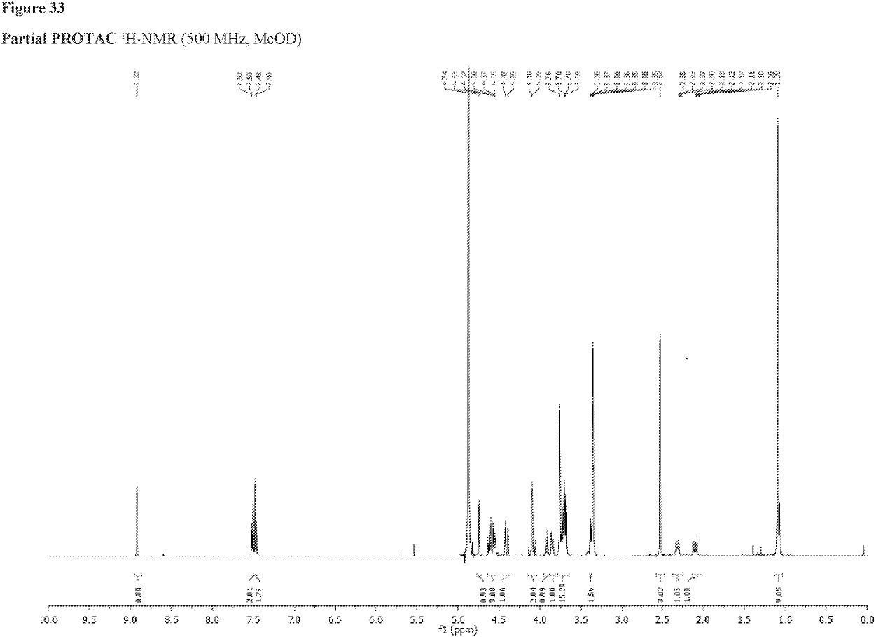

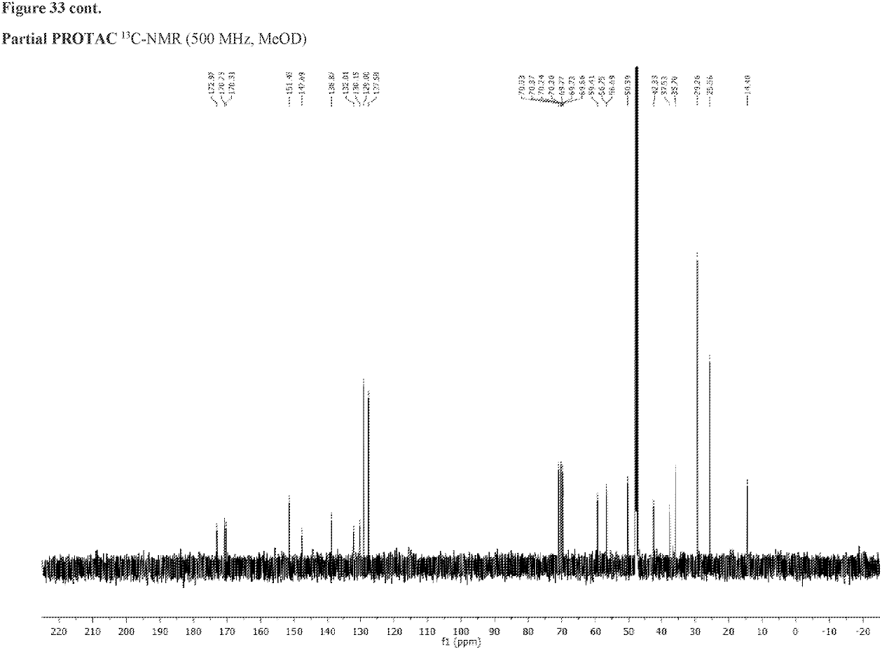

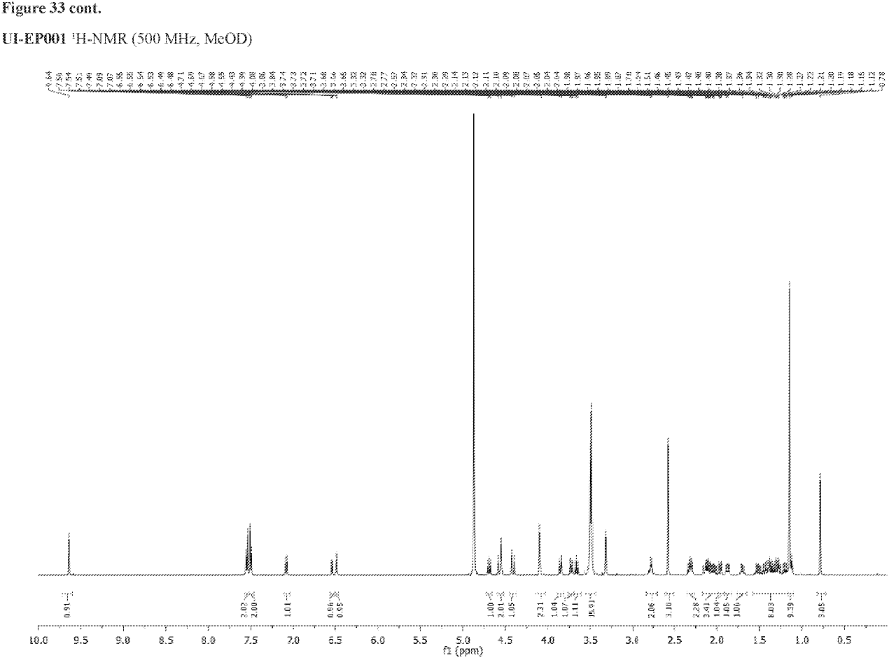

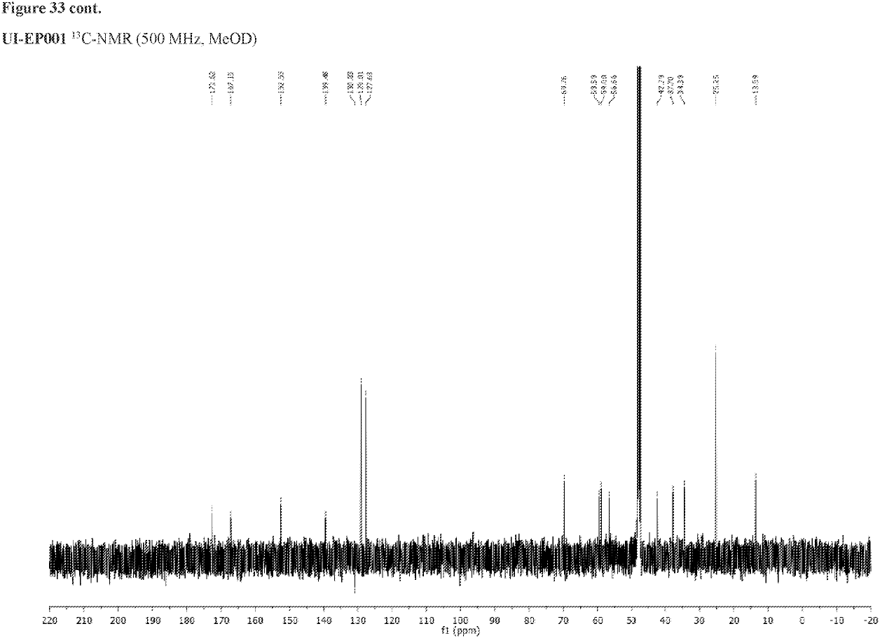

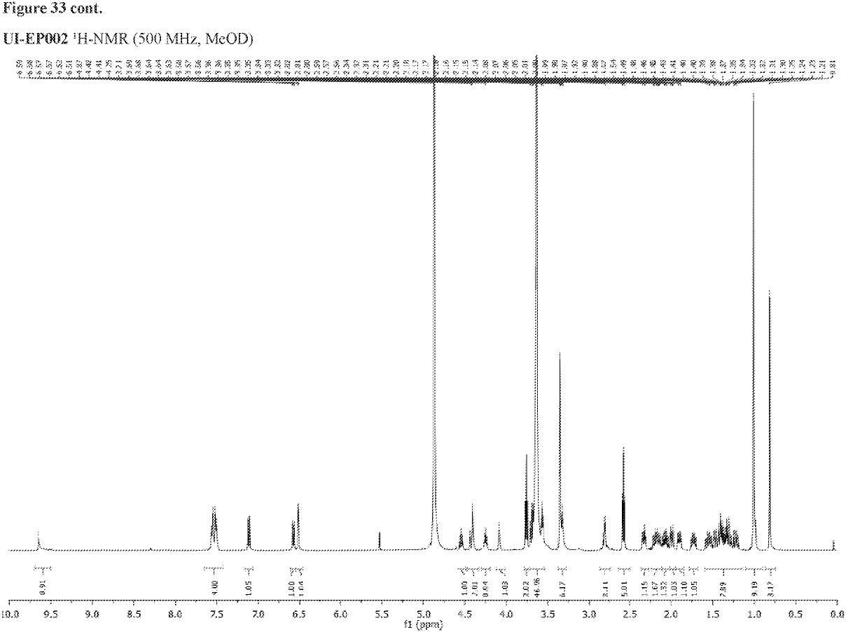

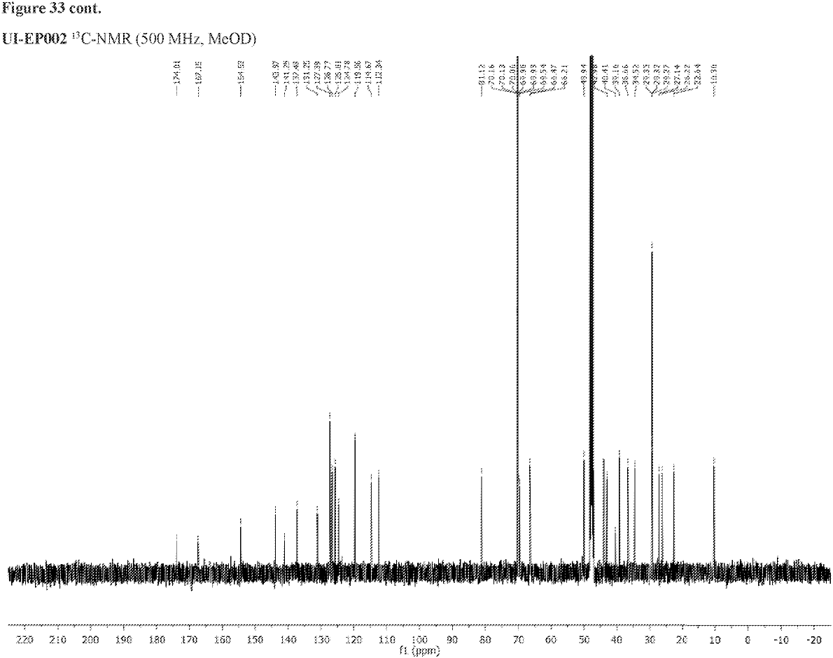

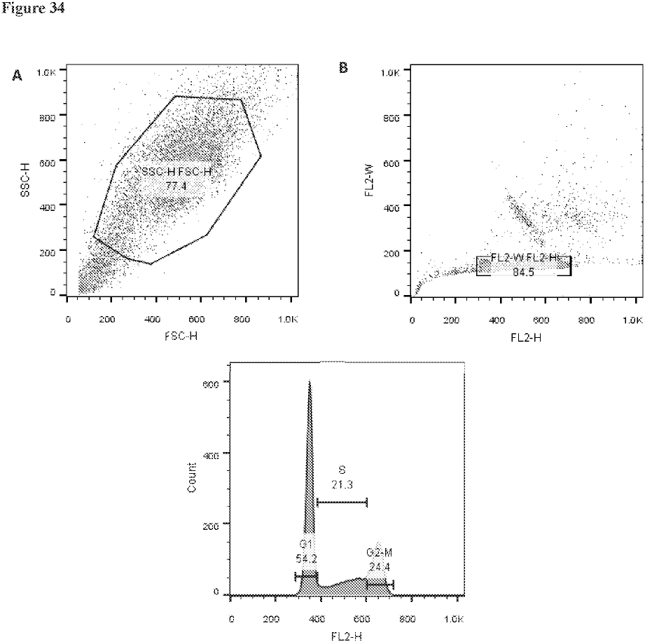

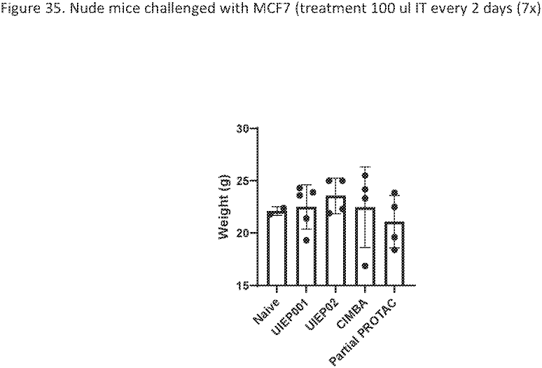

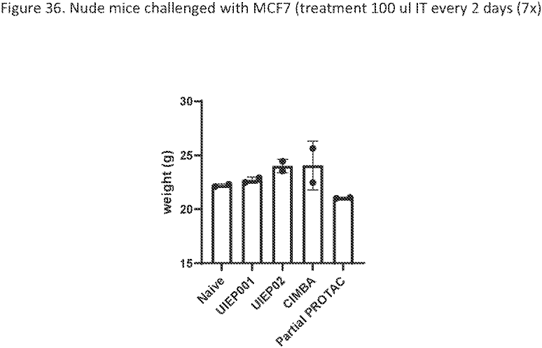

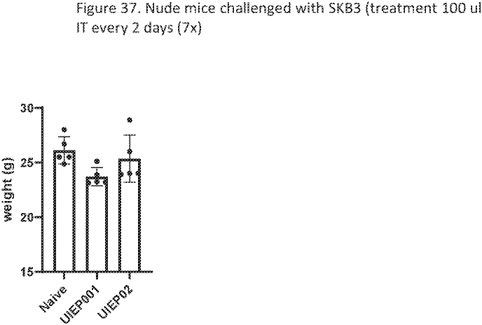

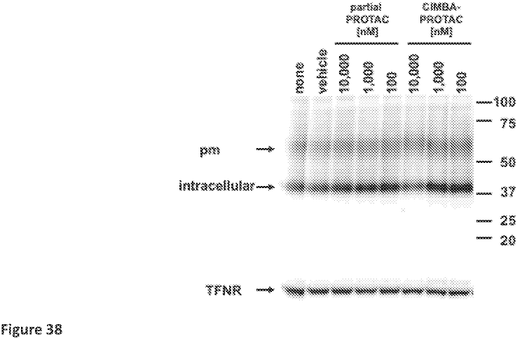

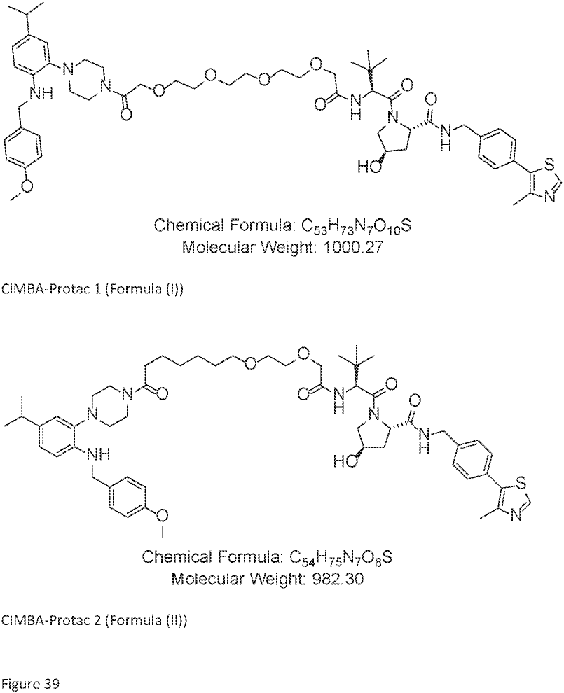

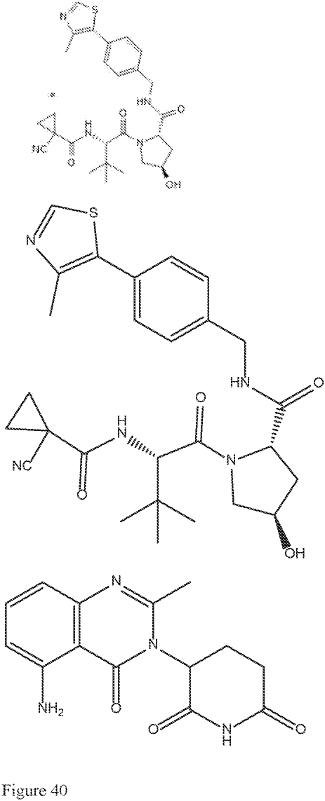

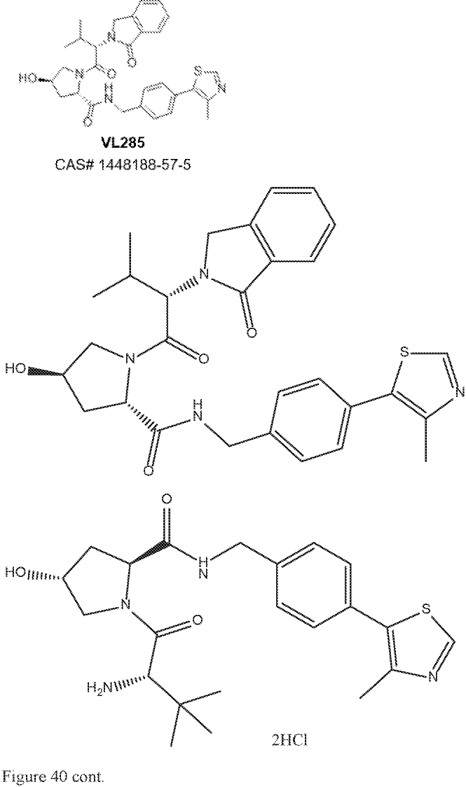

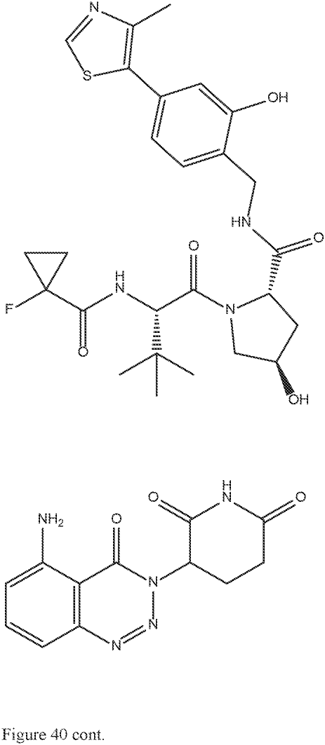

Synthesis of E2-PROTACs and E2-FITC. Chemical synthesis of UI-EP001, UI-EP002, and E2-FITC was conducted according to the schematics in FIG. 24 and FIG. 31, respectively. ¹H and ¹³C NMR spectra were recorded on a Bruker AVANCE AV-300 and Bruker AVANCE AV-500 instrument at 300 K. 1H NMR spectra are reported in parts per million (ppm) downfield from tetramethylsilane (TMS). All 13CNMR spectra are reported in ppm and obtained with 1H decoupling. In the spectral data reported, the format (δ) chemical shift (multiplicity, J values in Hz, integration) was used with the following abbreviations: s = singlet, d = doublet, t = triplet, q = quartet, m = multiplet. MS analyses were carried out with Waters Q-Tof Premier mass spectrometer. UI-EP001 and Ul-EP002 were purified by C18 reverse-phase preparative HPLC column with a Shimadzu Nexera X2 UHPLC System, with solvent A (0.1 % TFA in H₂O) and solvent B (0.1 % TFA in MeCN) as eluents.

Cells and culture conditions. Human MCF-7, MDA-MB-231, HCC-1806, and SKBR3 breast carcinoma cells and human embryonic kidney 293 cells (HEK-293) were purchased from the American Tissue Culture Collection (Manassas, VA). HEK-293 cells that stably express hemagglutinin-tagged GPER (HA-GPER), β1-adrenergic receptor (HA-β1AR), and CXCR4 (HA-CXCR4) have been previously described (Filardo et al., 2007) All cell lines were cultured at 37° C. in a humidified chamber containing 5% CO₂ and in phenol red-free (1:1) DMEM/Ham’s F-12 medium (PRF-DF12) (Invitrogen, Carlsbad, CA) supplemented with 10% fetal bovine serum and penicillin-streptomycin.

Steroids and inhibitors. 17β-estradiol (17b-E2) and aldosterone were purchased from Sigma-Aldrich (St. Louis, MO). The 26S-proteasome inhibitor, MG132 was purchased from Selleckcheryi (Pittsburgh, PA) and the lysosomotrophic agent, chloroquine diphosphate was purchased from Bio-Techne (Minneapolis, MN).

Antibodies. GPER-specific antibodies were generated in New Zealand White Rabbits against a synthetic peptide derived from amino acids 1-62 from the N-terminus of the human GPER polypeptide (Pacific Immunology, Rartiona, CA). Commercial antibodies included: rabbit anti-HA epitope antibody (Cell Signaling Technologies, Beverly, MA), mouse monoclonal ER-α (2Q418), PR (F-4) were purchased from Santa Cruz Biotechnology (Santa Cruz, CA), and rabbit monoclonal ER-β antibody, clone 68-4 was purchased from EMD Millipore (Billerica, MA). Goat anti-rabbit Alexa-Fluor 594, goat anti-rabbit Alexa-Fluor 488 and goat anti-mouse Alexa-Fluor 488 secondary antibodies were purchased from Abeam (Cambridge, MA), Goat anti-rabbit IgG and goat-anti-mouse horseradish peroxidase (HRP)-conjugated antibodies were purchased from Southern Biotechnology (Birmingham, AL).

Plasmids. Molecular clones encoding full-length human GPER (Filardo et al., 2007) and Era (deConink et al., 1995) under the transcriptional control of the CMV promoter have been described. The HiBiT tag (VSGWRLFKKIS) was inserted in-frame at the amino terminus of GPER by inverse PCR using forward 5′-GTTCAAGAAGATTAGCGATGTGACTTCCCAAGCC-3′ (SEQ ID NO:1) and reverse 5′-AGCCGCCAGCCGCTCACCA-TGTCTCTGCACCGTGC-3′ (SEQ ID NO:2) oligonucleotide primers and the Q5 mutagenesis kit (New England Biolabs, Salem, MA). A similar inverse PCR strategy was employed to insert the HiBiT tag at the carboxyl terminus of ER-α using forward 5′-TGGCGGCTGTTCAAGAAGATTAGCTGAGAGCTCCCTGGCGGA-3′ (SEQ ID NO:3) and reverse 5′-GCCGCTCACAG-AGCCTCCTCCACCGACTGTGGCAGGGAAACCC-3′ (SEQ ID NO:4) oligonucleotide primers.

Transient transfections and binary Nano-Bit ™ luminescence assays. Total HiBiT-tagged GPER or ER estrogen receptors were detected in detergent-permeabilized cells by binary luminescence complementation with recombinant LgBit and substrate using the Nano-Glo HiBiT Extracellular Detection system kit (Promega Corporation, Madison, Wl). In brief, HEK293 cells (0.75 × 10⁶) were seeded in 35 mm tissue culture dishes and 24 hours later were transiently transfected with 50 ng of HiBiT-GPER or HiBiT-ERa and 950 ng of pcDNA3.1(+) zeo carrier plasmid using Lipofectamine 2000 (Invitrogen, Carlsbad, CA). The next day transfected cells were harvested by trypsinization and seeded at a density of 10⁴ cells/well in 96-well poly-L-lysine-coated Greiner white-bottomed microplates. On the following day, cell culture media was aspirated and replaced with 100 µL of serum-free PRF-DF12 containing E2-PROTAC, partial-PROTAC, or vehicle at various concentrations and for indicated time intervals at 37° C. In some experiments, chloroquine (100 mM) or the proteasomal inhibitor, MG132 (10 mM) were included. Following treatment, total HiBiT-tagged receptor was measured in cells which were permeabilized in 0.05% Triton X-100 using HiBiT complementation reagent consisting of LgBiT protein and Nano-Gio® HiBiT substrate. Luminescence was measured using the Infinite 200 PRO multimode microplate reader from Tecan (Raleigh, NC) and reported as Relative Luminescent Units (RLU). All samples were measured in triplicate and expressed as the mean value plus or minus standard deviation.

Immunocytofluorescence. Cells were seeded onto poly-L-lysine-coated, 18 mm glass coverslips at a density of 12,500 to 25,000 cells/cm² in PRF-DF12 containing 5% FBS in 12-well cluster plates (CoStar, Corning, NY). The next day, cells were left untreated or treated at 37° C. for 1 hour with 100 mM of Ul-EP001, UI-EP002, or partial PROTAC in the presence or absence of chloroquine (100 mM) or the proteasomal inhibitor, MG132 (10 mM). Following treatment, plates were chilled on ice for 10 minutes and then labeled with GPER N-terminal peptide antibodies for 30 minutes at 4° C. Excess antibody was removed by washing with cold PBS and cells were then fixed in 4% paraformaldehyde in PBS for 5 minutes. Cells were then washed twice in PBS and nonspecific antibody binding sites were blocked in PBS containing 5% bovine serum albumin (BSA) and 5% normal goat serum for 1 hour. Total receptor was measured in cells that were permeabilized in 0.1% Triton X-100 for 10 minutes prior to immunostaining. Fixed, permeabilized cells were washed twice in PBS and incubated in primary antibodies for 1 hour. Excess primary antibody was removed by washing in PBS and cells were then exposed to goat anti-rabbit or anti-mouse secondary antibodies for an additional hour. Following this second incubation, cells were washed once with PBS, once with tris-buffered saline prior, and then coverslips were mounted in Vecta-Shield anti-quench media containing DAPI (Vector Laboratories, Burlingame, CA). Immunofluorescence images were visualized with an Eclipse 80i microscope (Nikon, Inc., Melville, NY) equipped with a Nikon Plan Fluor 100 × 0.5-1.3 oil iris with differential interference contrast and epifluorescence capabilities using a Qimaging Retiga 2000R digital camera and Nikon imaging software (NIS-Elements-BR 3.0), Post-capture, images were processed with brightness/contrast adjustment using Photoshop CS2 (Adobe).

Competitive binding assays. GPER binding was measured in an intact cell-based competitive binding assay using fluorescein-labeled estradiol (E2-FITC) as tracer as described with minor modifications (Cao et al., 2017). SKBR3 cells were grown to near confluence in 175 cm² flasks (Corning, NY, USA) and then placed into serum-free media overnight. Cells were detached in HBSS containing 5 mM EDTA and recovered by centrifugation at 150 g for 5 minutes. The cell pellet was washed twice in ice-cold HBSS containing 2 mM CaCl₂ and 2 mM MgCl₂ and cells were adjusted to a final concentration of 10⁶/ml in the same buffer. Cells (100 µL) were mixed with 100 nM E2-FITC and seeded into 96 well conical V-shaped bottom microtiter plates on ice containing an equal volume of either HBSS or various concentrations of 17b-E2, partial PROTAC, or E2-PROTAC. Samples were then incubated on ice for 30 min. Cells were pelleted by centrifugation at 4° C. and washed once in HBSS with cations and analyzed in an Aurora FCM instrument (Cytek Biosciences, USA). At least 10,000 events per sample were analyzed using forward scatter versus side scatter dot-plot gating to resolve the primary cell population. The fluorescence intensity of cells in the fluorescein isothiocyanate (FITC) channel for each sample was recorded in log mode. Each condition was performed in triplicate and reported as median intensity fluorescence plus or minus standard deviation.

ER binding was measured using cytosolic fractions prepared from MCF-7 cells by fluorescence polarization. MCF-7 (10⁶ cells) were detached in EDTA, cell homogenates were prepared using a Dounce homogenizer and subcellular fractions were isolated by differential centrifugation as previously described (Filardo et al, 2002). The protein concentration of each fraction was determined by bichinchoninic acid (BCA) assay (Pierce ™ BCA protein assay kit, Thermo Fisher). For saturation binding assay of the probe, different concentrations of cytosolic fraction were diluted in assay buffer (10 mM Tris-HCl, pH 7.4; 50 mM KCl, 10% glycerol, 0.1 mM dithiothreitol (DTT), 1 ug/mL BGG, 10 nM protease inhibitor cocktail (Roche) to a final volume of 200 uL containing 10 nM Ez-FITC. The mixture was incubated at ambient temperature for 1 hour and interaction of the probe with cytosolic protein was determined by fluorescence polarization (FP). For competitive binding assay, an aliquot of cytosolic fraction and E₂-FITC (10 nM final concentration) was mixed and incubated at 4° C. for 1 hour. Then different concentrations of drugs and controls were added to the mixture. The mixture was incubated for an additional hour and then subjected to FP. All samples were measured in triplicate. Negative controls (only cytosolic fraction or only E₂-FITC) and positive control (E2-FITC and cytosolic fraction at 100% binding without treatment) were included. Relative binding affinities were expressed as the percentage of FP recorded from treated wells compared to the positive control. FP values in millipolarization units (mP) were measured at ex/em wavelength of 485 nm/530 nm respectively using the Spectra Max plus 384 Microplate Spectrophotometer (Molecular Devices, Sunnyvale, CA) in 96-well, black, flat-bottom microplates (Greiner Bio-One North America, Inc., Monroe, NC). K_{d} values of the probe were determined by nonlinear regression fitting of the saturation curves, while IC50 values of treatment were determined by nonlinear regression fitting of the competition curves.

Cell cytotoxicity assays. Cell viability was measured following E2-PROTAC treatment using the PrestoBlue Viability kit according to the manufacturer’s suggested protocol (Thermo Fisher Scientific). In brief, breast cancer cells were seeded in 96 well-plates at a density of 10⁴ cells/well in growth media. On the following day, the contents of the 96 well plates were aspirated and replaced by treatment at different concentrations (ranging from 10⁻⁹ to 10⁻³ M), followed by the addition of media to bring the total volume to 200 µL/well. The untreated control group was incubated with 200 µL/well of fresh media. One day later, the media were aspirated and replaced by 90 µL media + 10 µL Prestoblue reagent, followed by incubation at 37° C. for 1 h. The fluorescence was excited at 560 nm and emission was recorded at 590 nm using a Spectra Max plus 384 Microplate Spectrophotometer (Molecular Devices, Sunnyvale, CA). Relative cell viability values were expressed as the percentage of the fluorescence recorded from wells containing treated cells compared to the control wells containing untreated cells.

Cell cycle analysis. Cells were seeded in 6 well-plates at a density of 2.5×10⁵ cells/well and then left untreated or exposed to partial PROTAC, UI-EP001 or UI-EP002 for 24 hours. Following treatment, cells were harvested by trypsinization and washed twice in ice-cold PBS. Cells were then fixed in 70% ethanol at 4° C. for 30 minutes. Fixed cells were pelleted by centrifugation at 230 g for 5 minutes and the cell pellet was incubated in Krishan’s solution (3.8 mM sodium citrate (Fisher Scientific), 0.014 mM propidium iodide (AnaSpec, Fermont, Ca), 1% NP-40 (Sigma) and 2.0 mg/mL RNase A (Fisher Scientific)) for 30 minutes at 37° C. Cells were then centrifuged and washed in PBS prior to analysis using a FacScallibur flow cytometer. The data from flow cytometry were subjected to further analysis by CellQuest software version 3.3. DNA histograms generated indicate the fractions of the cell population in the sub-G1, G0-G1, S, or G2/M phase of the cell cycle.

Statistical analysis. All data were analyzed using GraphPad Prism (GraphPad Software, San Diego, CA). The normality and homogeneity of variance of all data were analyzed by one-way analysis of variance (ANOVA). Each experiment was performed in triplicate and differences were considered significant if P < 0.05.

### Results

### Molecular Docking Analysis of ERa or GPER1 Interaction With E2-PROTAC

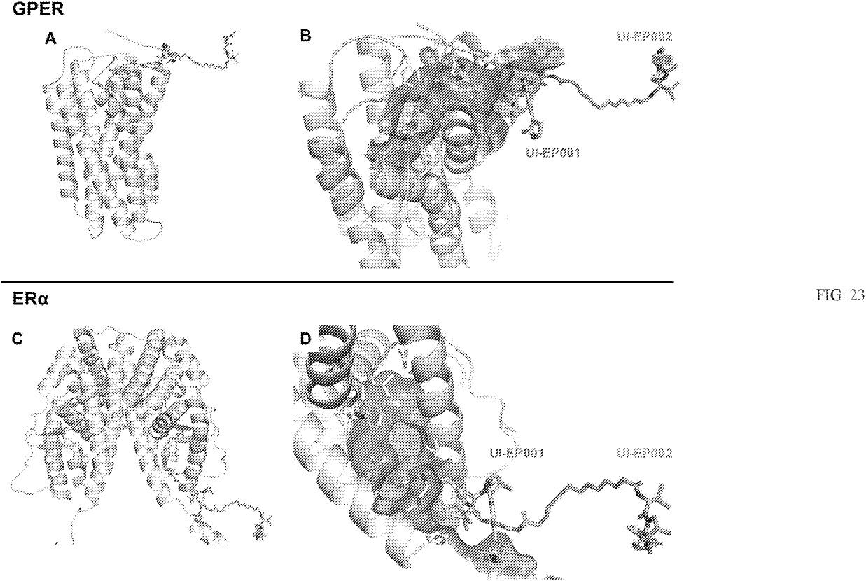

Previous studies of the GPER binding pocket and its interactions with E2 indicate that E2 can be modified using a PROTAC strategy and still maintain its binding efficacy (Amatt et al., 2012; Amatt et al., 2013). Based on those homology modeling studies, the 17C-hydroxyl group is postulated to point outward of the GPER binding pocket between TM1 and TM7 and interact with N118^{2.62} and H307^{7.37}. Previous studies have shown that the attachment of a fluorophore at the 17C does not negatively impact GPER activity (Filardo et al., 2007). Therefore, two different PROTAC molecules were designed in silico to have a von Hippel Lindau, VHL, E3 ligand (VHL⅟VHL032) attached to the 17-hydroxyl group via a polyethylene glycol (PEG) linker. An ester linkage to the 17-hydroxyl group was chosen for synthetic ease and two different lengths of linkers were chosen to explore the possible spatial constraints for GPER-PROTACs. The first molecule, UI-EP001, was designed with a 14-atom linker between E2 and VHL1, and the second molecule, UI-EP002, was designed with a 32-atom linker between E2 and VHL1. The linker length for UI-EP001 was based on reported literature, where 13-14-atom linkers resulted in high degradation rates and selectivity of the target receptor, while the more elongated linker in UI-EP002 helped to increase the overall hydrophilicity and flexibility of the compound. Docking studies for UI-EP001 and UI-EP002 with GPER1 show that the original E2 binding pocket is maintained and that the linker exits the binding pocket between TM1 and TM7 (FIG. 23). Based upon crystal structures of known PROTACs with their protein targets, both linker lengths would allow for UI-EP001 and UI-EP002 to interact with VHL E3 ligases while bound to GPER1 (Farnaby et al., 2019; Gadd et al., 2017).

While it is known that modification of the 17C-position of E2 is tolerated for GPER binding (Thomas et al., 2005), it is unclear if the designed PROTAC molecules would favorably bind with ERα. Therefore, additional docking studies for UI-EP001 and UI-EP002 with ERα were performed using a crystal structure of E2 bound to an ERα ligand-binding domain (PDB:1A52). All the known interactions between E2 and ERα are maintained for both UI-EP001 and UI-EP002. In addition, the linker length of both molecules is sufficiently long enough for the VHL1 portion to interact with VHL E3 ligases.

The synthetic detail of partial PROTAC (compound 7) is schematized in FIG. 24 (scheme 1). Linker 2 was prepared from triethylene glycol following a two-step scheme 1A. VHL E3 ligase ligand (VH032) was synthesized following previously published literature (scheme 1B) (Steinebach et al., 2019; Madak et al., 2017). Palladium-catalyzed cross-coupling between 4-bromobenzonitrile and 4-methylthiazole was carried out to achieve cyanide 3, which was subsequently treated with cobalt (II) chloride and sodium boron hydride to give primary amine 4. Compound 7 was achieved from 4 after amide coupling with Boc-Hyp-OH, Boc-Tle-OH and linker 2, subsequently. Conjugation between E2 and compound 7 was carried out under Steglich conditions to give UI-EP001 or compound 8 (scheme 2). Ul-EP002 (compound 10) was prepared by introducing a PEG8 linker in between E2 and compound 7. First, E2 and Fmoc-NH-PEG8-CH2CH2COOH underwent Steglich esterification, followed by treatment with trimethylamine to remove the Fmoc group and give compound 9. Finally, compound 10 was achieved from the conjugation between compound 9 and compound 7 with careful monitoring by HPLC for 72h (scheme 3).

### E2-PROTACs Exhibited High-Affinity Specific Binding to Plasma Membrane and Intracellular Estrogen Receptors

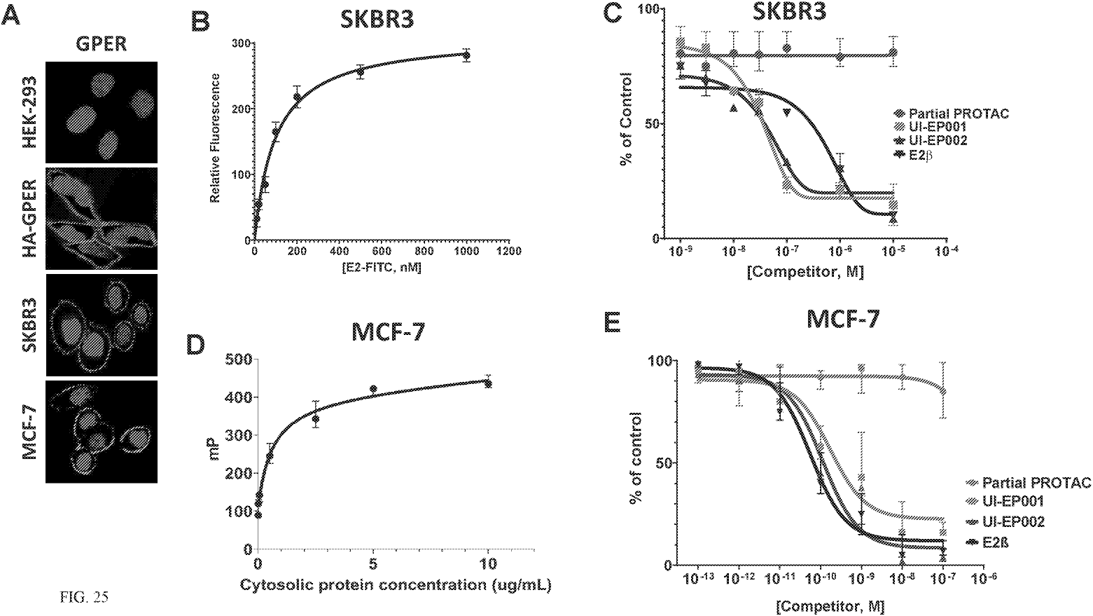

GPERs and ERs each bind E2 with high affinity (Prissnitz et al., 2015). Yet each estrogen receptor resides in distinct subcellular compartments, which has important implications regarding their relative ability to interact with E2-PROTACs. GPER exhibits all of the hallmarks of a plasma membrane receptor despite the fact that the bulk of the receptor is expressed in intracellular membranes (Filardo et al., 2007). In support of its role as a plasma membrane receptor, prior studies have measured specific binding of ³H-E2 in sucrose density gradient enriched membrane homogenates suggesting a plasma membrane binding site (Thomas et al., 2005). To directly evaluate the binding of UI-EP0001 and UI-EP002 to GPER on the plasma membrane, competitive binding assays were performed using intact SKBR3 cells that express GPER, but not ERa or ERb, using cell impermeant E2-FITC as a fluoro-tracer (FIG. 25). GPER specific binding activity of E2-FITC was measured by differential saturation analysis, subtracting the binding activity of cells that express or do not express GPER on the cell surface (FIG. 25A). Maximum GPER specific binding was reached near 100 nM E2-FITC (FIG. 25B) and this concentration was selected for competitive binding experiments (FIG. 25C). The concentration of E2 necessary to displace 50% of E2-FITC (IC₅₀) was calculated as the dissociation constant (Kd) for E2 at 100 ± 1.5 nM of 17b-E2. This is similar to the IC₅₀ value of 300 nM for E2 reported previously (Cao et al., 2017). By comparison, the IC₅₀ values of UI-EP001 and UI-EP002 were nearly 3-fold greater (RBA= 330%) than that measured for E2 (30.2 ± 0.9 nM and 30.2 ± 2.3 nM, respectively) (Table 1). This may, in part, be explained by the structural homology shared between E2-FITC and E2-PROTAC, which each contain a 17C-substituted hydroxyl. In contrast, a partial PROTAC consisting of the chemical spacer for UI-EP001 linked to the VHL E3 ubiquitin ligase recognition motif but lacking its E2-targeting domain was unable to compete for E2-FITC binding even at concentrations as high as 10 mM (FIG. 25C).

TABLE 1

Binding efficacy of UI-EP001 and UI-EP002 for GPER and ERs

| | | | | |
|---|---|---|---|---|
| Compounds | IC50 (nM) | RBA% | IC50 (nM) | RBA% |
| Estradiol | 100 ± 1.5 | 100 | 5.6 ± 1.4 | 100.0 |
| UI-EP001 | 30.2 ± 0.9 | 331.1 | 17.2 ± 8.2 | 32.0 |
| UI-EP002 | 30.2 ± 2.3 | 331.1 | 11.4 ± 3.3 | 49.0 |
| Partial PROTAC | NA | NA | NA | NA |

Abbreviations: IC50, concentration of 50% inhibition; RBA, relative binding affinity calculated as the ratio of the IC50 value of 17β-E2 divided by the IC50 of the test compound. All IC50 values were measured from triplicate samples and are reported as the mean ± SD; NA, not achieved.

TABLE 2

IC50 values for UI-EP001 and UI-EP002 in cell viability assays

| Cell line | IC50 (µM) | IC50 (µM) |
|---|---|---|
| MCF-7 | 9.0 ± 1.2 | 17.0 ± 1.6 |
| SKBR3 | 10.9 ± 1.2 | 11.1 ± 1.6 |
| HCC1806 | 34.9 ± 1.2 | 7.4 ± 1.3 |
| MDA-MB-231 | >100.0 | >100.0 |

Abbreviations: IC50, concentration of 50% inhibition; All IC50 values were measured from triplicate samples and are reported as the mean ± SD.

In contrast to GPER, ERa and ERb are intracellular receptors that are concentrated in the cytoplasm and in the nucleus. Thus, the binding activities of UI-EP001 and UI-EP002 ERs were evaluated in cytosolic fractions isolated from MCF-7 breast cancer cells using fluorescence polarization (FP). A saturation analysis was performed using 10 nM E2-FITC and increasing concentrations of cytosolic protein (FIG. 25D). Based upon near saturation (80%) FP values, competitive binding assays were performed using 600 mg/mL of cytosolic protein and E2 showed half-maximal displacement, IC₅₀= 5.6 ± 1.4 nM) (FIG. 25E). By comparison, IC₅₀ values for UI-EP001 and UI-EP002 were calculated at 17.2 ± 8.2 nM and 11.4 ± 3.3 nM, respectively. These results demonstrate that UI-EP001 and UI-EP002 display a slightly weaker binding affinity compared with E2, with RBAs of 32% and 49%, respectively (Table 1). Collectively, these competitive binding assays performed on intact cells and cytosolic fractions show that UI-EP001 and UI-EP002 bind plasma membrane and intracellular estrogen receptors GPER with high affinity in the low nanomolar range.

### E2-PROTAC Reduces the Expression Level of Native And/or Recombinant Estrogen Receptors

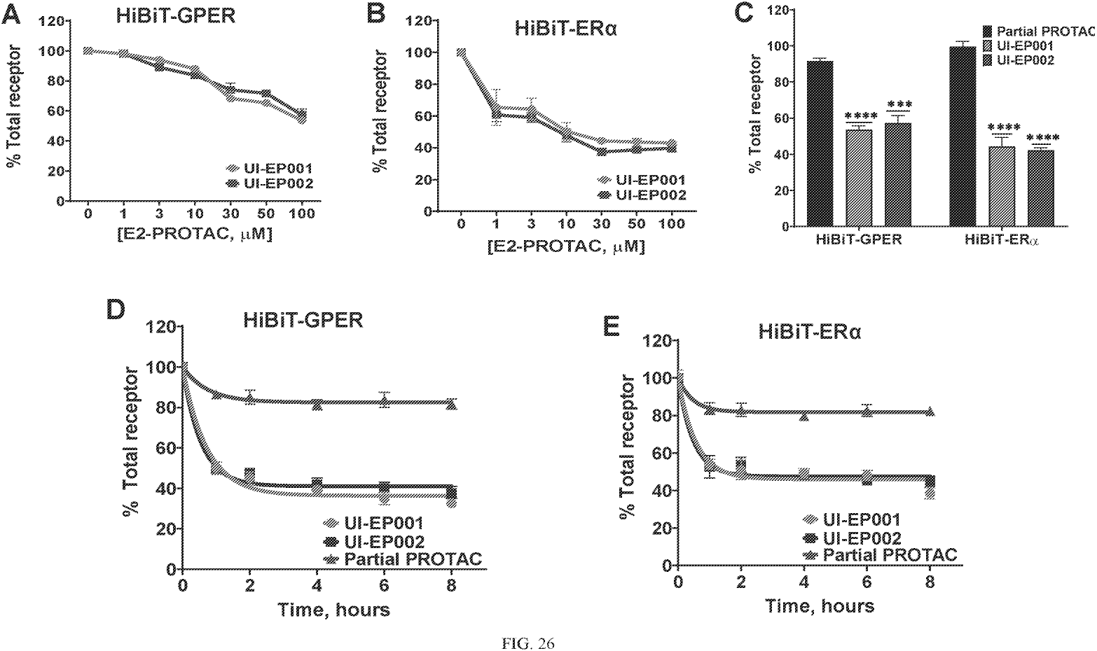

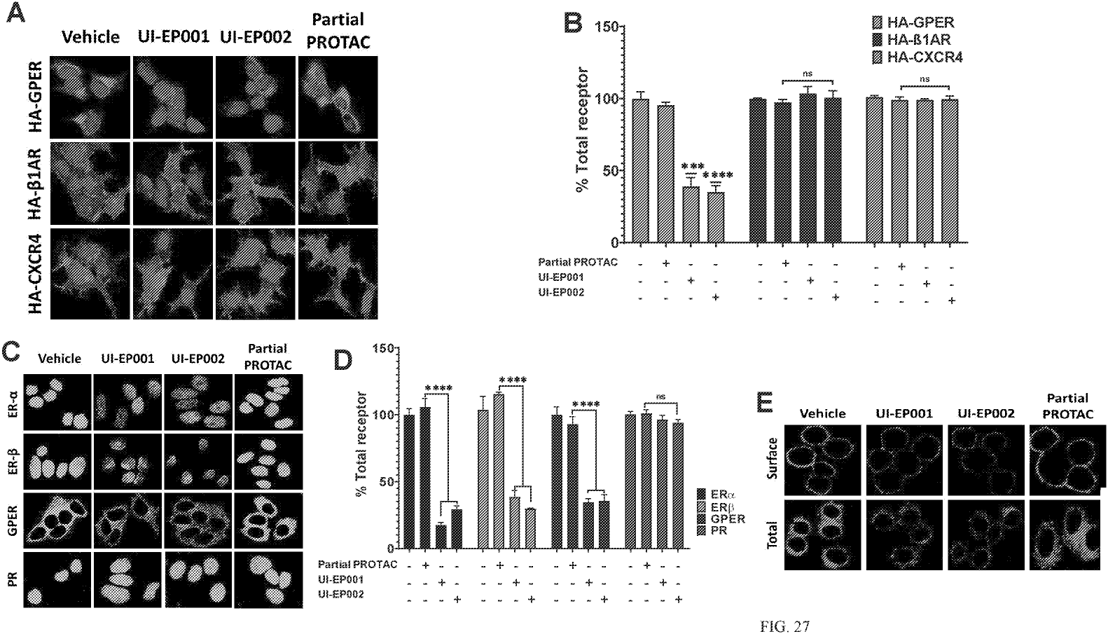

To evaluate the efficacy of UI-EP001 and UI-EP002 to degrade plasma membrane and intracellular estrogen receptors, a highly sensitive Nano-BiT binary luminescence complementation assay was employed that measures the interaction between HiBiT-tagged proteins and soluble Lg-BiT protein. Cells treated with UI-EP001 or UI-EP002 demonstrated a dose-dependent decrease of HiBiT-GPER or HiBiT-ERα (FIGS. 26A and B), while 100 µM of partial PROTAC did not show any significant decrease for either recombinant receptor within the tested concentration (FIG. 26C). From these assays, a DC₅₀ value (the concentration required for 50% protein degradation) was calculated for GPER (100 µM) and ERα (10-100 µM) respectively. Using these DC₅₀ concentrations, the kinetics of UI-EP001 and UI-EP002 degradation of HiBiT-GPER and HiBiT-ER were measured. Both UI-EP001 and UI-EP002 induced time-dependent reduction in expression levels of either recombinant GPER or ER, with 50% reduction measured as early as 1 h (FIGS. 26D and E). In contrast, 83% of the receptor was present following treatment of cells with partial PROTAC for 8 hrs.

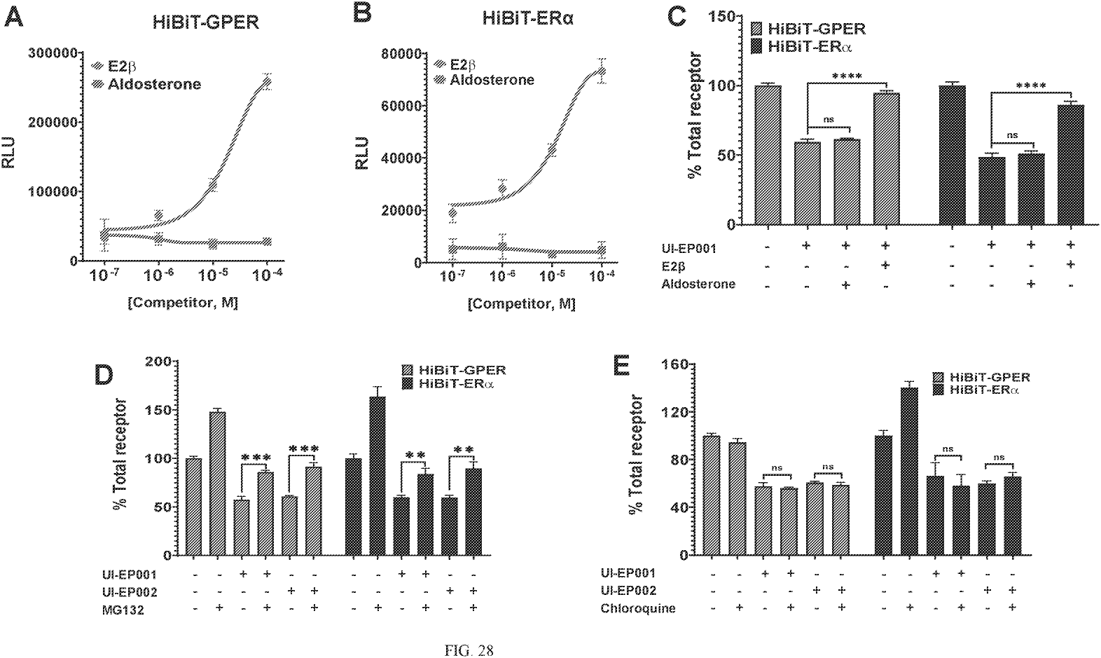

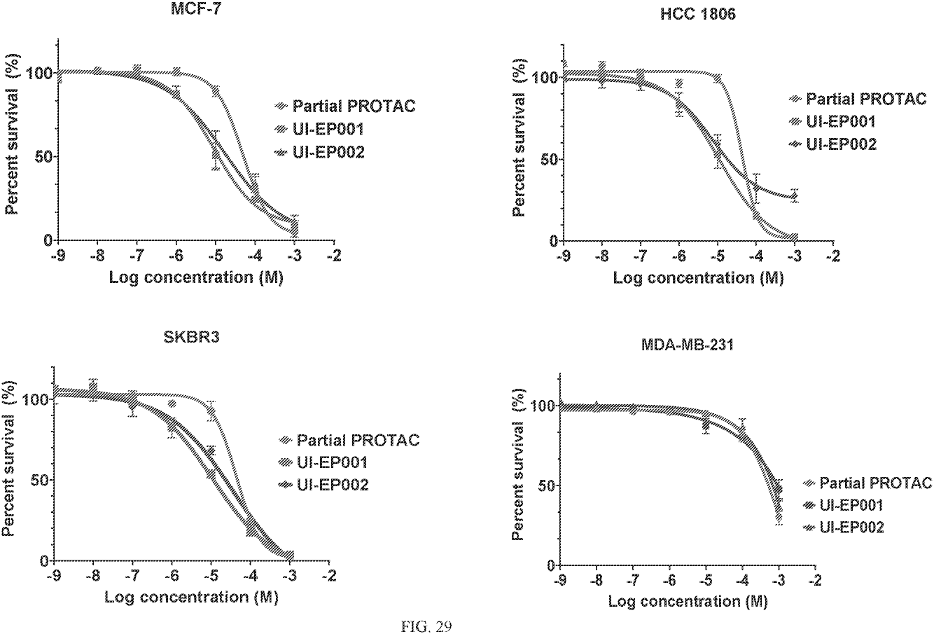

The specificity of UI-EP001 and UI-EP002 for GPER was determined by examining their capacity to degrade other GPCRs, including the b1-adrenergic receptor, b1AR and the chemokine C-X-C chemokine type 4 receptor, CXCR4 (FIG. 27A). For this purpose, the relative efficacy of UI-EP001 or UI-EP002 or partial PROTAC was evaluated for their efficacy to degrade HA-GPER, -b1AR, or -CXCR4 in stably transfected HEK293 cell lines. By immunofluorescent analysis using HA-specific antibodies, specific degradation of HA-GPER was measured without detectable off-target degradation of either HA-b1AR or HA-CXCR4 (FIGS. 27A and B). Next, the specificity of UI-EP001 and UI-EP002 to degrade endogenous plasma membrane and intracellular estrogen receptors was assessed using human MCF-7 breast cancer cells which express ERa, ERb, and GPER (FIGS. 27C and D). MCF-7 cells were exposed to either UI-EP001, UI-EP002, partial PROTAC for 1 hour or left untreated and then fixed, permeabilized and immunostained with specific antibodies for GPER, ERa, ERb or progesterone receptor, PR. Clear site-specific reduction was observed for nuclear ERα and ERβ and intracellular GPER (FIGS. 27C and D). In contrast, treatment of MCF-7 cells with partial PROTAC did not impact ERa, ERb, or GPER expression. Neither did UI-EP001 or UI-EP002 exhibit any detectable degradation of PR in MCF-7 cells further indicating the specificity of E2-PROTACs for estrogen receptors (FIG. 5D). Moreover, UI-EP001 and UI-EP002, but not partial PROTAC, reduced plasma membrane and total GPER in human SKBR3 breast cancer cells that are negative for ERa and ERb (FIG. 27E). Similarly, E2-specific PROTAC-mediated degradation was observed at the surface of MCF-7 cells. The specificity of UI-EP001 and UI-EP002 for its intended targets was further assessed by examining the capacity of exogenous E2 to inhibit E2-PROTAC-mediated ER/GPER degradation (FIGS. 28A and B). Cells transfected with ER-HiBiT or GPER-HiBiT were treated with 100 µM of UI-EP001 and UI-EP002 alone or in combination with increasing doses of E2β or aldosterone (100 nM-100 µM) for 1 hour (FIGS. 28A and B). While E2 effectively inhibited UI-EP001 and UI-EP002 degradation, degradation of either estrogen receptor target was not measured in the presence of the control steroid, aldosterone (FIG. 28C).

Taken together, these findings suggest that UI-EP001 and UI-EP002 function as estrogen receptor degraders (SERDs) that are capable of selectively degrading plasma membrane and intracellular estrogen receptors.

UI-EP001 and UI-EP002 degradation of ER and GPER occurs via the 26S- proteasome. Finally, it was assessed whether UI-EP001 and UI-EP002 could promote the degradation of their target receptors via the ubiquitin-proteasome pathway. To conduct these experiments, HEK293 cells transiently expressing HiBiT-tagged estrogen receptors were pretreated with the proteasome inhibitor, MG132, the lysosomotrophic agent, chloroquine, or vehicle prior to exposure to UI-EP001 or UI-EP002, and then total receptor expression was assessed as above. While MG132 reversed UI-EP001 or UI-EP002 degradation of either GPER or ERα, chloroquine had no such effect (FIGS. 28D and E). This result is consistent with the fact that PROTAC-induced protein degradation occurs as the result of receptor polyubiquitination and proteolysis at the 26S-proteasome. Significantly, these results suggest that our first generation E2-PROTACs; UI-EP001 and UI-EP002, interact with estrogen receptors that reside at the plasma membrane and in intracellular compartments to rapidly promote the degradation of membrane (GPER) and nuclear (ERs) estrogen receptors at the 26S-proteasome.

Collectively, these studies imply that simultaneous engagement of estrogen receptors (ERα and GPER) and VHL E3 ubiquitin ligase by UI-EP001 and UI-EP002 is crucial for effective degradation.

### E2-PROTAC Inhibits Breast Cancer Cell Growth by Inducing Cell Cycle Arrest

To evaluate the cytotoxic potential of the E2-PROTACs, the viability of four different human breast cancer cell lines with different estrogen receptor profiles was measured following treatment with UI-EP001, UI-EP002, or partial PROTAC (FIG. 29). In this study, the following were included: i) MCF-7 cells that express all three estrogen receptors (ERa⁺ ERb⁺, GPER⁺), ii) SKBR3 cells that overexpress her2/neu (ERa⁻, ERb^{-,} GPER⁺), iii) HCC1806 cells, which are triple negative and GPER-positive (ERa⁻, ERb⁻, GPER⁺) and iv) MDA-MB-231 cells, which are ERa-negative and express low levels of ERb and GPER and do not elicit either ER (Al-Badar et al., 2011) or GPER (Filardo et al., 2000)-dependent signaling activity (ERa⁻, ERb^{low}, GPER^{low}). Each breast cancer cell line was treated for 24 hours with various concentrations of E2-PROTAC or partial PROTAC and then cell viability was assessed using an MTS assay that measures mitochondrial oxido-reductase enzymes capable of converting a tetrazolium salt to a formazan dye (summarized in Table 2). A dose-dependent decrease in cellular viability was measured in all breast cancer cell lines that were treated with either UI-EP001 or UI-EP002. For UI-EP001, IC₅₀ values of 9.0, 10.9 and 34.9 mM were measured respectively in MCF-7, SKBR3 and HCC1806 breast cancer cell lines, which express GPER. Similar IC₅₀ values were measured for UI-EP002 in these three cell lines (17.0, 11.1 and 7.4 mM), respectively. In contrast, IC50 values for UI-EP001 and UI-EP002 were approximately 100-fold higher (> 100 mM) in MDA-MB-231 cells that express low concentrations of functional ERb or GPER, with >80% cell survivability. These IC₅₀ values were similar to those measured for cells treated with partial PROATAC. These data suggest that the elimination of membrane and intracellular estrogen receptors are critical factors in determining breast cancer cell viability.

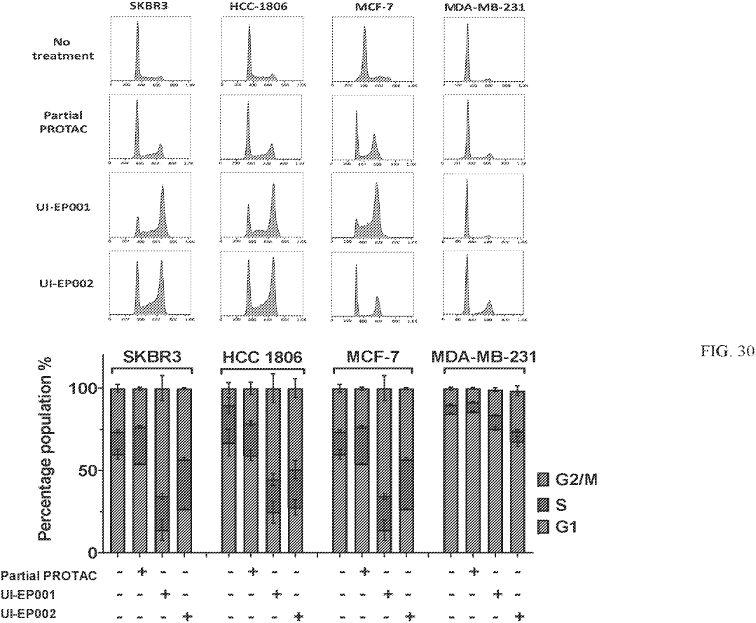

To investigate the biological effect associated with decreased cell viability, cell cycle analysis was performed on the same four cell lines using propidium iodide staining and measured by flow cytometry 24 hours after treatment of cells with 10 µM E2-PROTAC or partial PROTAC (FIG. 30). Untreated cells from all four cell lines showed a typical cell cycle distribution pattern with the majority of the cells in G1 and a small percentage of cells in either S or G2/M. Following treatment with either UI-EP001 or UI-EP002, a dramatic increase in the G2/M peak with a significant reduction in G1, was observed in MCF-7, SKBR3 and HCC1806 breast cancer cells. This shift was not observed in MDA-MB-231 cells that do not express ERa and express low amounts of functional ERb or GPER, suggesting that E2-PROTACs induce cell cycle arrest at the G2/M phase by reducing either or both estrogen receptors. As expected, cells treated with partial PROTAC displayed a much less significant shift in the percentage of the total cell population between G1 and G2/M. These in vitro results strongly suggest that the degradation of ERα and/or GPER has the potential to be effective in vivo for treating not only ER+ but also ER- breast cancer.

### Discussion

The carcinogenic effects of estrogen are manifested through cellular receptors that belong to the nuclear steroid hormone and G-protein coupled receptor superfamilies (Filardo et al., 2018). A number of ER-targeted PROTACs have been developed but they have not yet been tested for their capacity to degrade GPER. Here, proof-of-principle evidence is provided that a single estradiol-based PROTAC can target both receptor classes. Guided by in silico modeling and structure-activity relationship data, two first generation PROTACs (UI-EP001 and UI-EP002) were designed containing an estradiol-based targeting domain substituted at the 17C with ester-linked 14- or 32- atom length PEG spacers joined to the small molecule VHL E3 ubiquitin ligase recruitment motif; (S, R, S) AHPC. Each PROTAC exhibited a high binding affinity for nuclear ER and GPER and selective proteasome-dependent degradative activity for GPER and ERs with no measurable detectable off-target activity. Most importantly, UI-EP001 and UI-EP002, exhibited target-specific cytotoxicity and G2/M arrest, desirable in vitro characteristics for a first-generation pan-estrogen receptor PROTAC.

The PROTAC platform has been employed to selectively degrade a wide range of proteins that have therapeutic potential for human disease, including but not limited to; steroid hormone receptors (AR, ERa, ERRa, RAR) (Itoh et al., 2011; Peng et al., 2019; Schneekloth et al., 2008), Bromo- and Extra-Terminal (BET) family proteins (Lu et al., 2015; Winter et al., 2015), kinases (Bondesonet al., 2018; Lai et al., 2016), and microtubule-associated proteins (Tau, TACC3) (Silva et al., 2019). This group has recently been extended to include GPCRs (Li et al., 2020). The mere fact that PROTACs can efficiently target proteins with unique structural characteristics that are localized in distinct subcellular compartments demonstrates their exciting promise. However, the development of a PROTAC for a single protein target is not a trivial matter. Ultimately, target optimization is achieved by evaluating different targeting domains and/or warhead domains and making numerous fine adjustments that alter the length and composition of the chemical spacer as well as the overall stereochemistry of the entire hetero-bifunctional molecule. Theoretically, designing a single PROTAC capable of targeting two structurally and functionally distinct estrogen receptors with unique structures that reside in distinct physicochemical environments would pose a more challenging task but one that is necessary to treat ER+ breast cancers, since the majority of these tumors also express GPER. A broad assortment of ERa-PROTACs have been developed over the past decade but no single published study has demonstrated whether these compounds are also capable of targeting the highly related estrogen receptor, ERb, or the more recently described plasma membrane estrogen receptor, GPER.

Pioneering ER-PROTACs contained peptidic warhead domains and consequently showed low cell permeability and dependence upon cellular enzymatic activity to effectively recruit the E3 ubiquitin ligase SCF^{b-TCRP} (Sakamoto et al., 2003). More recent second-generation ER-PROTACs employ ER antagonists in their targeting domain and demonstrate high cell permeability and degradative capacity at low nanomolar concentrations (Hu et al., 2019: Denizu 2012; Okuhira et al., 2013). These latter PROTACs may also target GPER as well as ER but perhaps to a lesser extent due to the anticipated reduced RBA of E2 compared to ER antagonists for either receptor. Assessment of drug-target interaction by experimental methodology is a critical step for evaluating target specificity and selectivity. However, in general, the binding properties of ER-PROTACs have not been measured, and in many instances target selectivity has not been evaluated. Both UI-EP001 and UI-EP002 show high affinity for both target estrogen receptors with K_{d} values calculated in the low nanomolar range suggesting that the 17C substituted E2 targeting domain interacts well with the ligand contact sites on both GPER and ER. Using 17C substituted E2-FITC and intact SKBR3 breast cancer cells that express GPER with no detectable ERa or ERb by RT-PCR (Vladusic et al., 1998), K_{d} values of 30.2 nM were measured for UI-EP001 and UI-EP002. This is consistent with past evidence that 1, 3, 5(10)-estratriene-3, 17p-diol 17-hemisuccinate: BSA (E2-BSA) is capable of stimulating GPER-dependent activation of intracellular cAMP (Filardo et al. 2007). However, these findings are at odds with published work that showed that free E2, but not E2-BSA, was able to either displace ¹²⁵I-16a-iodo-E2 bound to recombinant ERa or ERb or induce a gel shift in electrophoretic mobility assays (Stevis et al., 1999). Free 17b-E2, UI-EP001 or UI-EP002 displaced E2-FITC bound to cytosolic fractions prepared from MCF-7 cell homogenates containing endogenous ERa and ERb with K_{d} values measured at 5.6 nM, 17.2 nM and 11.4 nM, respectively. The reasons for the discrepancy between the present findings and those of Stevis and colleagues are not clear but they may be related to both differences in the binding probe as well as the multi-valency of E2 for BSA relative to the 1:1 stoichiometry of E2 and FITC. One possibility is that the binding kinetics of E2-FITC for GPER are different compared to ¹²⁵I-16a-iodo-E2, which is influencing the displacements of the probes. Nonetheless, these data suggest that our first generation E2-PROTACs possess the most important characteristic of any new drug, the capacity to interact specifically and with high affinity to its intended target.

By their design, PROTACs offer the promise as cancer therapeutics of selective protein targeted-degradation. With the intent of demonstrating target specificity for a given PROTAC, the gold standard assay is the use of mass spectroscopic analysis to provide a comparative global readout of the proteome of treated and untreated cells (Beveridge et al., 2020). From a practical standpoint, target specificity can be best assessed by evaluating a difference in the survival of cancer cells that express or lack the cancer therapeutic target. This is particularly important for assessing a dual specificity PROTAC designed to selectively degrade ERs and GPER. In the present study, target selectivity was evaluated using immunofluorescent, biochemical and biological methods. By immunofluorescent analysis, we were able to show selective site-specific decreases in ERa and ERb within the nucleus and for GPER at both the cell surface and intracellularly. Degradation of intracellular GPER by UI-EP001 or UI-EP002 is consistent with our prior findings that showed GPER-dependent specific displacement of ³H-17b-E2 from crude membrane fractions as well as plasma membrane fractions enriched by sucrose density centrifugation (Thomas et al., 2005). Selectivity was demonstrated by the fact that no decrease was measured for PR, CXCR4 or b1AR in E2-PROTAC-treated cells. Likewise, by binary luminescence complementation, target selectivity was also demonstrated by showing that exogenous E2 but not aldosterone was capable of blocking E2-PROTAC-mediated degradation of HiBiT-GPER or HiBiT-ER. Finally, target selectivity for UI-EP001 and UI-EP002, was shown in biological assays where we compared their relative effects on viability and cell cycle distribution in various breast cancer cell lines expressing different complements of estrogen receptors. Decreased viability and G2/M arrest were observed in MCF-7 cells that express all 3 estrogen receptors but not in MDA-MB-231 cells that lack ERa and express low levels of functional ERb and GPER. Whether or not this was due to reduced ER or GPER, or both, is not clear in these cells. It is noteworthy that other investigators have evaluated the cytotoxicity of ER-PROTACs in ER-positive MCF-7, BT-474 and CAMA-1 breast cancer cell lines, and that these cells also express GPER (Kargno et al., 2019; Zhang et al., 2004). Either E2-PROTAC in this study induced cytotoxicity and G2/M arrest following treatment of ER-negative, GPER-positive SKBR3 and HCC-1806 breast cancer cell lines that represent her-2/neu overexpressing and TNBC tumor immunophenotypes. While different chemotherapeutic agents demonstrate cell cycle arrest at different stages of the cell cycle, the observation that E2-PROTACs induce G2/M arrest is consistent with a prior report that the androgen receptor (AR)-PROTAC, ARD-61 also induces G2/M arrest (Zhao et al., 2020). In some regards it may appear somewhat surprising that UI-EP001 or UI-EP002 induce cytotoxic effects and cell cycle arrest due to the fact that a complete loss of either ER or GPER was not observed. One possible explanation for these results may be related to the fact that GPCRs generally show a hyperbolic relationship between ligand occupancy and receptor responses. This relationship is termed “fractional occupancy” and suggest why it may not be necessary to eliminate the total amount of GPER at the plasma membrane to observe a significant reduction in receptor activity. More importantly, the present in vitro findings indicate that E2-PROTACs may have therapeutic benefit not only for breast cancers that express GPER but also for some ER-negative breast cancers that are considered to be untreatable by estrogen targeted therapy.

Arvinas, LLC has developed PROTACs targeting either the ER (ARV-471) or AR (ARV-110) that have shown promising success in preclinical mouse models of cancer. Both PROTACs are in phase I clinical trials (NCT03888612 and NCT04072952 in clinicaltrials.gov) for prostate and breast cancer, respectively (Flanagan et al., 2018; Neklessa et al., 2019). PROTACs have several advantages that make them more appealing than current SERDs for the treatment of breast cancer (Neklessa et al., 2019). Because PROTACs are catalytic in nature, they are ideally suited for overcoming one of the main limitations of endocrine therapy, acquired drug resistance by either overexpression or mutation (provided that the ubiquitination acceptor site is not altered). In fact, ARV-471 has been demonstrated to efficiently degrade clinically relevant ERα mutants (Y537S and D538G) in cell lines and in PDX models (Flanagan et al., 2018). The targeting domain of ARV-471 is undisclosed and proprietary so it is not clear whether it is capable of also targeting GPER. Likewise, a number of other ER-PROTACs have been developed and it is also not clear whether or not these PROTACs target GPER. Because GPER is: expressed in 60% of ER-positive breast cancers (Filardo et al., 2008) linked with clinical variables that predict advanced cancer, commonly expressed in ER-negative breast cancer and TNBC, and associated with resistance to anti-estrogen therapy, it is critical to develop cancer therapeutics that target GPER. A pan-estrogen receptor PROTAC, such as the E2-PROTACs, UI-EP001 and UI-EP002, may be ideally suited for this purpose and would complement existing anti-estrogen therapies.

### Example 3

### Schemes

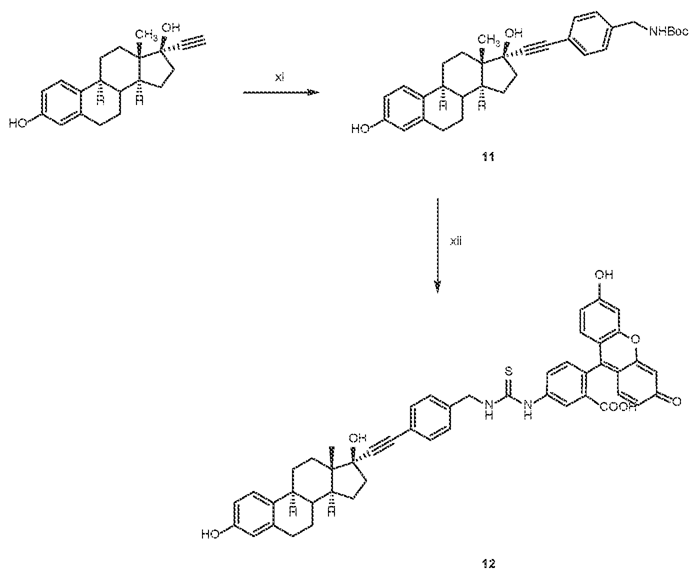

Scheme 1. Synthesis of E₂-FITC 12. Reagents and conditions: (xi) tert-butyl-4-iodobenzylcarbamate, Pd(Ph₃)₄, Cul, Et₃N, r.t, overnight; (xii) 1. TFA/DCM, rt, 2h: 2. FITC, pyridine, DMF, rt, overnight.

### Synthetic Procedures

Synthesis of UI-EP001 (8)

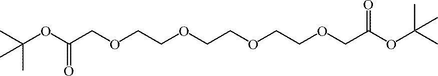

di-tert-butyl 3, 6, 9, 12 - tetraoxatetradecanedioate (1), A mixture of triethylene glycol (1.50 g, 1.36 ml, 10 mmol, 1 eq.), NaH 60% in mineral oil (800 mg, 20 mmol, 2 eq.) and tert-Butyl bromoacetate (4.50 g, 3.4 ml, 20 mmol, 2 eq.) were dissolved in 10 mL of dioxane. Reaction mixture was stirred overnight, followed by quenching with saturated NH₄Cl. The mixture was extracted with EtOAc and dried by Na₂SO4, then concentrated in vacuum. The product was applied to flash chromatography (Hexane:EtOAc = 8:2) to give compound 1 an colorless oil. Yield: 3.25 g, 8.60 mmol (92%). ¹H NMR (300 MHz, CDCb): δ 3.99 (s, 4 H), 3.72-3.66 (m, 16 H), 1.46 (s, 9 H). HRMS (ESI+) cald for C₁₈H₃₄O₈ [M + 1]⁺: 379.23, found 379.46.

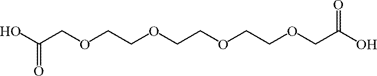 [SMILES: Cc1ncsc1-c1ccc(CNC(=O)C2C[C@@H](O)CN2C(=O)OC(C)(C)C)cc1]

di-tert-butyl 3, 6, 9, 12 - tetraoxatetradecanedioic (2). A solution of compound 1 (1.74 g, 4.62 mmol) in a 50% v/v trifluoroacetic acid (TFA) in DCM (6 mL per mmol) was stirred at r.t. for 2 h. TLC analysis (10% methanol in DCM) showed complete conversion of the starting material. Then, the reaction mixture was concentrated under vacuum and the crude product was freeze-dried to obtain the desired product 2 (quantitative yield). ¹H NMR (300 MHz, CDCl₃): δ 4.03 (s, 4 H), 3.85-3.42 (m, 16 H). HRMS (ESI+) cald for C₁₀H₁₈O₈ [M + 1]⁺: 267.10, found 267.56.

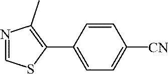

4-Methylthiazol-5-yl) benzonitrile (3). A mixture of 4-bromobenzonitrile (5.00 g, 27.5 mmol), 4-methylthiazole (4.98 mL, 54.7 mmol), KOAc (5.40 g, 55.0 mmol), and palladium acetate (62.0 mg, 0.27 mmol) were dissolved in 20 mL of DMAc. The mixture was heated to 120° C. overnight, cooled, and diluted with EtOAc. The solution was washed with brine, dried with Na₂SO₄ and concentrated. The resulting oil was applied to flash chromatography (Hexane:EtOAc = 9:1 -> 1:1 -> 1:9) . Compound 3 was obtained as a yellow solid (4.49 g, 22.5 mmol, 82%). ¹H NMR (300 MHz, CDCl₃) δ 8.76 (s, 1 H), 7.75 - 7.71 (m, 2 H), 7.60 - 7.55 (m, 2 H), 2.58 - 2.56 (m, 3 H). HRMS (ESI+) cald for C₁₁H₈N₂S [M + 1]⁺: 201.04, found 201.36.

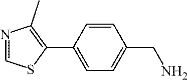

Methylthiazol-5-yl) phenyl) methanamine (4). Compound 3 (4.49 g, 22.5 mmol) was dissolved in 300 mL anhydrous MeOH. Cobalt chloride (CoCl₂) (4.39 g, 33.75 mmol) was added and the solution was cooled an ice bath for 30 min. NaBH₄ (5.22 g, 138 mmol) was added in fractions over the course of 20 min. The mixture was stirred for another 90 min and quenched with cold H₂O. The mixture was filtered, diluted in H₂O and extracted with EtOAc. The organic layer was dried with Na₂SO₄, filtered, concentrated, and purified via flash chromatography (DCM:MeOH = 99:1 -> 90:10, with 0.5 M Et₃N). Compound 4 was obtained as white powder (3.44 g, 16.88 mmol, 75%). ¹H NMR (300 MHz, DMSO-d₆) δ 8.94 (s, 1 H), 7.42 (m, 4 H), 3.77 (s, 2 H), 2.45 (s, 3 H). ¹³C NMR (300 MHz, DMSO-d₆) δ 152.23, 147.98, 145.01, 131.89, 129.95, 129.01, 127.35, 45.23, 15.98. HRMS (ESI+) cald for C₁₁H₁₂N₂S [M + 1]⁺: 205.07, found 205.77.

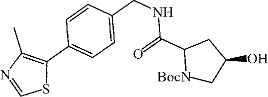

(4R)Hydroxy-N-(4-(4-methylthiazol-5-yl) benzyl)-Boc-pyrrolidine-2-carboxamide (5). A mixture of compound 4 (2.04 g, 10.00 mmol), Boc-Hyp-OH (2.31 g, 10.00 mmol), DIPEA (6.95 mL, 40.00 mmol), and HBTU (4.16 g, 11.00 mmol) were dissolved in 50 mL of anhydrous DMF. The mixture was stirred at room temperature overnight, then diluted with H₂O and extracted with EtOAc. The organic layer was washed with brine, dried with Na₂SO₄, and concentrated. The crude product was purified via flash chromatography (DCM:MeOH = 99:1 -> 90:10). Compound 5 was obtained as colorless oil (2.79 g, 6.7 mmol, 67%) ¹H NMR (300 MHz, CDCl₃) δ 8.69 (s, 1 H), 8.28 - 8.04 (m, 1 H), 7.45 - 7.32 (m, 4 H), 4.52- 4.44 (m, 3 H), 4.10 (t, 1 H), 3.10 - 3.00 (m, 1 H), 2.88 - 2.77 (m, 1 H), 2.54 (s, 3 H), 2.41 - 2.32 (m, 1 H), 2.07 - 1.93 (m, 1 H), 1.43 (s, 9 H).¹³C NMR (300 MHz, CDCl₃) δ 172.15, 153.88, 152.12, 148.02, 139.75, 131.52, 130.66, 129.05, 128.05, 77.25, 65.48, 55.98, 53.95, 42.16, 28.96, 14.86. HRMS (ESI+) cald for C₂₁H₂₇N₃O₄S [M + 1]⁺: 418.17, found 418.03.

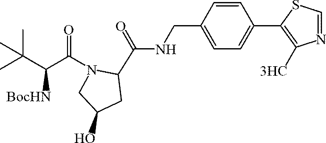

(4R)((S)-2-Boc-Amino-3,3-dimethylbutanoyl)-4-hydroxy-N-(4-(4-methylthiazol-5-yl) benzyl) pyrrolidine-2-carboxamide (6). Compound 5 (2.79, 6.7 mmol) was dissolved in 50% v/v TFA/DCM (6 mL per mmol) and stirred at room temperature for 2 h. The mixture was concentrated and used for next step without further purification. A mixture of last step product, Boc-L-tert-leucine (1.54 g, 6.7 mmol), HBTU (2.79 g, 7.37 mmol), and DIPEA (4.66 mL, 26.8 mmol) was dissolved in 50 mL of DMF. The mixture was stirred at room temperature overnight, diluted with H₂O, and extracted with EtOAc. The organic layer was washed with a saturated NaHCO₃ solution, brine, dried with Na₂SO₄, filtered, and concentrated. Crude product was purified with flash chromatography (DCM:MeOH = 99:1 -> 90:10), giving compound 6 as white powder (2.09 g, 3.95 mmol, 59% yield after two steps). ¹H NMR (300 MHz, DMSO-d₆) δ 8.98 (s, 1 H), 7.56 - 7.50 (m, 4 H), 4.91 - 4.86 (m, 4 H), 4.08 (s, 1 H), 3.36 - 3.31 (m, 1 H), 2.58 (s, 3 H), 2.34 - 2.30 (m, 1 H), 2.13 - 2.07 (m, 1 H), 1.20-1.02 (m, 18 H). ¹³C NMR (300 MHz, DMSO-d₆) δ 173.12, 170.15, 157.23, 153.48, 148.16, 139.68, 132.36, 130.12, 128.79, 125.56, 75.69, 65.26, 58.56, 55.23, 53.29, 42.56, 38.26, 36.18, 27.48, 25.42, 15.99. HRMS (ESI+) cald for C₂₇H₃₈N₄O₅S [M + 1]⁺: 531.26, found 531.49.

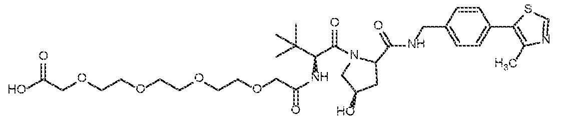

Partial PROTAC (7). Compound 5 (2.09, 3.95 mmol) was dissolved in 50% v/v TFA/DCM (6 mL per mmol) and stirred at room temperature for 2 h. The mixture was concentrated and used for next step without further purification. A mixture of last step product (1 eq), compound 2 (2.1 g, 7.9 mmol, 2 eq), HBTU (1.65 g, 4.30 mmol), and DIPEA (2.75 mL, 15.8 mmol) was dissolved in 50 mL of DMF. The mixture was stirred at room temperature overnight. As TLC showed complete reaction of starting material, the mixture was diluted in DCM and washed with brine, dried and concentrated. The product was purified by HPLC reverse phase column (Zorbax300SB-C18, 21.2×150mm, 5uCrt) to obtained desire product 7. Yield: (1.21 g, 46% after 2 steps). ¹H NMR (500 MHz, MeOD-d₄) δ 9.68 (s, 1 H), 7.56 - 7.50 (m, 4 H), 4.67. - 4.69 (t, 1 H), 4.58 - 4.55 (m, 2H), 4.43 - 4.40 (d, 1 H), 4.15 - 4.11 (m, 4 H), 4.08 - 4.05 (m, 17 H), 3.78 - 3.80 (m, 1 H), 3.85 - 3.71 (m, 1 H), 3.36 - 3.31 (m, 1 H), 2.58 (s, 3 H), 2.34 - 2.30 (m, 1 H), 2.13 - 2.07 (m, 1 H), 1.20-1.02 (m, 9 H). ¹³C NMR (500 MHz, MeOD-d₄) δ 172.97, 170.73, 170.31, 151.43, 147.69, 138.87, 132.01, 130.15, 129.00, 127.58, 70.93, 70.37, 70.24, 70.20, 69.77, 69.73, 69.66, 59.41, 56.75, 56.69, 50.39, 42.33, 37.53, 37.70, 29.26, 25.56, 14.40. HRMS (ESI+) cald for C₃₁H₄₄N₄O₁₀S [M + 1]⁺: 679.29, found 679.89.

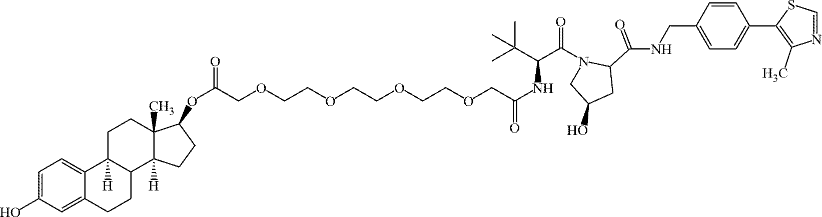

UI-EP001 (8). Compound 7 (300 mg, 0.46 mmol), was activated by M-Hydroxysuccinimide (NHS) (53 mg, 0.46 mmol) in 5 mL of DMF for 3 h, followed by the addition of 17β-estradiol (125 mg, 0.46 mmol) and 1-ethyl-3-(3-dimethylaminopropyl)carbodiimide (EDC) (71.3 mg, 0.46 mmol). Reaction mixture was stirred at room temperature for 48 h and monitored by reverse phase HPLC. The crude product was diluted in DCM:MeOH (90:10) and washed with brine, dried and concentrated. The product 8 was obtained by HPLC reverse phase column (Zorbax300SB-C18, 21.2×150 mm, 5uCrt). Yield (119 mg, 28%). ¹H NMR (500 MHz, MeOD-d₄) δ 9.64 (s, 1 H), 7.56 - 7.49 (m, 4 H), 7.09 - 7.07 (d, 1 H), 6.55 - 6.53 (m, 1 H), 6.48-6.49 (d, 1 H), 4.71 - 4.67 (t, 1 H), 4.58 - 4.55 (m, 2 H), 4.12 - 3.95 (m, 17 H), 3.86 - 3.84 (d, 1 H), 3.74 - 3.71 (m, 1 H), 3.68 - 3.65 (t, 1 H), 2.78 - 2.77 (m, 2 H), 2.57 (s, 3 H), 2.32 - 2.30 (m, 2 H), 2.14 - 1.95 (m, 4 H), 1.74 - 1.67 (m, 1 H), 1.54 - 1.18 (m, 7 H), 1.15 (s, 9 H), 0.78 (m, 3 H). ¹³C NMR (500 MHz, MeOD-d₄) 6 173.12, 170.15, 157.23, 153.48, 148.16, 139.68, 132.36, 130.12, 128.79, 125.56, 75.69, 65.26, 58.56, 55.23, 53.29, 42.56, 38.26, 36.18, 27.48, 25.42, 15.99. HRMS (ESI+) cald for C₅₀H₆₈N₄O₁₁S [M + 1]⁺: 933.46, found 933.23.

### Synthesis of Ul-EP002 (10)

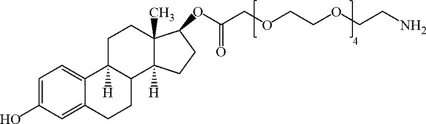

Compound (9). Fmoc-NH-PEG8-CH₂CH₂COOH (300 mg, 0.3 mmol) was activated by N-Hydroxysuccinimide (NHS) (34.5 mg, 0.3 mmol) in 1 mL of DMF for 3 h, followed by the addition of 17β-estradiol (81.6 mg, 0.3 mmol,) and EDC (46.6 mg, 0.3 mmol). Reaction mixture was stirred at room temperature for 72 h and monitored by reverse phase HPLC. Crude product was purified by HPLC reverse phase column (Zorbax300SB-C18, 21.2×150 mm, 5uCrt) to give compound 9 Yield (41 mg, 16%). ¹H NMR (500 MHz, MeOD-d₄) δ 7.12 - 7.11 (d, 1 H), 6.59 - 6.57 (m, 1 H), 6.52-6.51 (d, 1 H), 4.26 - 4.24 (m, 1 H), 3.77 - 3.74 (t, 2 H), 3.71 - 3.56 (m, 31 H), 3.34 - 3.32 (m, 2 H), 2.83 - 2.80 (m, 2 H), 2.59 - 2.56 (t, 2 H), 2.36 -2.31 (m, 1 H), 2.17 - 2.14 (m, 1 H), 2.09 - 2.05 (m, 1 H), 2.01 - 1.97 (m, 1 H), 1.92 - 1.88 (m, 1 H), 1.59 -1.19 (m, 8 H), 0.81 (s, 3 H). ¹³C NMR (500 MHz, MeOD-d₄) δ 171.62, 152.12, 141.58, 138.85, 135.04, 128.85, 125.00, 123.42, 122.38, 117.17, 112.27, 109.95, 78.73, 67.76, 67.74, 67.67, 67.59, 67.53, 67.14, 64.07, 63.81, 47.54, 40.59, 38.02, 36.76, 34.26, 32.13, 26.94, 26.93, 26.88, 24.75, 23.83, 20.25, 7.90 HRMS (ESI+) cald for C₅₁H₆₉NO₁₃ [M + 1]⁺: 905.11, found 905.19.

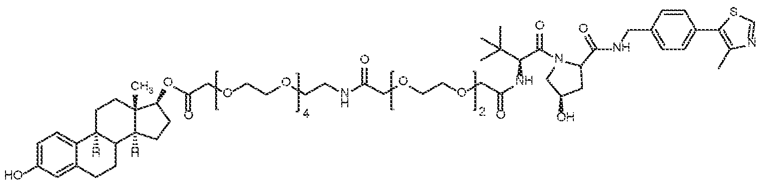

UI-EP002 (10). The Fmoc group of Compound 9 (35.0 mg, 0.39 mmol) was removed by Et₃N (1 mL) in DMF (5 mL). Et₃N was removed from the reaction mixture by rotavap, followed by the addition of the partial PROTAC 7 (264.4 mg, 0.39 mmol), HATU (178.6 mg, 0.47 mmol) and DIPEA (0.271 mL, 1.56 mmol). Reaction mixture was stirred at room temperature for 48 h and monitored by reverse phase HPLC. The product 10 was obtained by HPLC reverse phase column (Zorbax300SB-C18, 21.2×150 mm, 5uCrt). Yield (26 mg, 52%). ¹H NMR (500 MHz, MeOD-d₄) δ 9.69 (s, 1 H), 7.57 - 7.51 (m, 4 H), 7.12 - 7.11 (d, 1 H), 6.59 - 6.57 (m, 1 H), 6.52 - 6.51 (d, 1 H), 4.71 - 4.68 (m, 1 H), 4.59 - 4.56 (d, 1 H), 4.44 - 4.40 (d, 1 H), 4.26 -4.24 (m, 1 H), 4.09 (s, 1 H), 3.88 - 3.86 (m, 1 H), 3.77 - 3.74 (t, 2 H), 3.71 - 3.56 (m, 47 H), 3.36 - 3.32 (m, 6 H), 2.83 - 2.23 (m, 2 H), 2.59 - 2.56 (m, 5 H), 2.36 - 2.31 (m, 1 H), 2.21 - 2.14 (m, 1 H), 2.10 - 2.03 (m, 1 H), 2.01 - 1.97 (m, 1 H), 1.92 - 1.88 (m, 1 H), 1.76 - 1.70 (m, 1 H), 1.43 - 1.30 (m, 8 H), 1.15 (s, 9 H), 0.81 (s, 3 H). ¹³C NMR (500 MHz, MeOD-d₄) δ 174.01, 167.15, 154.52, 143.97, 141.25, 137.43, 131.25, 127.39, 126.77, 125.81, 124.81, 124.78, 119.56, 114.67, 112.34, 81.12, 70.16, 70.13, 69.98, 69.93, 69.54, 66.47, 66.21, 49.94, 42.98, 40.41, 39.16, 34.52, 29.33, 29.32, 29.17, 27.14, 26.22, 22.64, 10.30. HRMS (ESI+) cald for C₆₈H₁₀₃N₅O₂₀S [M + 1] ⁺: 1342.69, found 1342.15.

### Synthesis of E₂-FITC (12)

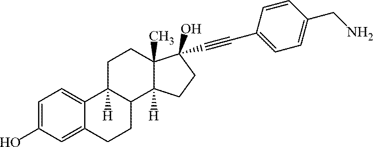

Compound (11). 17α-ethynylestradiol (592 mg, 2 mmol) was added to a mixture of 4-(t-butyloxycarbonylaminomethyl)iodobenzene (778 mg, 2.33 mmol), tetrakis(triphenylphosphine)palladium Pd(Ph₃)₄ (5 mole %), and Cul (5 mole %) in Et₃N (20 ml) under argon atmosphere. The reaction mixture was stirred at room temperature overnight, then solvent was reduced under vacuum. Crude product was purified by flash chromatography (Hexane:EtOAc = 20:80 -> DCM:MeOH = 90:10) giving yellow powder product. Yield (756 mg, 78%). ¹H NMR (300 MHz, MeOD-d₄) δ 7.43 - 7.41 (m, 2 H), 7.28 - 7.16 (m, 2 H), 6.67 - 6.64 (m, 1 H), 6.59 - 6.58 (d, 1 H), 4.71 - 4.67 (t, 1 H), 4.33 - 4.31 (d, 1 H), 2.83 (m, 2 H), 2.43 - 1.72 (m, 15 H), 1.57- 1.38 (s, 9 H), 0.94 (s, 3 H). ¹³C NMR (500 MHz, MeOD-d₄) δ 155.12, 143.25, 131.59, 130.98, 125.16, 124.33, 113.25, 113.21, 95.23, 84.03, 77.69, 62.22, 49.96, 45.11, 42.68, 35.66, 33.22, 27.22, 26.45, 25.56, 24.21, 13.56. HRMS (ESI+) cald for C₃₂H₃₉NO₃ [M + 1] ⁺; 486.29, found 486.85.

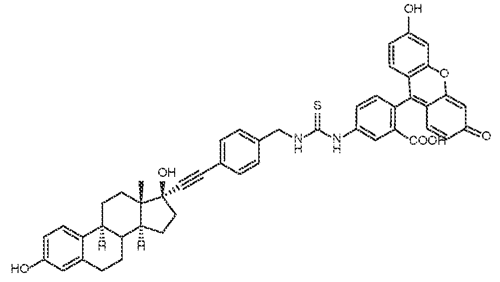

E2-FITC (12). A solution of compound 11 (485 mg, 1 mmol) in 50% v/v TFA/DCM was stirred at r.t. for 2 h. After TLC showed complete conversion, the mixture was co-evaporated with DCM 5-6 times to remove TFA. The residue was redissolved in DMF (5 mL), followed by the addition of FITC (429 mg, 1.1 mmol) and pyridine (0.25 mL). The reaction mixture was stirred in dark at room temperature overnight. After drying, the crude product was purified by flash chromatography (CHCl₃:MeOH = 95:5 -> 90:10) to afford compound 12 as yellow oil (355 mg, 45%). ¹H NMR (300 MHz, MeOD-d4) δ 10.23 (s, 1 H), 8.05 (m, 1 H), 7.83 - 7.79 (m, 1 H), 7.43 - 7.41 (m, 2 H), 7.27 (m, 2 H), 6.68 - 6.53 (m, 8 H), 4.70 (t, 1 H), 4.32 (m, 1 H), 2.85 (m, 2 H), 2.40 -1.53 (m, 15 H), 0.93 (s, 3 H). HRMS (ESI+) cald for C₄₈H₄₂N₂O₇S [M + 1]⁺: 791.27, found 791.28.

### REFERENCES

Achinger-Kawecka et al., Nat. Commun., 11:320 (2020).

Al-Bader et al., Exp. Ther. Med., 2:537 (2011).

Anderson et al., J. Clin. Oncol., 28:232 (2010).

Arnatt & Zhang, Computational approaches to nuclear receptors, 117, doi:10.1039/9781849735353-00117 (2012).

Arnatt & Zhang, Mol. Inform., 32:647 (2013).

Augusto et al., Endocr. Relat. Cancer, 25:R283 (2018).

Barone et al., Clin. Cancer Res., 16:2702 (2010).

Beveridge et al., Nat. Commun., 11:742 (2020).

Bondeson et al., Cell Chem. Biol., 25:78 (2018).

Brzozowski et al., Nature, 389:753 (1997).

Callige & Richard-Foy, Nucl. Recept. Signal, 4:e004 (2006).

Cao et al., Environ. Sci. Technol., 51:11423 (2017).

Chan et al., lnt. J. Cancer, 146:1674 (2020).

Cheng et al., J. Biol. Chem., 286:22441 (2011).

Cheng et al., Molec. Cell Endocrinol., 382:950 (2014).

Clarke et al., Pharmacol. Rev., 53:25 (2001).

Cosman et al., Thromb. Res., 116:1 (2005).

Cyrus et al., Mol. Biosyst., 7:359 (2011).

DeLeon et al., J. Lipid Res., 61:767b (2020).

De Placido et al., Lancet Oncol., 19:474 (2018).

deConinck et al., Mol. Cell Biol., 15:2191 (1995).

Demizu et al., Bioorg. Med. Chem. Lett., 22:1793 (2012).

Dragovich et al., Bioorg. Med. Chem. Lett., 30:126907 (2020).

Fan, J. & Liu, K. (Google Patents, 2020).

Fan et al., Future Med. Chem., 7:1511 (2015).

Farnaby et al., Nat. Chem. Biol., 15:672 (2019).

Filardo & Thomas, Endocrinol., 153:2953 (2012).

Filardo et al., Clin. Cancer Res., 12:6359 (2006).

Filardo et al., Endocrinol., 148:3236 (2007).

Filardo et al., Mol. Endocrinol., 14:1649 (2000).

Filardo et al., Molec. Endocrinol., 16:70 (2002).

Filardo, J. Steroid Biochem. Mol. Biol., 176:38 (2018).

Filardo, J. Steroid Biochem. Mol. Biol., 80:231 (2002).

Filardo et al., Clin. Cancer Res., 12:6359 (2006).

Filardo et al., Steroids, 73:870 (2008).

Filardo et al., Mol. Endocrinol., 14:1649 (2000).

Flanagan et al., In: Proceedings of San Antonio Breast Cancer Meeting (2018).

Flanagan et al., ARV. 1000:3 (2018).

Gadd et al., Nat. Chem. Biol., 13:514 (2017).

Galdeano et al., J. Med. Chem., 57:8657 (2014).

Garcia-Becerra et al., Int. J. Mol. Sci., 14:108 (2012).

Gaudet et al., Mol. Cell Endocrinol., 418:207 (2015).

Germain, Endocrinol. Metab. Clin. North Am., 40:473 (2011).

Giuliano et al., Breast, 20:S42 (2011).

Gu et al., Bioessays, 40:e1700247 (2018).

Guignet et al., Nat. Biotechnol., 22:440 (2004).

Haque & Desai, Front Endocrinol (Lausanne), 10:573 (2019).

Hollern et al., Br. Ca. Res. Treat, 174:143 (2019).

Hu et al., J. Med. Chem., 62:1420-1442, doi:10.1021/acs.jmedchem.8b01572 (2019).

Hu et al., Oncol. Lett., 9:1495 (2015).

Huang & Dixit, Cell Res., 26:484 (2016).

Ignatov et al., Breast Cancer Res Treat., 128:457 (2011).

Ishida et al., SLAS Discovery, 26:484 (2020).

Itoh et al., Bioorg. Med. Chem., 19:3229 (2011).

Jones et al., Breast Cancer Res., 14:R91, (2012).

Kargbo, ACS Med. Chem. Lett., 10:1367 (2019).

Kumar et al., J. Amino. Acids, (2011) Article ID 812540.

Lai et al., Angew Chem. Int. Ed. Engl., 55:807 (2016).

Lata et al., J. Am. Chem. Soc., 128:2365 (2006).

Lei et al., J. Cancer Metastasis Treat, 5: (2019).

Li et al., Acta Pharm. Sin. B., 10:1669 (2020).

Lu et al., Chem. Biol., 22:755 (2015).

Madak et al., Chemistry, 23:13875 (2017).

Marjon et al., Mol. Cancer Res., 12:1644 (2014).

Miller, Am. Soc. Clin. Oncol. Educ. Book, doi: 10.1200/EdBook_AM.2013.33.e37 (2013).

Molina et al., Front Endocrinol (Lausanne), 11:563165 (2020).

Nathan & Schmid, Oncol. Ther., 5:17 (2017).

Neklesa et al., Pharmacol. Ther., 174:138 (2017).

Neklesa et al., J. Clin. Oncol., 37:25 (2019).

Noel et al., Mol. Endocrinol., 23:349 (2009). PMID:19131510.

Okuhira et al., Cancer Sci., 104:1492 (2013).

Parl et al., Cancer Epidemiol Biomarkers Prev., 27:899 (2018).

Peng et al., ACS Med. Chem. Lett., 10:767 (2019).

Pepermans & Prossnitz, Steroids, 152:108493, (2019).

Petrie et al., Obstet. Gynecol. Int., 2013:472720 (2013).

Phadke et al., In: Proceedings of San Antonio Breast Cancer Meeting (2020).

Pike et al., Structure, 9:145 (2001).

Prossnitz & Arterburn, Pharmacol. Rev., 67:505 (2015).

Prossnitz & Hathaway, J. Steroid Biochem. Mol. Biol.. 153:114 (2015).

Prossnitz et al., Pharmacol. Rev., 67:505 (2015).

Reinhardt et al., Tumour Biol., 39 (2017).

Robertson, Oncologist, 12:774 (2007).

Rodriguez-Gonzalez et al., Oncogene, 27:7201 (2008).

Rothenberger et al., Int. J. Mol. Sci., 19: (2018).

Rouhimoghadam et al., Frontiers in Endocrinology, 11:877 (2020).

Rouhimoghadam et al., Front Physiol., 9:907 (2018).

Rugo et al., J. Clin. Oncol., 34:3069 (2016).

Sakamoto et al., Mol. Cell Proteomics, 2:1350 (2003).

Sammons et al., Target Oncol., 14:1 (2019).

Schneekloth et al., Bioorg. Med. Chem. Lett., 18:5904 (2008).

Silva et al., Elife 8: (2019).

Sjöström et al., Breast Cancer Res. Treat., 145:61 (2014).

Smith et al., Am. J. Obstet. Gynecol., 196:386 (2007).

Smith et al., Gynecol. Oncol., 114:465 (2009).

Steiman et al., Am. J. Surg., 206:698 (2013).

Steinebach et al., Chem. Commun. (Camb), 55:1821 (2019).

Stevis et al., Endocrinology, 140:5455 (1999).

Sun et al., Signal Transduct. Target Ther., 4:64 (2019).

Tanenbaum et al., Proc. Natl. Acad. Sci. USA, 95:5998 (1998).

Thangavel et al., Endocr. Relat. Cancer, 18:333 (2011).

Thomas et al., Endocrinology, 146:624 (2005).

Tremont et al., OchsnerJ., 17:405 (2017).

Tria et al., J. Med. Chem., 61:2837 (2018).

Vladusic et al., Cancer Res., 58:210 (1998).

Waters et al., J. Neurosci., 35:2384 (2015). PMID: 25673833.

Weißenborn et al., J. Cancer Res. Clin. Oncol., 140:713 (2014).

Winter et al., Science, 348:1376 (2015).

Yang et al., Pharmacol. Ther., 139:392 (2013).

Yin et al., Int. J. Oncol., 51:1191 (2017).

Yu et al., J. Steroid Biochem. Mol. Biol., 143:392 (2014).

Yue et al., Int. J. Cancer, 127:1748 (2010).

Zhang et al., Bioorg. Med. Chem. Lett., 14:645 (2004).

Zhao et al., Neoplasia, 22:522 (2020).

Zhou et al. J. Clin. Invest., 125:2123 (2015).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A molecule comprising a G-protein coupled estrogen receptor (GPER) ligand coupled to a linker coupled to an E3 ubiquitin ligase ligand.

2. The molecule of claim 1 wherein the GPER ligand comprises 17β-estradiol, estrone, a phytoestrogen, a xenoestrogen, estriol, estriol 3-sulfate, estriol 17-sulfate, G-1, G-15, G-36, genistein, dazine, or quercetin.

3. The molecule of claim 2 wherein the phytoestrogen comprises a flavone, isoflavone, lignin saponin, coumestin, or stilbene.

4. The molecule of claim 1 wherein the GPER ligand is a GPER antagonist.

5. The molecule of claim 1 wherein the E3 ubiquitin ligase ligand is a Von Hippel ligase (VHL) ligand.

6. The molecule of claim 1 wherein the E3 ubiquitin ligase ligand comprises cereblon, lenalidomide, pomalidomide, iberdomide, (S,R,S)-AHPC, thalidomide, VH-298, CC-122, CC-885, E3ligase ligand 8, TD-106, VL285, VH032, VH101, VH298, VHL ligand 4, VHL ligand 7, VHL-2 ligand 3, E3 ligase ligand 3, E3 ligase ligand 2, or BC-1215.

7. The molecule of claim 1 wherein the linker has a chain having 10 to 50 atoms.

8. The molecule of claim 7 wherein the linker is an alkyl linker or a heteroalkyl linker or comprises polyethylene glycol (PEG).

9-10. (canceled)

11. The molecule of claim 8 wherein the linker comprises 3 to 15 PEG units or comprises (PEG)ₘNH(CO)(PEG)ₙ where n and m independently are 0, 1, 2, 3, 4, 5, 6, 7, 8, 9, 10, 11, 12, 13, 14 or 15.

12. (canceled)

13. The molecule of claim 11 wherein n is 3, 4, 5, or 6 or wherein m is 7, 8, 9, or 10.

14. (canceled)

15. A pharmaceutical composition comprising an amount of the molecule of claim 1.

16-19. (canceled)

20. A method to prevent, inhibit or treat a cancer in a mammal, comprising: administering to the mammal a composition having an effective amount of a molecule comprising a G-protein coupled estrogen receptor (GPER) ligand coupled to a linker coupled to an E3 ubiquitin ligase ligand.

21. The method of claim 20 wherein the cancer is a GPER positive cancer.

22. The method of claim 20 wherein the cancer is an endocrine resistant cancer or a hormonal therapy resistant cancer.

23. (canceled)

24. The method of claim 20 wherein the cancer is a triple negative breast cancer.

25. The method of claim 20 wherein the cancer is a gynecological cancer.

26. The method of claim 20 wherein the cancer is ovarian cancer or endometrial cancer.

27. (canceled)

28. The method of claim 20 wherein the mammal is a human.

29. The method of claim 20 8 wherein the administration is systemic.

30. (canceled)

31. The method of claim 20 wherein the composition is a sustained release dosage form.

32. (canceled)


SEQUENCE LISTING (5 sequences)

SEQ ID NO: 1 - DNA, 34 bp, Artificial Sequence
  A synthetic sequence
gttcaagaagattagcgatgtgacttcccaagcc

SEQ ID NO: 2 - DNA, 35 bp, Artificial Sequence
  A synthetic sequence
agccgccagccgctcaccatgtctctgcaccgtgc

SEQ ID NO: 3 - DNA, 42 bp, Artificial Sequence
  A synthetic sequence
tggcggctgttcaagaagattagctgagagctccctggcgga

SEQ ID NO: 4 - DNA, 43 bp, Artificial Sequence
  A synthetic sequence
gccgctcacagagcctcctccaccgactgtggcagggaaaccc

SEQ ID NO: 5 - protein, 11 aa, Artificial Sequence
  A synthetic sequence
VSGWRLFKKIS